(19) United States
(12) Patent Application Publication
(10) Pub. No.: US 2025/0034154 A1
(43) Pub. Date: Jan. 30, 2025

(54) SALTS AND SOLID FORMS OF A COMPOUND THAT MODULATES IRAK4

(71) Applicant: Gilead Sciences, Inc., Foster City, CA (US)

(72) Inventors: Stephen E. AMMANN, Foster City, CA (US); Peter C. FUNG, San Mateo, CA (US); Brittanie T. HOANG, New York, NY (US); Elizabeth M. HORSTMAN, San Carlos, CA (US); Stephen LAU, South San Francisco, CA (US); Henry G. MORRISON, Dublin, CA (US); Devon A. MUNDAL, Seattle, WA (US); May G. YOUNG, Fremont, CA (US); Chia-Yun YU, San Mateo, CA (US)

(21) Appl. No.: 18/759,283

(22) Filed: Jun. 28, 2024

Related U.S. Application Data
(60) Provisional application No. 63/511,597, filed on Jun. 30, 2023.

(51) Int. Cl. C07D 487/04; A61K 31/5025

(52) U.S. Cl. CPC C07D 487/04; A61K 31/5025; C07B 2200/13

(57) ABSTRACT

The present disclosure relates to salts and solid forms of a compound that are inhibitors of the kinase IRAK4 and their uses as therapeutic agents for treating diseases, disorders, or conditions modulated by IRAK4, such as inflammatory bowel disease (IBD), systemic lupus erythematosus (SLE), psoriasis, or rheumatoid arthritis.

### CROSS-REFERENCE TO RELATED APPLICATIONS

This application claims the benefit under 35 U.S.C. § 119(e) of U.S. Provisional Application Ser. No. 63/511,597, filed Jun. 30, 2023, the contents of which are hereby incorporated by reference in its entirety.

### FIELD

The present disclosure relates generally to salts and solid forms of a compound that are inhibitors of the kinase IRAK4. The disclosure also relates to pharmaceutical compositions comprising such salts and solid forms and methods of using the same.

### BACKGROUND

Interleukin-1 receptor-associated kinase-4 (IRAK4) is a serine-threonine kinase that acts as a mediator in interleukin-1/Toll-like receptor (IL-1/TLR) signaling cascades. More particularly, IRAK4 is involved in activation of adaptor protein myeloid differentiation primary response gene 88 (MyD88) signaling cascades and is hypothesized to play a role in inflammatory and fibrotic disorders, such as rheumatoid arthritis (RA), inflammatory bowel disease (IBD), gout, Lyme disease, arthritis, psoriasis, pelvic inflammatory disease, systemic lupus erythematosus (SLE), Sjogren's syndrome, viral myocarditis, acute and chronic tissue injury, non-alcoholic steatohepatitis (NASH), alcoholic hepatitis, and kidney disease, including chronic kidney disease and diabetic kidney disease. In addition, IRAK4 plays a role in certain cancers and is hypothesized to play a role in inflammation associated with gastrointestinal infections, including C. difficile. Signaling through IL-1R/TLR results in the activation of MyD88 which recruits IRAK4 and IRAK1 to form a signaling complex. This complex then interacts with a series of kinases, adaptor proteins, and ligases, ultimately resulting in the activation of nuclear factor kappa-light-chain-enhancer of activated B cells (NF-κB), activator protein-1 (API), cyclic AMP-responsive element-binding protein (CREB) and the interferon-regulatory factors (IRFs), including IRF5 and IRF7, inducing the generation of pro-inflammatory cytokines and type I interferons.

Therefore, inhibitors of IRAK4 may be useful in the treatment of inflammatory and fibrotic disorders, such as rheumatoid arthritis (RA), inflammatory bowel disease (IBD), gout, Lyme disease, arthritis, psoriasis, pelvic inflammatory disease, systemic lupus erythematosus (SLE), Sjogren's syndrome, inflammation associated with gastrointestinal infections, including C. difficile, viral myocarditis, acute and chronic tissue injury, non-alcoholic steatohepatitis (NASH), alcoholic hepatitis and kidney disease, including chronic kidney disease and diabetic kidney disease.

### SUMMARY

Provided herein are salts and solid forms of a compound that are useful as inhibitors of IRAK4. Also disclosed herein are pharmaceutical compositions comprising salts and solid forms of Compound I and methods of using the same in the treatment of diseases, disorders, or conditions modulated by IRAK4.

Some embodiments provide for a crystalline form of Compound I:

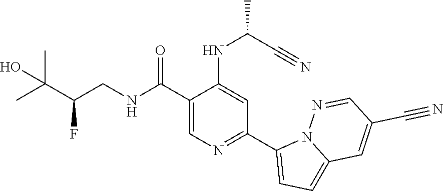

(Compound I Form I), wherein Compound I Form I is characterized by an X-ray powder diffractogram comprising peaks (±0.2°) at 7.5, 12.3, and 7.2°2θ as determined on a diffractometer using Cu-Kα radiation.

Some embodiments provide for a crystalline form of Compound I (Compound I Form II), wherein Compound I Form II is characterized by an X-ray powder diffractogram comprising peaks (±0.2°) at 7.5, 14.6, and 17.8°2θ as determined on a diffractometer using Cu-Kα radiation.

Some embodiments provide for a crystalline form of Compound I (Compound I Form IV), wherein Compound I Form IV is characterized by an X-ray powder diffractogram comprising peaks (±0.2°) at 10.1, 10.7, and 17.9°2θ as determined on a diffractometer using Cu-Kα radiation.

Some embodiments provide for a crystalline form of Compound I (Compound I Form V), wherein Compound I Form V is a monohydrate and characterized by an X-ray powder diffractogram comprising peaks (±0.2°) at 11.8, 25.9, and 20.7°2θ as determined on a diffractometer using Cu-Kα radiation.

Some embodiments provide for a crystalline form of Compound I (Compound I Form VI), wherein Compound I Form VI is a monohydrate and characterized by an X-ray powder diffractogram comprising peaks (±0.2°) at 4.9, 5.6, and 7.4°2θ as determined on a diffractometer using Cu-Kα radiation.

Some embodiments provide for a crystalline form of a mono-citrate salt of Compound I (Compound I mono-citrate Form I), wherein Compound I mono-citrate Form I is characterized by an X-ray powder diffractogram comprising peaks (±0.2°) at 5.7, 7.0, and 22.7°2θ as determined on a diffractometer using Cu-Kα radiation.

Some embodiments provide for a crystalline form of Compound I (Compound I Form III), wherein Compound I Form III is characterized by an X-ray powder diffractogram comprising peaks (±0.2°) at 21.2, 12.3, and 10.7°2θ as determined on a diffractometer using Cu-Kα radiation; or
(Compound I Form VII), wherein Compound I Form VII is a water:tetrahydrofuran (THF)solvate and characterized by an X-ray powder diffractogram comprising peaks (±0.2°) at 4.0, 25.8, and 6.9°2θ as determined on a diffractometer using Cu-Kα radiation; or
(Compound I Form VIII), wherein Compound I Form VIII is a water:acetonitrile (ACN) solvate and characterized by an X-ray powder diffractogram comprising peaks (±0.2°) at 8.0, 25.8, and 6.1°2θ as determined on a diffractometer using Cu-Kα radiation; or
(Compound I Form IX), wherein Compound I Form IX is a water:2-methyl tetrahydrofuran (MeTHF) solvate and characterized by an X-ray powder diffractogram comprising peaks (±0.2°) at 9.4, 4.5, and 18.3°2θ as determined on a diffractometer using Cu-Kα radiation; or
(Compound I Form X), wherein Compound I Form X is an isopropyl alcohol (IPA) solvate and characterized by an X-ray powder diffractogram comprising peaks (±0.2°) at 4.0, 7.0, and 8.2°2θ as determined on a diffractometer using Cu-Kα radiation; or
(Compound I Form XI), wherein Compound I Form XI is a methyl isobutyl ketone (MIBK) solvate and characterized by an X-ray powder diffractogram comprising peaks (±0.2°) at 4.0, 6.9, and 14.5°2θ as determined on a diffractometer using Cu-Kα radiation; or
(Compound I Form XII), wherein Compound I Form XII is characterized by an X-ray powder diffractogram comprising peaks (±0.2°) at 7.7, 11.7 and 26.1°2θ as determined on a diffractometer using Cu-Kα radiation; or
(Compound I Form XIII), wherein Compound I Form XIII is characterized by an X-ray powder diffractogram comprising peaks (±0.2°) at 7.6, 14.6, and 17.9°2θ as determined on a diffractometer using Cu-Kα radiation; or
(Compound I mono-citrate Form II), wherein Compound I mono-citrate Form II is a mono-citrate salt and characterized by an X-ray powder diffractogram comprising peaks (±0.2°) at 5.6, 7.0, and 24.2°2θ as determined on a diffractometer using Cu-Kα radiation; or
(Compound I mono-citrate Form III), wherein Compound I mono-citrate Form III is a mono-citrate salt and characterized by an X-ray powder diffractogram comprising peaks (±0.2°) at 6.3, 18.8, and 7.7°2θ as determined on a diffractometer using Cu-Kα radiation; or
(Compound I mono-citrate Form IV), wherein Compound I mono-citrate Form IV is a mono-citrate salt characterized by an X-ray powder diffractogram comprising peaks (±0.2°) at 26.8, 25.7, and 25.1°2θ as determined on a diffractometer using Cu-Kα radiation; or
(Compound I hemi-citrate Form I), wherein Compound I hemi-citrate Form I is a hemi-citrate salt characterized by an X-ray powder diffractogram comprising peaks (±0.2°) at 6.1, 7.4, and 17.1°2θ as determined on a diffractometer using Cu-Kα radiation; or
(Compound I mono-HCl Form I), wherein Compound I mono-HCl Form I is a mono-hydrochloride (HCl) salt and characterized by an X-ray powder diffractogram comprising peaks (±0.2°) at 6.9, 20.7, and 16.9°2θ as determined on a diffractometer using Cu-Kα radiation; or
(Compound I mono-maleate Form I), wherein Compound I mono-maleate Form I is a mono-maleate salt and characterized by an X-ray powder diffractogram comprising peaks (±0.2°) at 7.4, 6.1, and 23.2°2θ as determined on a diffractometer using Cu-Kα radiation; or
(Compound I hemi-fumarate Form I), wherein Compound I hemi-fumarate Form I is a hemi-fumarate salt and characterized by an X-ray powder diffractogram comprising peaks (±0.2°) at 7.7, 6.8, and 13.0°2θ as determined on a diffractometer using Cu-Kα radiation; or
(Compound I mono-fumarate Form I), wherein Compound I mono-fumarate Form I is a mono-fumarate salt and characterized by an X-ray powder diffractogram comprising peaks (±0.2°) at 6.1, 7.8, and 18.7°2θ as determined on a diffractometer using Cu-Kα radiation; or
(Compound I hemi-L-tartrate Form I), wherein Compound I hemi-L-tartrate Form I is a hemi-L-tartrate salt and characterized by an X-ray powder diffractogram comprising peaks (±0.2°) at 8.5, 5.2 and 18.6°2θ as determined on a diffractometer using Cu-Kα radiation; or
(Compound I mono-ESA Form I), wherein Compound I mono-ethanesulfonic acid (ESA) Form I is a mono-ESA salt and characterized by an X-ray powder diffractogram comprising peaks (±0.2°) at 6.4, 10.9, and 19.0°2θ as determined on a diffractometer using Cu-Kα radiation; or
(Compound I hemi-glycolate Form I), wherein Compound I hemi-glycolate Form I is a hemi-glycolate and characterized by an X-ray powder diffractogram comprising peaks (±0.2°) at 6.2, 8.0, and 22.9°2θ as determined on a diffractometer using Cu-Kα radiation; or
(Compound I sulfate Form I), wherein Compound I sulfate Form I is a sulfate salt and characterized by an X-ray powder diffractogram comprising peaks (±0.2°) at 5.5, 4.9, and 11.1°2θ as determined on a diffractometer using Cu-Kα radiation; or
(Compound I phosphate Form I), wherein Compound I phosphate Form I is a phosphate salt characterized by an X-ray powder diffractogram comprising peaks (±0.2°) at 4.8, 9.7, and 16.9°2θ as determined on a diffractometer using Cu-Kα radiation; or
(Compound I hemi-HCl Form I), wherein Compound I hemi-HCl Form I is a hemi-hydrochloride (HCl) salt characterized by an X-ray powder diffractogram comprising peaks (±0.2°) at 5.6, 9.2, and 11.2°2θ as determined on a diffractometer using Cu-Kα radiation; or
(Compound I co-crystal Form I), wherein Compound I co-crystal Form I is a co-crystal of Compound I and 4-(((5)-1-cyanoethyl)amino)-6-(3-cyanopyrrolo[1,2-b]pyridazin-7-yl)-N—((R)-2-fluoro-3-hydroxy-3-methylbutyl)nicotinamide and is characterized by an X-ray powder diffractogram comprising peaks (±0.2°) at 19.1, 10.3, and 9.4°2θ as determined on a diffractometer using Cu-Kα radiation.

Some embodiments provide for a pharmaceutical composition comprising a crystalline form as described herein and a pharmaceutically acceptable carrier.

### BRIEF DESCRIPTION OF THE DRAWINGS

FIG. 1 shows an X-ray powder diffraction (XRPD) pattern of Compound I Form I.

FIG. 2 shows a differential scanning calorimetry (DSC) curve of Compound I Form I.

FIG. 3 shows thermogravimetric analysis (TGA) of Compound I Form I.

FIG. 4 shows an X-ray powder diffraction (XRPD) pattern of Compound I Form II.

FIG. 5 shows a differential scanning calorimetry (DSC) curve of Compound I Form II.

FIG. 6 shows thermogravimetric analysis (TGA) of Compound I Form II.

FIG. 7 shows an X-ray powder diffraction (XRPD) pattern of Compound I Form III.

FIG. 8 shows an X-ray powder diffraction (XRPD) pattern of Compound I Form IV.

FIG. 9 shows a differential scanning calorimetry (DSC) curve of Compound I Form IV.

FIG. 10 shows thermogravimetric analysis (TGA) of Compound I Form IV.

FIG. 11 shows a dynamic vapor sorption (DVS) curve of Compound I Form IV.

FIG. 12 shows an X-ray powder diffraction (XRPD) pattern of Compound I Form V.

FIG. 13 shows a differential scanning calorimetry (DSC) curve of Compound I Form V.

FIG. 14 shows thermogravimetric analysis (TGA) of Compound I Form V.

FIG. 15 shows a dynamic vapor sorption (DVS) curve of Compound I Form V.

FIG. 16 shows an X-ray powder diffraction (XRPD) pattern of Compound I Form VI.

FIG. 17 shows a differential scanning calorimetry (DSC) curve of Compound I Form VI.

FIG. 18 shows thermogravimetric analysis (TGA) of Compound I Form VI.

FIG. 19 shows a dynamic vapor sorption (DVS) curve of Compound I Form VI.

FIG. 20 shows an X-ray powder diffraction (XRPD) pattern of Compound I Form VII.

FIG. 21 shows a differential scanning calorimetry (DSC) curve of Compound I Form VII.

FIG. 22 shows thermogravimetric analysis (TGA) of Compound I Form VII.

FIG. 23 shows a dynamic vapor sorption (DVS) curve of Compound I Form VII.

FIG. 24 shows an X-ray powder diffraction (XRPD) pattern of Compound I Form VIII.

FIG. 25 shows a differential scanning calorimetry (DSC) curve of Compound I Form VIII.

FIG. 26 shows thermogravimetric analysis (TGA) of Compound I Form VIII.

FIG. 27 shows a dynamic vapor sorption (DVS) curve of Compound I Form VIII.

FIG. 28 shows an X-ray powder diffraction (XRPD) pattern of Compound I Form IX.

FIG. 29 shows a differential scanning calorimetry (DSC) curve of Compound I Form IX.

FIG. 30 shows thermogravimetric analysis (TGA) of Compound I Form IX.

FIG. 31 shows a dynamic vapor sorption (DVS) curve of Compound I Form IX.

FIG. 32 shows an X-ray powder diffraction (XRPD) pattern of Compound I Form X.

FIG. 33 shows a differential scanning calorimetry (DSC) curve of Compound I Form X.

FIG. 34 shows thermogravimetric analysis (TGA) of Compound I Form X.

FIG. 35 shows an X-ray powder diffraction (XRPD) pattern of Compound I Form XI.

FIG. 36 shows an X-ray powder diffraction (XRPD) pattern of Compound I Form XII.

FIG. 37 shows an X-ray powder diffraction (XRPD) pattern of Compound I Form XIII.

FIG. 38 shows an X-ray powder diffraction (XRPD) of Compound I amorphous Form.

FIG. 39 shows an X-ray powder diffraction (XRPD) pattern of Compound I mono-citrate Form I.

FIG. 40 shows a differential scanning calorimetry (DSC) curve of Compound I mono-citrate Form I.

FIG. 41 shows thermogravimetric analysis (TGA) of Compound I mono-citrate Form I.

FIG. 42 shows a dynamic vapor sorption (DVS) curve of Compound I mono-citrate Form I.

FIG. 43 shows a proton nuclear magnetic resonance (¹H NMR) of Compound I mono-citrate Form I.

FIG. 44 shows an X-ray powder diffraction (XRPD) pattern of Compound I mono-citrate Form II.

FIG. 45 shows an X-ray powder diffraction (XRPD) pattern of Compound I mono-citrate Form III.

FIG. 46 shows a differential scanning calorimetry (DSC) curve of Compound I mono-citrate Form III.

FIG. 47 shows thermogravimetric analysis (TGA) of Compound I mono-citrate Form III.

FIG. 48 shows a dynamic vapor sorption (DVS) curve of Compound I mono-citrate Form III.

FIG. 49 shows an X-ray powder diffraction (XRPD) pattern of Compound I mono-citrate Form IV.

FIG. 50 shows a differential scanning calorimetry (DSC) curve of Compound I mono-citrate Form IV.

FIG. 51 shows thermogravimetric analysis (TGA) of Compound I mono-citrate Form IV.

FIG. 52 shows a dynamic vapor sorption (DVS) curve of Compound I mono-citrate Form IV.

FIG. 53 shows an X-ray powder diffraction (XRPD) pattern of Compound I hemi-citrate Form I.

FIG. 54 shows a differential scanning calorimetry (DSC) curve of Compound I hemi-citrate Form I.

FIG. 55 shows thermogravimetric analysis (TGA) of Compound I hemi-citrate Form I.

FIG. 56 shows a dynamic vapor sorption (DVS) curve of Compound I hemi-citrate Form I.

FIG. 57 shows a proton nuclear magnetic resonance (¹H NMR) of Compound I hemi-citrate Form I.

FIG. 58 shows an X-ray powder diffraction (XRPD) pattern of Compound I mono-HCl Form I.

FIG. 59 shows a differential scanning calorimetry (DSC) curve of Compound I mono-HCl Form I.

FIG. 60 shows thermogravimetric analysis (TGA) of Compound I mono-HCl Form I.

FIG. 61 shows a dynamic vapor sorption (DVS) curve of Compound I mono-HCl Form I.

FIG. 62 shows an X-ray powder diffraction (XRPD) pattern of Compound I mono-maleate Form I.

FIG. 63 shows a differential scanning calorimetry (DSC) curve of Compound I mono-maleate Form I.

FIG. 64 shows thermogravimetric analysis (TGA) of Compound I mono-maleate Form I.

FIG. 65 shows a dynamic vapor sorption (DVS) curve of Compound I mono-maleate Form I.

FIG. 66 shows a proton nuclear magnetic resonance (¹H NMR) of Compound I mono-maleate Form I.

FIG. 67 shows an X-ray powder diffraction (XRPD) pattern of Compound I hemi-fumarate Form I.

FIG. 68 shows a differential scanning calorimetry (DSC) curve of Compound I hemi-fumarate Form I.

FIG. 69 shows thermogravimetric analysis (TGA) of Compound I hemi-fumarate Form I.

FIG. 70 shows a dynamic vapor sorption (DVS) curve of Compound I hemi-fumarate Form I.

FIG. 71 shows a proton nuclear magnetic resonance (¹H NMR) of Compound I hemi-fumarate Form I.

FIG. 72 shows an X-ray powder diffraction (XRPD) pattern of Compound I mono-fumarate Form I.

FIG. 73 shows a differential scanning calorimetry (DSC) curve of Compound I mono-fumarate Form I.

FIG. 74 shows thermogravimetric analysis (TGA) of Compound I mono-fumarate Form I.

FIG. 75 shows a dynamic vapor sorption (DVS) curve of Compound I mono-fumarate Form I.

FIG. 76 shows a proton nuclear magnetic resonance (¹H NMR) of Compound I mono-fumarate Form I.

FIG. 77 shows an X-ray powder diffraction (XRPD) pattern of Compound I hemi-L-tartrate Form I.

FIG. 78 shows a differential scanning calorimetry (DSC) curve of Compound I hemi-L-tartrate Form I.

FIG. 79 shows thermogravimetric analysis (TGA) of Compound I hemi-L-tartrate Form I.

FIG. 80 shows a dynamic vapor sorption (DVS) curve of Compound I hemi-L-tartrate Form I.

FIG. 81 shows a proton nuclear magnetic resonance (¹H NMR) of Compound I hemi-L-tartrate Form I.

FIG. 82 shows an X-ray powder diffraction (XRPD) pattern of Compound I mono-ESA Form I.

FIG. 83 shows a differential scanning calorimetry (DSC) curve of Compound I mono-ESA Form I.

FIG. 84 shows thermogravimetric analysis (TGA) of Compound I mono-ESA Form I.

FIG. 85 shows a dynamic vapor sorption (DVS) curve of Compound I mono-ESA Form I.

FIG. 86 shows a proton nuclear magnetic resonance (¹H NMR) of Compound I mono-ESA Form I.

FIG. 87 shows an X-ray powder diffraction (XRPD) pattern of Compound I hemi-glycolate Form I.

FIG. 88 shows a differential scanning calorimetry (DSC) curve of Compound I hemi-glycolate Form I.

FIG. 89 shows thermogravimetric analysis (TGA) of Compound I hemi-glycolate Form I.

FIG. 90 shows a dynamic vapor sorption (DVS) curve of Compound I hemi-glycolate Form I.

FIG. 91 shows a proton nuclear magnetic resonance (¹H NMR) of Compound I hemi-glycolate Form I.

FIG. 92 shows an X-ray powder diffraction (XRPD) pattern of Compound I sulfate Form I.

FIG. 93 shows a differential scanning calorimetry (DSC) curve of Compound I sulfate Form I.

FIG. 94 shows thermogravimetric analysis (TGA) of Compound I sulfate Form I.

FIG. 95 shows an X-ray powder diffraction (XRPD) pattern of Compound I phosphate Form I.

FIG. 96 shows a differential scanning calorimetry (DSC) curve of Compound I phosphate Form I.

FIG. 97 shows thermogravimetric analysis (TGA) of Compound I phosphate Form I.

FIG. 98 shows an X-ray powder diffraction (XRPD) pattern of Compound I hemi-HCl Form I.

FIG. 99 shows a differential scanning calorimetry (DSC) curve of Compound I hemi-HCl Form I.

FIG. 100 shows thermogravimetric analysis (TGA) of Compound I hemi-HCl Form I.

FIG. 101 shows a dynamic vapor sorption (DVS) curve of Compound I hemi-HCl Form I.

FIG. 102 shows an X-ray powder diffraction (XRPD) pattern of Compound I co-crystal Form I.

FIG. 103 shows a differential scanning calorimetry (DSC) curve of Compound I co-crystal Form I.

FIG. 104 shows thermogravimetric analysis (TGA) of Compound I co-crystal Form I.

### DETAILED DESCRIPTION

The compound, 4-(((R)-1-cyanoethyl)amino)-6-(3-cyanopyrrolo[1,2-b]pyridazin-7-yl)-N—((R)-2-fluoro-3-hydroxy-3-methylbutyl)nicotinamide, designated herein as Compound I, has the following formula:

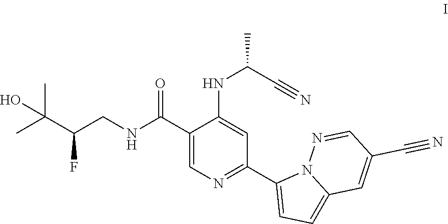

Compound I is an inhibitor of IRAK4. The synthesis and method of use thereof is described in PCT International Application Publication No. WO 2020/014468, U.S. Pat. No. 10,875,866, U.S. Application No. 63/511,558 (filed on Jun. 30, 2023, and titled “Processes for Making IRAK4 Inhibitors”), and PCT Application No. PCT/US2024/036103 (which claims priority to U.S. Application No. 63/511,558, and which is filed on even date herewith, and titled “Processes for Making IRAK4 Inhibitors”), each of which are herein incorporated by reference in its entirety. Unless otherwise specified, reference to Compound I is intended to encompass the compound per se, or a salt, such as a pharmaceutically acceptable salt, stereoisomer, mixture of stereoisomers, solid form, co-crystal, solvate, and/or hydrate thereof.

### Definitions

As used in the present specification, the following words and phrases are generally intended to have the meanings as set forth below, except to the extent that the context in which they are used indicates otherwise.

The term “comprise” and variations thereof, such as, “comprises” and “comprising” are to be construed in an open, inclusive sense, that is, as “including, but not limited to.” Further, the singular forms “a,” “an,” and “the” include plural references unless the context clearly dictates otherwise. Thus, reference to “the compound” includes a plurality of such compounds, and reference to “the assay” includes reference to one or more assays and equivalents thereof known to those skilled in the art.

Reference to “about” a value or parameter herein includes (and describes) embodiments that are directed to that value or parameter per se. In certain embodiments, the term “about” includes the indicated amount ±10%. In other embodiments, the term “about” includes the indicated amount ±5%. In certain other embodiments, the term “about” includes the indicated amount ±2.5%. In certain other embodiments, the term “about” includes the indicated amount ±1%. Also, to the term “about X” includes description of “X”.

Recitation of numeric ranges of values throughout the disclosure is intended to serve as a shorthand notation of referring individually to each separate value falling within the range inclusive of the values defining the range, and each separate value is incorporated in the specification as it were individually recited herein.

Forms of Compound I or salts, co-crystals, solvates, or hydrates thereof are provided herein. In one embodiment, reference to a form of Compound I or a salt, co-crystal, solvate, or hydrate thereof means that at least 50% to 99% (e.g., at least 50%, at least 55%, at least 60%, at least 65%, at least 70%, at least 75%, at least 80%, at least 85%, at least 90%, at least 95%, or at least 99%) of Compound I or a salt, co-crystal, solvate, or hydrate thereof is present in a composition in the designated form. For instance, in one embodiment, reference to of Compound I Form I means that at least 50%, at least 55%, at least 60%, at least 65%, at least 70%, at least 75%, at least 80%, at least 85%, at least 90%, at least 95%, or at least 99% of Compound I is present in a composition as Form I.

The term “solid form” refers to a type of solid-state material that includes amorphous as well as crystalline forms. The term “crystalline form” refers to polymorphs as well as solvates, hydrates, etc. The term “polymorph” refers to a particular crystal structure having particular physical properties such as X-ray diffraction, melting point, and the like.

As used herein, the term “salt” refers to a compound formed by the reaction of an acid and a base, resulting in the formation of a positively charged cation and a negatively charged anion. In general, a salt is defined as a compound that is formed by the combination of positively and negatively charged ions, where the charges of the ions result in a neutral compound. Salts can be either inorganic or organic. As used herein, the term “salt” includes partially or fully ionized salt forms. In some embodiments, the salt is fully ionized.

The term “co-crystal” refers to a molecular complex of a compound disclosed herein and one or more non-ionized co-crystal formers connected via non-covalent interactions. In some embodiments, the co-crystals disclosed herein may include a non-ionized form of Compound I (e.g., Compound I free form) and one or more non-ionized co-crystal formers, where non-ionized Compound I and the co-crystal former(s) are connected through non-covalent interactions. In some embodiments, co-crystals disclosed herein may include an ionized form of Compound I (e.g., a salt of Compound I) and one or more non-ionized co-crystals formers, where ionized Compound I and the co-crystal former(s) are connected through non-covalent interactions. Co-crystals may additionally be present in anhydrous, solvated or hydrated forms. In certain instances, co-crystals may have improved properties as compared to the parent form (i.e., the free molecule, zwitterion, etc.) or a salt of the parent compound. Improved properties can be increased solubility, increased dissolution, increased bioavailability, increased dose response, decreased hygroscopicity, increased stability, a crystalline form of a normally amorphous compound, a crystalline form of a difficult to salt or unsaltable compound, decreased form diversity, more desired morphology, and the like. Methods for making and characterizing co-crystals are known to those of skill in the art.

The term “co-crystal former” or “co-former” refers to one or more pharmaceutically acceptable bases or pharmaceutically acceptable acids disclosed herein in association with Compound I, or any other compound disclosed herein. Such bases include ethanolamine, diethanolamine, triethanolamine, tromethamine, N-methylglucamine, and the like. Such acids include inorganic acids such as hydrochloric acid, sulfuric acid, nitric acid, phosphoric acid, and the like; or organic acids such as formic acid, acetic acid, propionic acid, glycolic acid, pyruvic acid, lactic acid, malonic acid, maleic acid, tartaric acid, citric acid, methanesulfonic acid, ethanesulfonic acid, gluconic acid, glutamic acid, salicylic acid, stearic acid, and the like.

The term “solvate” refers to a complex formed by combination of solvent molecules with molecules or ions of the solute. The solvent can be an organic compound, an inorganic compound, or a mixture of both. As used herein, the term “solvate” includes a “hydrate” (i.e., a complex formed by combination of water molecules with molecules or ions of the solute), hemi-hydrate, channel hydrate, etc. Some examples of solvents include, but are not limited to, acetonitrile, methanol, N,N-dimethylformamide, tetrahydrofuran, 2-methyltetrahydrofuran, dimethylsulfoxide, and water. In general, the solvated forms are equivalent to unsolvated forms and are encompassed within the scope of the present disclosure.

The term “desolvated” refers to a Compound I form that is a solvate as described herein, and from which solvent molecules have been partially or completely removed. Desolvation techniques to produce desolvated forms include, without limitation, exposure of a Compound I form (solvate) to a vacuum, subjecting the solvate to elevated temperature, exposing the solvate to a stream of gas, such as air or nitrogen, or any combination thereof. Thus, a desolvated or “unsolvated” Compound I form can be anhydrous, i.e., completely without solvent molecules, or partially solvated wherein solvent molecules are present in stoichiometric or non-stoichiometric amounts.

The term “amorphous” refers to a state in which the material lacks long range order at the molecular level and, depending upon temperature, may exhibit the physical properties of a solid or a liquid. Typically such materials do not give distinctive X-ray diffraction patterns and, while exhibiting the properties of a solid, are more formally described as a liquid. Upon heating, a change from solid to liquid properties occurs which is characterized by a change of state, typically second order (glass transition).

“Stereoisomers” are isomers that differ only in the way the atoms are arranged in space.

Any formula or structure given herein, including Compound I, is also intended to represent unlabeled forms as well as isotopically labeled forms of the compounds. It is understood that for any given atom, the isotopes may be present essentially in ratios according to their natural occurrence, or one or more particular atoms may be enhanced with respect to one or more isotopes using synthetic methods known to one skilled in the art. Thus, hydrogen includes for example ¹H, ²H, ³H; carbon includes for example ¹¹C, ¹²C ¹³C, ¹⁴C; oxygen includes for example ¹⁶O, ¹⁷O, ¹⁸O; nitrogen includes for example ¹³N, ¹⁴N, ¹⁵N; sulfur includes for example ³²S, ³³S, ³⁴S, ³⁵S, ³⁶S, ³⁷S, ³⁸S; fluoro includes for example ¹⁷F, ¹⁸F, ¹⁹F; chloro includes for example ³⁵Cl, ³⁶Cl, ³⁷Cl, ³⁸Cl, ³⁹Cl; and the like.

As used herein, the terms “treat,” “treating,” “therapy,” “therapies,” and like terms refer to the administration of material, e.g., any one or more solid, crystalline or polymorphs of Compound I as described herein in an amount effective to prevent, alleviate, or ameliorate one or more symptoms of a disease or condition, i.e., indication, and/or to prolong the survival of the subject being treated.

The term “administering” refers to oral administration, administration as a suppository, topical contact, intravenous, intraperitoneal, intramuscular, intralesional, intranasal or subcutaneous administration, or the implantation of a slow-release device e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc.

As used herein, the term “modulating” or “modulate” refers to an effect of altering a biological activity, especially a biological activity associated with a particular biomolecule such as IRAK4 activity. For example, an agonist or antagonist of a particular biomolecule modulates the activity of the IRAK4 by either increasing (e.g. agonist, activator), or decreasing (e.g. antagonist, inhibitor) the activity, of the biomolecule. Such activity is typically indicated in terms of an inhibitory concentration (IC₅₀) or excitation concentration (EC₅₀) of the compound for an inhibitor or activator, respectively. In some embodiments, the term “modulating” or “modulate” refers to inhibiting or inhibition of, for example, IRAK4.

As used herein, in some embodiments, the term “composition” refers to a pharmaceutical preparation suitable for administration to an intended subject for therapeutic purposes that contains at least one pharmaceutically active compound, including any solid form thereof. The composition may include at least one pharmaceutically acceptable component to provide an improved formulation of the compound, such as a suitable carrier or excipient.

As used herein, the term “subject” or “patient” refers to a living organism that is treated with compounds as described herein, including, but not limited to, any mammal, such as a human, other primates, sports animals, animals of commercial interest such as cattle, farm animals such as horses, or pets such as dogs and cats.

The term “pharmaceutically acceptable” indicates that the indicated material does not have properties that would cause a reasonably prudent medical practitioner to avoid administration of the material to a patient, taking into consideration the disease or conditions to be treated and the respective route of administration. For example, it is commonly required that such a material be essentially sterile, e.g., for injectables. The term “pharmaceutically acceptable salt” of a given compound refers to salts that retain the biological effectiveness and properties of the given compound, and which are not biologically or otherwise undesirable. Pharmaceutically acceptable base addition salts can be prepared from inorganic and organic bases. Salts derived from inorganic bases include, by way of example only, sodium, potassium, lithium, ammonium, calcium and magnesium salts. Salts derived from organic bases include, but are not limited to, salts of primary, secondary and tertiary amines. Specific examples of suitable amines include, by way of example only, isopropyl amine, trimethyl amine, diethyl amine, tri(iso-propyl) amine, tri(n-propyl) amine, ethanolamine, diethanolamine, 2-dimethylamino ethanol, lysine, arginine, histidine, caffeine, procaine, hydrabamine, choline, betaine, ethylenediamine, glucosamine, N-alkylglucamines, theobromine, purines, piperazine, piperidine, morpholine, N-ethylpiperidine, and the like. Pharmaceutically acceptable acid addition salts may be prepared from inorganic and organic acids. Salts derived from inorganic acids include hydrochloric acid, hydrobromic acid, sulfuric acid, nitric acid, phosphoric acid, and the like. Salts derived from organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like.

In the present context, the term “therapeutically effective” or “effective amount” indicates that the materials or amount of material is effective to prevent, alleviate, or ameliorate one or more symptoms of a disease or medical condition, and/or to prolong the survival of the subject being treated. The therapeutically effective amount will vary depending on the compound, the disorder or condition and its severity and the age, weight, etc., of the mammal to be treated. For example, an effective amount is an amount sufficient to effectuate a beneficial or desired clinical result. The effective amounts can be provided all at once in a single administration or in fractional amounts that provide the effective amount in several administrations. The precise determination of what would be considered an effective amount may be based on factors individual to each subject, including their size, age, injury, and/or disease or injury being treated, and amount of time since the injury occurred or the disease began. One skilled in the art will be able to determine the effective amount for a given subject based on these considerations which are routine in the art.

In some embodiments, the phrase “substantially shown in Figure” or “substantially as shown in Figure” as applied to an X-ray powder diffractogram is meant to include a variation of ±0.2°2θ or +0.1°2θ, as applied to DSC thermograms is meant to include a variation of ±3° Celsius, and as applied to thermogravimetric analysis (TGA) is meant to include a variation of ±2% in weight loss. In some embodiments, the phrase “substantially shown in Figure” or “substantially as shown in Figure” as applied to DVS curves is meant to include a variation of ±5%.

“Substantially pure form (of a polymorph),” in some embodiments, means that in the referenced material, at least 99.9% of the material is the referenced polymorph. “Substantially pure form (of a polymorph),” in some embodiments, means that in the referenced material, at least 99.5% of the material is the referenced polymorph. “Substantially pure form (of a polymorph),” in some embodiments, means that in the referenced material, at least 99% of the material is the referenced polymorph. “Substantially pure form (of a polymorph),” in some embodiments, means that in the referenced material, at least 98% of the material is the referenced polymorph. “Substantially pure form (of a polymorph),” in some embodiments, means that in the referenced material, at least 97% of the material is the referenced polymorph. “Substantially pure form (of a polymorph),” in some embodiments, means that in the referenced material, at least 96% of the material is the referenced polymorph. “Substantially pure form (of a polymorph),” in some embodiments, means that in the referenced material, at least 95% of the material is the referenced polymorph.

### Salts and Forms of Compound I

As described generally above, the present disclosure provides salts and solid forms, such as crystalline forms, of the compound, 4-(((R)-1-cyanoethyl)amino)-6-(3-cyanopyrrolo[1,2-b]pyridazin-7-yl)-N—((R)-2-fluoro-3-hydroxy-3-methylbutyl)nicotinamide (hereinafter “Compound I”), and salts, co-crystals, solvates, or hydrates thereof. Crystalline forms of Compound I and salts, co-crystals, solvates, or hydrates thereof, and other forms (e.g., amorphous forms) of Compound I and salts, co-crystals, solvates, or hydrates thereof are collectively referred to herein as “forms of Compound I.”

In some embodiments, Compound I is in free form, e.g., a free base. In some embodiments, Compound I is a salt. In some embodiments, Compound I is a pharmaceutically acceptable salt. In some embodiments, Compound I is a solvate. In some embodiments, Compound I is a hydrate. In some embodiments, Compound I is unsolvated. In some embodiments, Compound I is an anhydrate. In some embodiments, provided is a substantially pure form of a solid form of Compound I as described herein. In some embodiments, provided is a substantially pure form of a crystalline form of Compound I as described herein.

While not intending to be bound by any particular theory, certain solid forms are characterized by physical properties, e.g., stability, solubility, and dissolution rate, appropriate for pharmaceutical and therapeutic dosage forms. Moreover, while not wishing to be bound by any particular theory, certain solid forms are characterized by physical properties (e.g., density, compressibility, hardness, morphology, cleavage, stickiness, solubility, water uptake, electrical properties, thermal behavior, solid-state reactivity, physical stability, and chemical stability) affecting particular processes (e.g., yield, filtration, washing, drying, milling, mixing, tableting, flowability, dissolution, formulation, and lyophilization) which make certain solid forms suitable for the manufacture of a solid dosage form. Such properties can be determined using particular analytical chemical techniques, including solid-state analytical techniques (e.g., X-ray diffraction, microscopy, spectroscopy, and thermal analysis), as described herein.

The identification and selection of a solid form of a pharmaceutical compound are complex, given that a change in solid form may affect a variety of physical and chemical properties, which may provide benefits or drawbacks in processing, formulation, stability, bioavailability, storage, and handling (e.g., shipping), among other important pharmaceutical characteristics. Useful pharmaceutical solids include crystalline solids and amorphous solids, depending on the product and its mode of administration. Amorphous solids are characterized by a lack of long-range structural order, whereas crystalline solids are characterized by structural periodicity. The desired class of pharmaceutical solid depends upon the specific application; amorphous solids are sometimes selected on the basis of, e.g., an enhanced dissolution profile, while crystalline solids may be desirable for properties such as, e.g., physical, or chemical stability.

Whether crystalline or amorphous, solid forms of a pharmaceutical compound include single-component and multiple-component solids. Single-component solids consist essentially of the pharmaceutical compound or active ingredient in the absence of other compounds. Variety among single-component crystalline materials may potentially arise from the phenomenon of polymorphism, wherein multiple three-dimensional arrangements exist for a particular pharmaceutical compound.

Notably, it is not possible to predict a priori if crystalline forms of a compound even exist, let alone how to successfully prepare them (see, e.g., Braga and Grepioni, 2005, “Making crystals from crystals: a green route to crystal engineering and polymorphism,” Chem. Commun.:3635-3645 (with respect to crystal engineering, if instructions are not very precise and/or if other external factors affect the process, the result can be unpredictable); Jones et al., 2006, “Pharmaceutical Cocrystals: An Emerging Approach to Physical Property Enhancement,” MRS Bulletin 31:875-879 (At present it is not generally possible to computationally predict the number of observable polymorphs of even the simplest molecules); Price, 2004, “The computational prediction of pharmaceutical crystal structures and polymorphism,” Advanced Drug Delivery Reviews 56:301-319 (“Price”); and Bernstein, 2004, “Crystal Structure Prediction and Polymorphism,” ACA Transactions 39:14-23 (a great deal still needs to be learned and done before one can state with any degree of confidence the ability to predict a crystal structure, much less polymorphic forms)).

The variety of possible solid forms creates potential diversity in physical and chemical properties for a given pharmaceutical compound. The discovery and selection of solid forms are of great importance in the development of an effective, stable, and marketable pharmaceutical product.

### Forms of Compound I

### Compound I Form I

In one embodiment, provided is crystalline 4-(((R)-1-cyanoethyl)amino)-6-(3-cyanopyrrolo[1,2-b]pyridazin-7-yl)-N—((R)-2-fluoro-3-hydroxy-3-methylbutyl)nicotinamide Form I (Compound I Form I), characterized by an X-ray powder diffractogram comprising peaks (±0.2°) at 7.5, 12.3, and 7.2°2θ as determined on a diffractometer using Cu-Kα radiation.

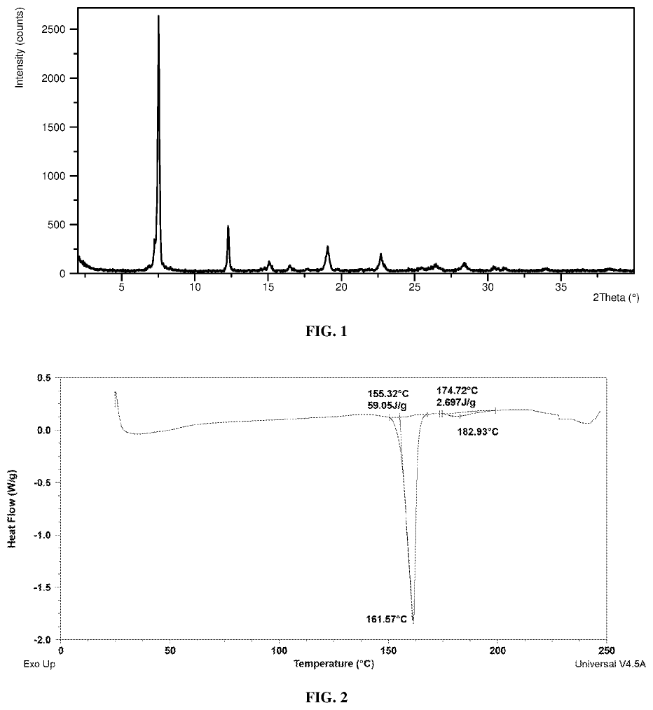

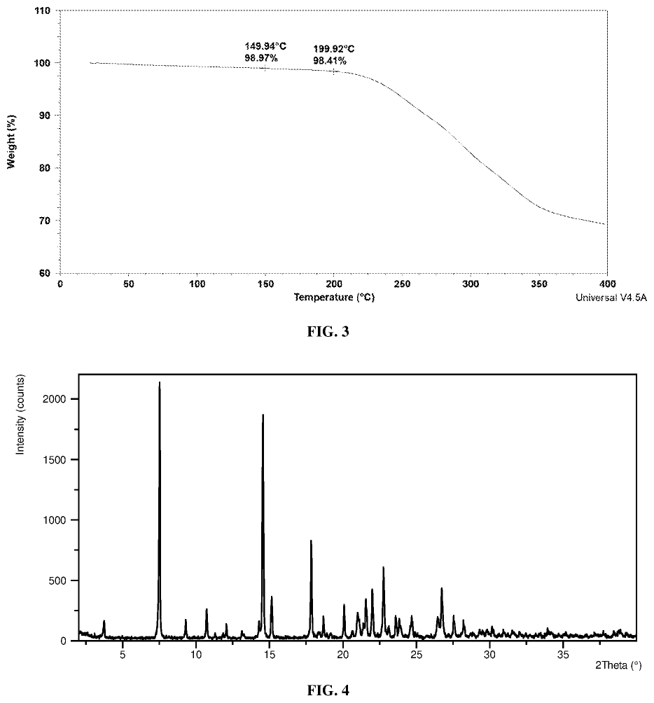

In some embodiments, Compound I Form I is further characterized by:
i) one or more peaks at 19.1°2θ±0.2°, 22.7±0.2°, or 15.1±0.2°;
ii) a diffractogram substantially as shown in FIG. 1;
iii) a differential scanning calorimetry (DSC) curve comprising an endotherm at about 155.3° C. (onset temperature) and an endotherm at about 174.7° C. (onset temperature);
iv) a differential scanning calorimetry (DSC) curve substantially as shown in FIG. 2;
v) thermogravimetric analysis (TGA) showing a weight loss of about 1.0 wt % up to about 150° C.; or
vi) thermogravimetric analysis (TGA) comprising a thermogram substantially as shown in FIG. 3.

In some embodiments, Compound I Form I is further characterized by an X-ray powder diffractogram comprising one or more additional peaks (±0.2°) at 19.1, 22.7, or 15.1°2θ as determined on a diffractometer using Cu-Kα radiation. In some embodiments, Compound I Form I is further characterized by an X-ray powder diffractogram comprising one or more additional peaks (±0.2°) at 28.4, 26.4, or 16.5°2θ as determined on a diffractometer using Cu-Kα radiation. In some embodiments, Compound I Form I is further characterized by an X-ray powder diffractogram as substantially shown in FIG. 1.

In some embodiments, Compound I Form I is further characterized by a DSC curve comprising an endotherm at about 155.3° C. (onset temperature) and an endotherm at about 174.7° C. (onset temperature). In some embodiments, Compound I Form I is further characterized by a DSC curve comprising an endotherm at about 161.6° C. (peak) and an endotherm at 182.9° C. (peak). In some embodiments, Compound I Form I is further characterized by a DSC curve as substantially shown in FIG. 2.

In some embodiments, Compound I Form I is further characterized by TGA showing a weight loss of about 1.0 wt % from ambient temperature to about 150° C. In some embodiments, Compound I Form I is further characterized by TGA comprising a thermogram substantially as shown in FIG. 3.

In some embodiments, Compound I Form I is unsolvated.

### Compound I Form II

In one embodiment, provided is crystalline 4-(((R)-1-cyanoethyl)amino)-6-(3-cyanopyrrolo[1,2-b]pyridazin-7-yl)-N—((R)-2-fluoro-3-hydroxy-3-methylbutyl)nicotinamide Form II (Compound I Form II), characterized by an X-ray powder diffractogram comprising peaks (±0.2°) at 7.5, 14.6, and 17.8°2θ as determined on a diffractometer using Cu-Kα radiation.

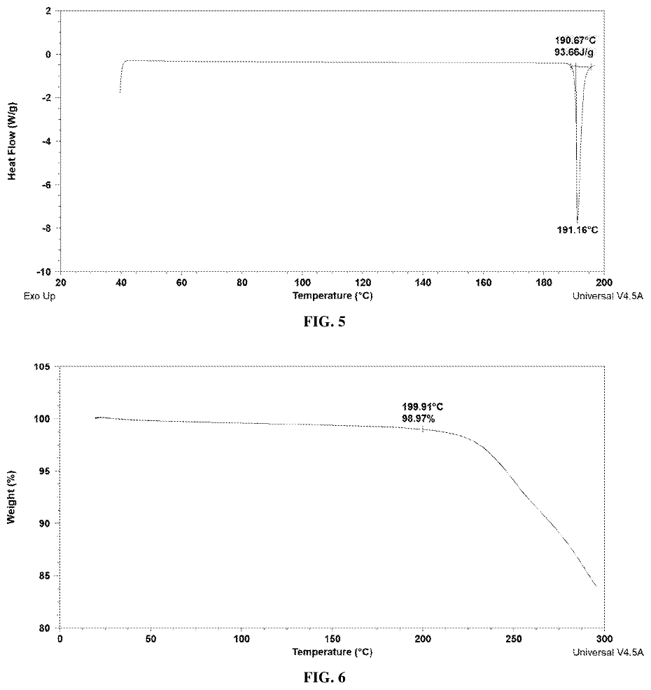

In some embodiments, Compound I Form II is further characterized by:
i) one or more peaks at 22.8°2θ±0.2°, 26.7±0.2°, or 22.0±0.2°;
ii) a diffractogram substantially as shown in FIG. 4;
iii) a differential scanning calorimetry (DSC) curve comprising an endotherm at about 190.7° C. (onset temperature);
iv) a differential scanning calorimetry (DSC) curve substantially as shown in FIG. 5;
v) thermogravimetric analysis (TGA) showing a weight loss of about 1.0 wt % up to about 200° C.; or
vi) thermogravimetric analysis (TGA) comprising a thermogram substantially as shown in FIG. 6.

In some embodiments, Compound I Form II is further characterized by an X-ray powder diffractogram comprising one or more additional peaks (±0.2°) at 22.8, 26.7, or 22.0°2θ as determined on a diffractometer using Cu-Kα radiation. In some embodiments, Compound I Form II is further characterized by an X-ray powder diffractogram comprising one or more additional peaks (±0.2°) at 15.2, 21.6, or 20.1°2θ as determined on a diffractometer using Cu-Kα radiation. In some embodiments, Compound I Form II is further characterized by an X-ray powder diffractogram as substantially shown in FIG. 4.

In some embodiments, Compound I Form II is further characterized by a DSC curve comprising an endotherm at about 190.7° C. (onset temperature). In some embodiments, Compound I Form II is further characterized by a DSC curve comprising an endotherm at about 191.2° C. (peak). In some embodiments, Compound I Form II is further characterized by a DSC curve as substantially shown in FIG. 5.

In some embodiments, Compound I Form II is further characterized by TGA showing a weight loss of about 1.0 wt % from ambient temperature to about 200° C. In some embodiments, Compound I Form II is further characterized by TGA comprising a thermogram substantially as shown in FIG. 6.

In some embodiments, Compound I Form II is unsolvated.

### Compound I Form III

In one embodiment, provided is crystalline 4-(((R)-1-cyanoethyl)amino)-6-(3-cyanopyrrolo[1,2-b]pyridazin-7-yl)-N—((R)-2-fluoro-3-hydroxy-3-methylbutyl)nicotinamide Form III (Compound I Form III), characterized by an X-ray powder diffractogram comprising peaks (±0.2°) at 21.2, 12.3, and 10.7°2θ as determined on a diffractometer using Cu-Kα radiation.

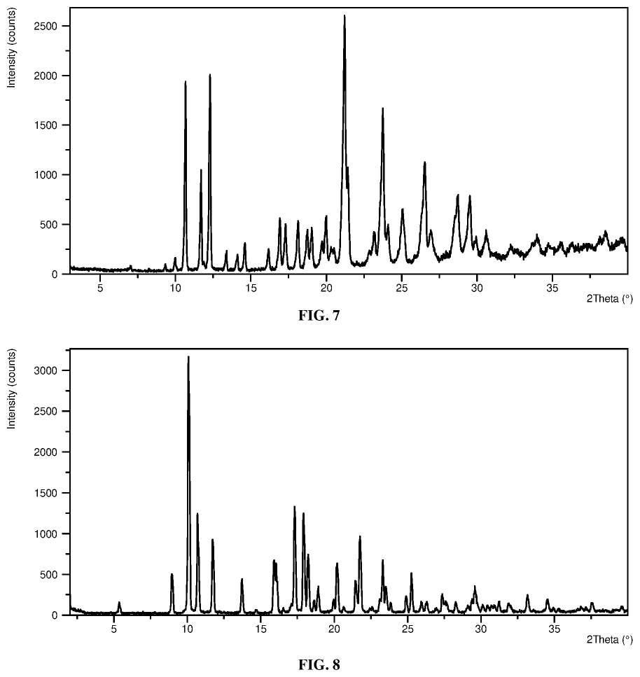

In some embodiments, Compound I Form III is further characterized by an X-ray powder diffractogram comprising one or more additional peaks (±0.2°) at 23.8, 11.7, or 21.5°2θ as determined on a diffractometer using Cu-Kα radiation. In some embodiments, Compound I Form III is further characterized by an X-ray powder diffractogram comprising one or more additional peaks (±0.2°) at 25.1, 16.9, or 20.0°2θ as determined on a diffractometer using Cu-Kα radiation. In some embodiments, Compound I Form III is further characterized by an X-ray powder diffractogram as substantially shown in FIG. 7.

In some embodiments, Compound I Form III is unsolvated.

### Compound I Form IV

In one embodiment, provided is crystalline 4-(((R)-1-cyanoethyl)amino)-6-(3-cyanopyrrolo[1,2-b]pyridazin-7-yl)-N—((R)-2-fluoro-3-hydroxy-3-methylbutyl)nicotinamide Form IV (Compound I Form IV), characterized by an X-ray powder diffractogram comprising peaks (±0.2°) at 10.1, 10.7, and 17.9°2θ as determined on a diffractometer using Cu-Kα radiation.

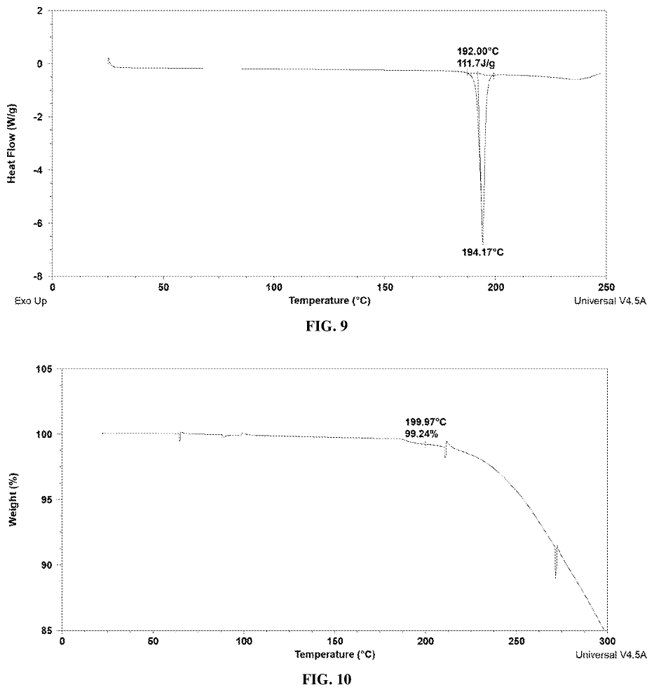

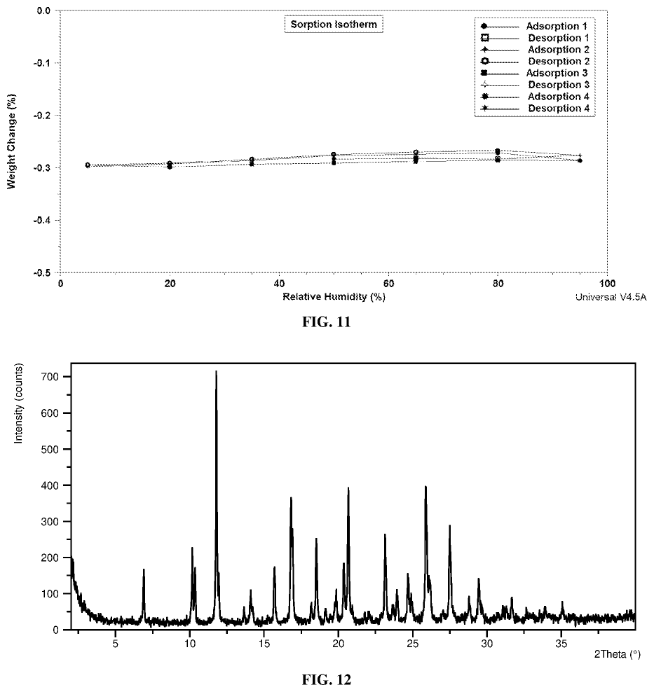

In some embodiments, Compound I Form IV is further characterized by:
i) one or more peaks at 17.3°2θ±0.2°, 11.7±0.2°, or 21.7±0.2°;
ii) a diffractogram substantially as shown in FIG. 8;
iii) a differential scanning calorimetry (DSC) curve comprising an endotherm at about 192.0° C. (onset temperature);
iv) a differential scanning calorimetry (DSC) curve substantially as shown in FIG. 9;
v) thermogravimetric analysis (TGA) showing a weight loss of about 0.8 wt % up to about 200° C.;
vi) thermogravimetric analysis (TGA) comprising a thermogram substantially as shown in FIG. 10; or
vii) a dynamic vapor sorption (DVS) curve substantially as shown in FIG. 11.

In some embodiments, Compound I Form IV is further characterized by an X-ray powder diffractogram comprising one or more additional peaks (±0.2°) at 17.3, 11.7, or 21.7°2θ as determined on a diffractometer using Cu-Kα radiation. In some embodiments, Compound I Form IV is further characterized by an X-ray powder diffractogram comprising one or more additional peaks (±0.2°) at 18.2, 23.3, or 15.9°2θ as determined on a diffractometer using Cu-Kα radiation. In some embodiments, Compound I Form IV is further characterized by an X-ray powder diffractogram as substantially shown in FIG. 8.

In some embodiments, Compound I Form IV is further characterized by a DSC curve comprising an endotherm at about 192.0° C. (onset temperature). In some embodiments, Compound I Form IV is further characterized by a DSC curve comprising an endotherm at about 194.2° C. (peak). In some embodiments, Compound I Form IV is further characterized by a DSC curve as substantially shown in FIG. 9.

In some embodiments, Compound I Form IV is further characterized by TGA showing a weight loss of about 0.8 wt % up to about 200° C. In some embodiments, Compound I Form IV is further characterized by TGA comprising a thermogram substantially as shown in FIG. 10.

In some embodiments, Compound I Form IV is further characterized by a DVS curve substantially as shown in FIG. 11.

In some embodiments, Compound I Form IV is unsolvated.

### Compound I Form V

In one embodiment, provided is crystalline 4-(((R)-1-cyanoethyl)amino)-6-(3-cyanopyrrolo[1,2-b]pyridazin-7-yl)-N—((R)-2-fluoro-3-hydroxy-3-methylbutyl)nicotinamide Form V (Compound I Form V), characterized by an X-ray powder diffractogram comprising peaks (±0.2°) at 11.8, 25.9, and 20.7°2θ as determined on a diffractometer using Cu-Kα radiation.

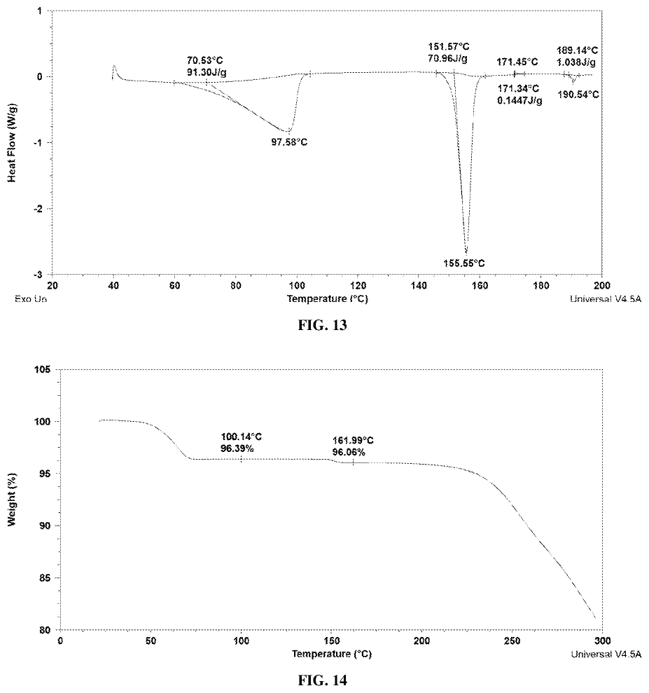

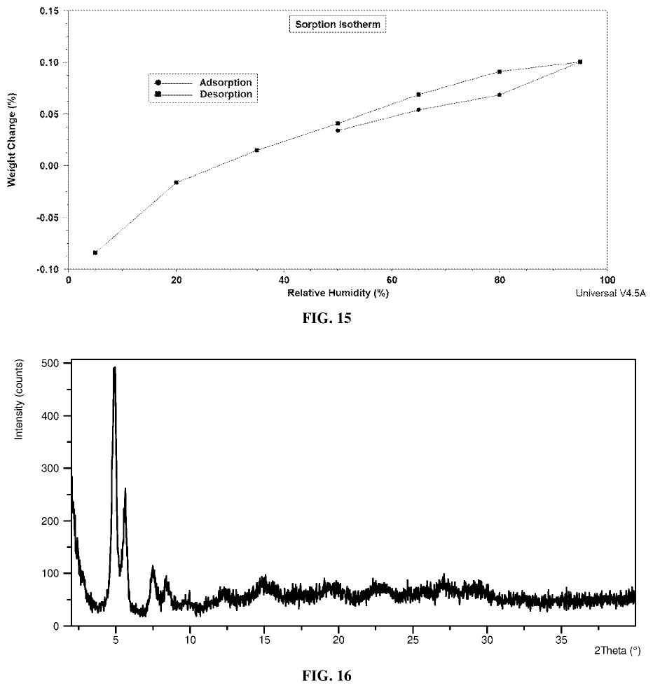

In some embodiments, Compound I Form V is further characterized by:
i) one or more peaks at 16.8°2θ±0.2°, 23.1±0.2°, or 18.5±0.2°;
ii) a diffractogram substantially as shown in FIG. 12;
iii) a differential scanning calorimetry (DSC) curve comprising an endotherm at about 70.5° C. (onset temperature), an endotherm at about 151.6° C. (onset temperature), and an endotherm at about 189.1° C. (onset temperature);
iv) a differential scanning calorimetry (DSC) curve substantially as shown in FIG. 13;
v) thermogravimetric analysis (TGA) showing a weight loss of about 3.6 wt % up to about 100° C.;
vi) thermogravimetric analysis (TGA) comprising a thermogram substantially as shown in FIG. 14; or
vii) a dynamic vapor sorption (DVS) curve substantially as shown in FIG. 15.

In some embodiments, Compound I Form V is further characterized by an X-ray powder diffractogram comprising one or more additional peaks (±0.2°) at 16.8, 23.1, or 18.5°2θ as determined on a diffractometer using Cu-Kα radiation. In some embodiments, Compound I Form V is further characterized by an X-ray powder diffractogram comprising one or more additional peaks (±0.2°) at 10.2, 20.4, or 15.7°2θ as determined on a diffractometer using Cu-Kα radiation. In some embodiments, Compound I Form V is further characterized by an X-ray powder diffractogram as substantially shown in FIG. 12.

In some embodiments, Compound I Form V is further characterized by a DSC curve comprising an endotherm at about 70.5° C. (onset temperature), an endotherm at about 151.6° C. (onset temperature), and an endotherm at about 189.1° C. (onset temperature). In some embodiments, Compound I Form V is further characterized by a DSC curve comprising an exotherm at about 171° C. (onset temperature). In some embodiments, Compound I Form V is further characterized by a DSC curve comprising an endotherm at about 97.6° C. (peak), an endotherm at 155.6° C. (peak) and an endotherm at 190.5° C. (peak). In some embodiments, Compound I Form V is further characterized by a DSC curve as substantially shown in FIG. 13.

In some embodiments, Compound I Form V is further characterized by TGA showing a weight loss of about 3.6 wt % up to about 100° C. In some embodiments, Compound I Form V is further characterized by TGA comprising a thermogram substantially as shown in FIG. 14.

In some embodiments, Compound I Form V is further characterized by a DVS curve substantially as shown in FIG. 15.

In some embodiments, Compound I Form V is a monohydrate. In some embodiments, Compound I Form V comprises about 1 mole equivalent of water.

### Compound I Form VI

In one embodiment, provided is crystalline 4-(((R)-1-cyanoethyl)amino)-6-(3-cyanopyrrolo[1,2-b]pyridazin-7-yl)-N—((R)-2-fluoro-3-hydroxy-3-methylbutyl)nicotinamide Form VI (Compound I Form VI), characterized by an X-ray powder diffractogram comprising peaks (±0.2°) at 4.9, 5.6, and 7.4°2θ as determined on a diffractometer using Cu-Kα radiation.

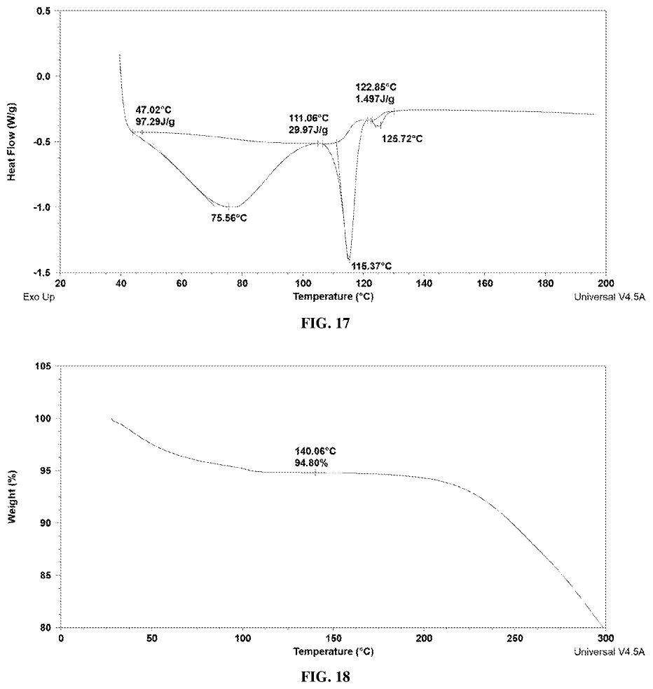

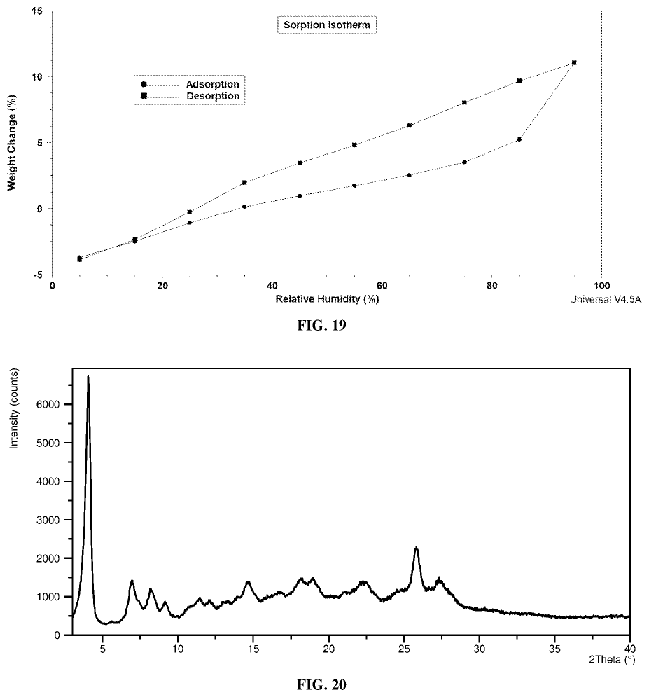

In some embodiments, Compound I Form VI is further characterized by:
i) one or more peaks at 8.4°2θ±0.2°; 12.3±0.2°; or 27.2±0.2°;
ii) a diffractogram substantially as shown in FIG. 16;
iii) a differential scanning calorimetry (DSC) curve comprising an endotherm at about 47.0° C. (onset temperature), an endotherm at about 111.1° C. (onset temperature), and an endotherm at about 122.9° C. (onset temperature);
iii) a differential scanning calorimetry (DSC) curve substantially as shown in FIG. 17;
v) thermogravimetric analysis (TGA) showing a weight loss of about 5.2 wt % up to about 140° C.;
vi) thermogravimetric analysis (TGA) comprising a thermogram substantially as shown in FIG. 18; or
vii) a dynamic vapor sorption (DVS) curve substantially as shown in FIG. 19.

In some embodiments, Compound I Form VI is further characterized by an X-ray powder diffractogram comprising one or more additional peaks (±0.2°) at 8.4, 12.3, or 27.2°2θ as determined on a diffractometer using Cu-Kα radiation. In some embodiments, Compound I Form VI is further characterized by an X-ray powder diffractogram as substantially shown in FIG. 16.

In some embodiments, Compound I Form VI is further characterized by a DSC curve comprising an endotherm at about 47.0° C. (onset temperature), an endotherm at about 111.1° C. (onset temperature), and an endotherm at about 122.9° C. (onset temperature). In some embodiments, Compound I Form VI is further characterized by a DSC curve comprising an endotherm at about 75.6° C. (peak), an endotherm at 115.4° C. (peak), and an endotherm at 125.7° C. (peak). In some embodiments, Compound I Form VI is further characterized by a DSC curve as substantially shown in FIG. 17.

In some embodiments, Compound I Form VI is further characterized by TGA showing a weight loss of about 5.2 wt % from ambient temperature to about 140° C. In some embodiments, Compound I Form VI is further characterized by TGA comprising a thermogram substantially as shown in FIG. 18.

In some embodiments, Compound I Form VI is further characterized by a DVS curve substantially as shown in FIG. 19.

In some embodiments, Compound I Form VI is a hydrate. In some embodiments, Compound I Form VI comprises about 1.25 mole equivalents of water.

### Compound I Form VII

In one embodiment, provided is crystalline 4-(((R)-1-cyanoethyl)amino)-6-(3-cyanopyrrolo[1,2-b]pyridazin-7-yl)-N—((R)-2-fluoro-3-hydroxy-3-methylbutyl)nicotinamide Form VII (Compound I Form VII), characterized by an X-ray powder diffractogram comprising peaks (±0.2°) at 4.0, 25.8, and 6.9°2θ as determined on a diffractometer using Cu-Kα radiation.

In some embodiments, Compound I Form VII is further characterized by an X-ray powder diffractogram comprising one or more additional peaks (±0.2°) at 8.2, 14.7, or 18.1°2θ as determined on a diffractometer using Cu-Kα radiation. In some embodiments, Compound I Form VII is further characterized by an X-ray powder diffractogram comprising one or more additional peaks (±0.2°) at 18.9, 11.4, or 9.1°2θ as determined on a diffractometer using Cu-Kα radiation. In some embodiments, Compound I Form VII is further characterized by an X-ray powder diffractogram as substantially shown in FIG. 20.

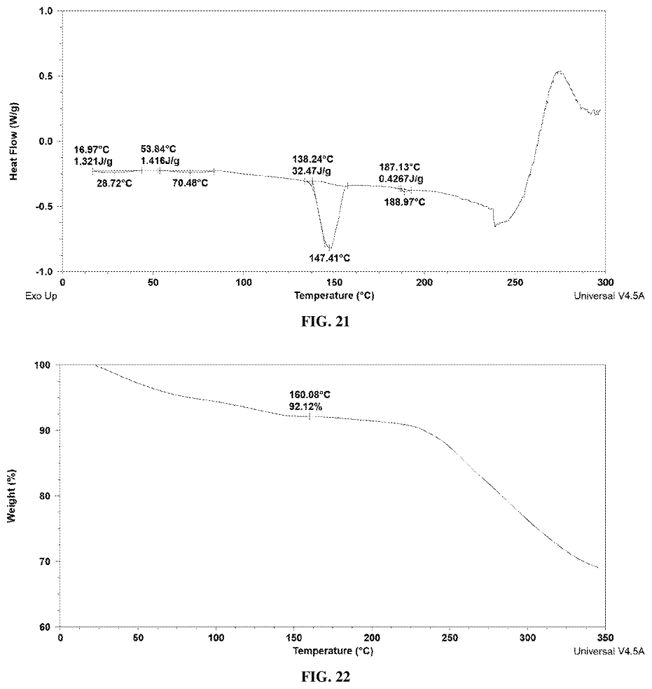

In some embodiments, Compound I Form VII is further characterized by a DSC curve comprising an endotherm at about 17° C. (onset temperature), an endotherm at about 53.8° C. (onset temperature), an endotherm at about 138.2° C. (onset temperature), and an endotherm at about 187.1° C. (onset temperature). In some embodiments, Compound I Form VII is further characterized by a DSC curve comprising an endotherm at about 28.7° C. (peak), an endotherm at about 70.5° C. (peak), an endotherm at about 147.4° C. (peak) and an endotherm at about 189.0° C. (peak). In some embodiments, Compound I Form VII is further characterized by a DSC curve as substantially shown in FIG. 21.

In some embodiments, Compound I Form VII is further characterized by TGA showing a weight loss of about 7.9 wt % from ambient to about 160° C. In some embodiments, Compound I Form VII is further characterized by TGA comprising a thermogram substantially as shown in FIG. 22.

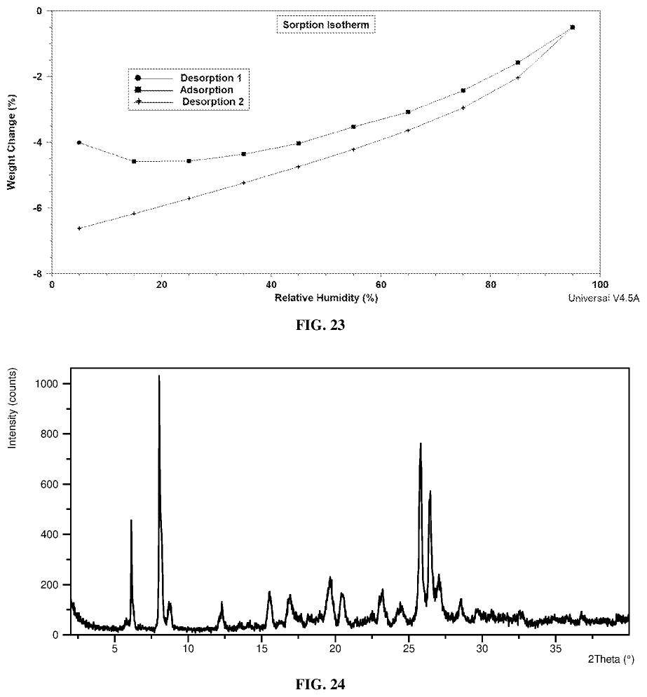

In some embodiments, Compound I Form VII is further characterized by a DVS curve substantially as shown in FIG. 23.

In some embodiments, Compound I Form VII is a water:THF solvate.

### Compound I Form VIII

In one embodiment, provided is crystalline 4-(((R)-1-cyanoethyl)amino)-6-(3-cyanopyrrolo[1,2-b]pyridazin-7-yl)-N—((R)-2-fluoro-3-hydroxy-3-methylbutyl)nicotinamide Form VIII (Compound I Form VIII), characterized by an X-ray powder diffractogram comprising peaks (±0.2°) at 8.0, 25.8, and 6.1°2θ as determined on a diffractometer using Cu-Kα radiation.

In some embodiments, Compound I Form VIII is further characterized by an X-ray powder diffractogram comprising one or more additional peaks (±0.2°) at 19.7, 23.2, or 15.5°2θ as determined on a diffractometer using Cu-Kα radiation. In some embodiments, Compound I Form VIII is further characterized by an X-ray powder diffractogram comprising one or more additional peaks (±0.2°) at 16.9, 20.4, or 12.3°2θ as determined on a diffractometer using Cu-Kα radiation. In some embodiments, Compound I Form VIII is further characterized by an X-ray powder diffractogram as substantially shown in FIG. 24.

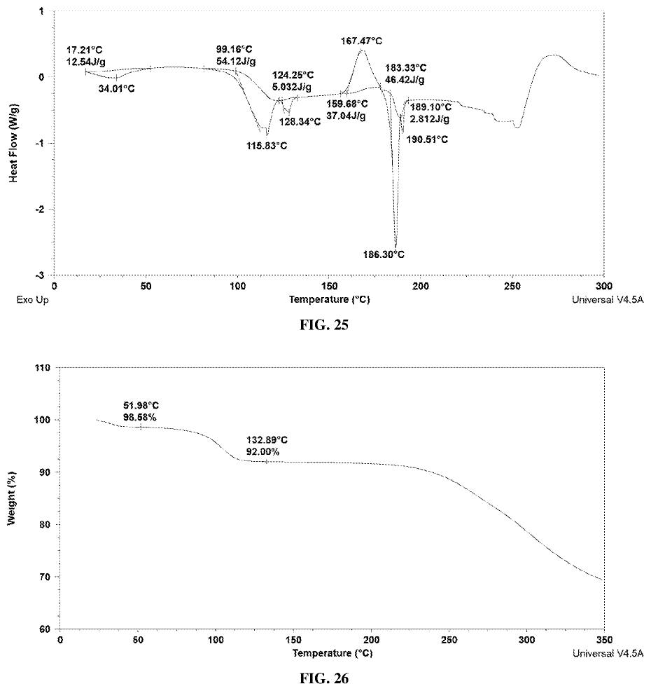

In some embodiments, Compound I Form VIII is further characterized by a DSC curve comprising an endotherm at about 17.2° C. (onset temperature), an endotherm at about 99.2° C. (onset temperature), an endotherm at about 124.3° C. (onset temperature), and an endotherm at about 183.3° C. (onset temperature). In some embodiments, Compound I Form VIII is further characterized by a DSC curve comprising an endotherm at about 34.0° C. (peak), an endotherm at about 115.8° C. (peak), an endotherm at about 128.3° C. (peak) and an endotherm at about 186.3° C. (peak). In some embodiments, Compound I Form VIII is further characterized by a DSC curve as substantially shown in FIG. 25.

In some embodiments, Compound I Form VIII is further characterized by TGA showing a weight loss of about 1.4 wt % from ambient temperature to about 52° C. and a weight loss of about 6.6 wt % from about 52° C. to about 160° C. In some embodiments, Compound I Form VIII is further characterized by TGA comprising a thermogram substantially as shown in FIG. 26.

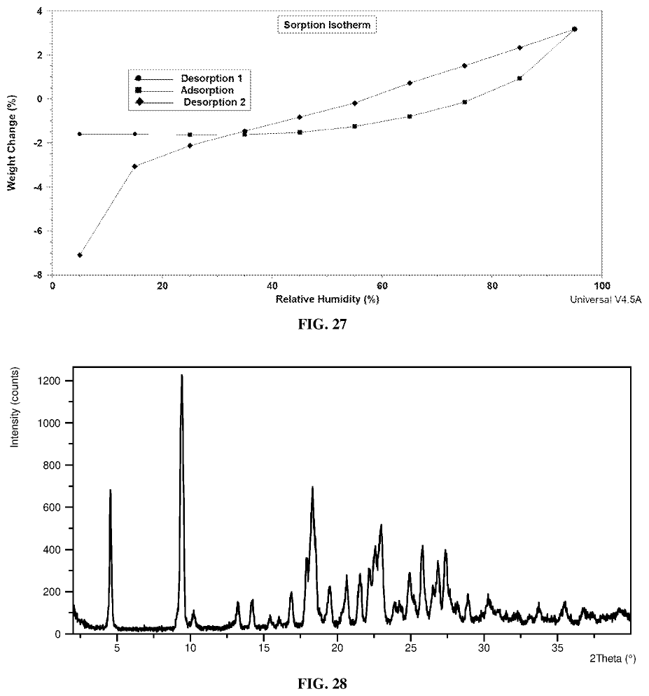

In some embodiments, Compound I Form VIII is further characterized by a DVS curve substantially as shown in FIG. 27.

In some embodiments, Compound I Form VIII is a water:ACN solvate.

### Compound I Form IX

In one embodiment, provided is crystalline 4-(((R)-1-cyanoethyl)amino)-6-(3-cyanopyrrolo[1,2-b]pyridazin-7-yl)-N—((R)-2-fluoro-3-hydroxy-3-methylbutyl)nicotinamide Form IX (Compound I Form IX), characterized by an X-ray powder diffractogram comprising peaks (±0.2°) at 9.4, 4.5, and 18.3°2θ as determined on a diffractometer using Cu-Kα radiation.

In some embodiments, Compound I Form IX is further characterized by an X-ray powder diffractogram comprising one or more additional peaks (±0.2°) at 23.0, 22.6, or 25.8°2θ as determined on a diffractometer using Cu-Kα radiation. In some embodiments, Compound I Form IX is further characterized by an X-ray powder diffractogram comprising one or more additional peaks (±0.2°) at 17.9, 24.9, or 20.7°2θ as determined on a diffractometer using Cu-Kα radiation. In some embodiments, Compound I Form IX is further characterized by an X-ray powder diffractogram as substantially shown in FIG. 28.

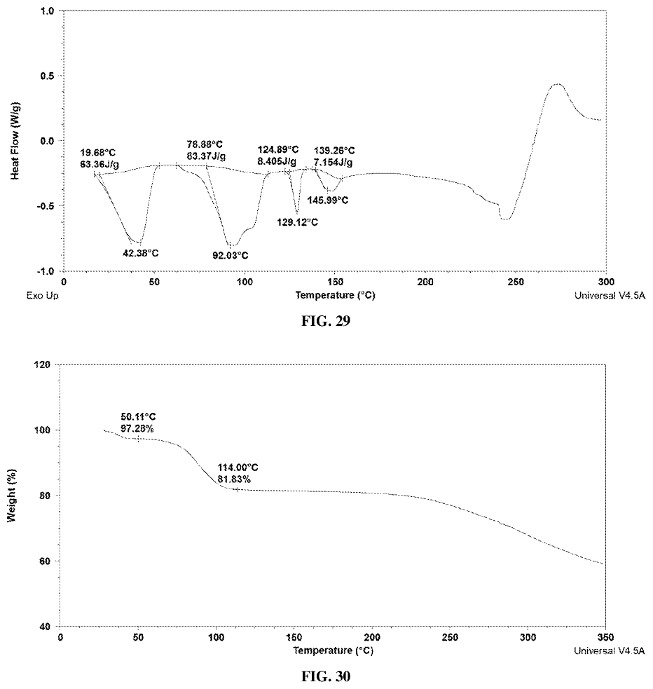

In some embodiments, Compound I Form IX is further characterized by a DSC curve comprising an endotherm at about 19.7° C. (onset temperature), an endotherm at about 78.9° C. (onset temperature), an endotherm at about 124.9° C. (onset temperature), and an endotherm at about 139.3° C. (onset temperature). In some embodiments, Compound I Form IX is further characterized by a DSC curve comprising an endotherm at about 42.4° C. (peak), endotherm at about 92.0° C. (peak), endotherm at about 129.1° C. (peak) and endotherm at about 146.0° C. (peak). In some embodiments, Compound I Form IX is further characterized by a DSC curve as substantially shown in FIG. 29.

In some embodiments, Compound I Form IX is further characterized by TGA showing a weight loss of about 2.7 wt % from ambient temperature to about 50° C. and a weight loss of about 15.5 wt % from about 50° C. to about 114° C. In some embodiments, Compound I Form IX is further characterized by TGA comprising a thermogram substantially as shown in FIG. 30.

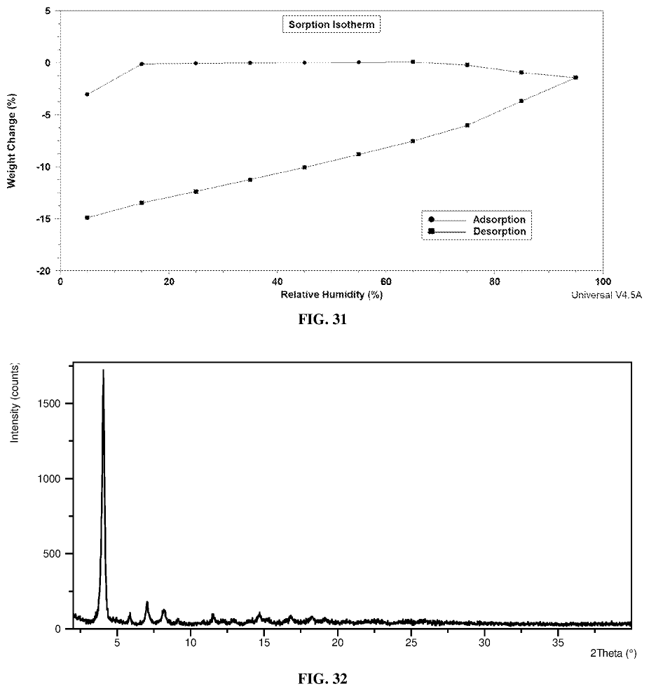

In some embodiments, Compound I Form IX is further characterized by a DVS curve substantially as shown in FIG. 31.

In some embodiments, Compound I Form IX is a water:2-MeTHF solvate.

### Compound I Form X

In one embodiment, provided is crystalline 4-(((R)-1-cyanoethyl)amino)-6-(3-cyanopyrrolo[1,2-b]pyridazin-7-yl)-N—((R)-2-fluoro-3-hydroxy-3-methylbutyl)nicotinamide Form X (Compound I Form X), characterized by an X-ray powder diffractogram comprising peaks (±0.2°) at 4.0, 7.0, and 8.2°2θ as determined on a diffractometer using Cu-Kα radiation.

In some embodiments, Compound I Form X is further characterized by an X-ray powder diffractogram comprising one or more additional peaks (±0.2°) at 5.9, 14.7, or 11.5°2θ as determined on a diffractometer using Cu-Kα radiation. In some embodiments, Compound I Form X is further characterized by an X-ray powder diffractogram comprising one or more additional peaks (±0.2°) at 16.8, 18.3, or 9.1°2θ as determined on a diffractometer using Cu-Kα radiation. In some embodiments, Compound I Form X is further characterized by an X-ray powder diffractogram as substantially shown in FIG. 32.

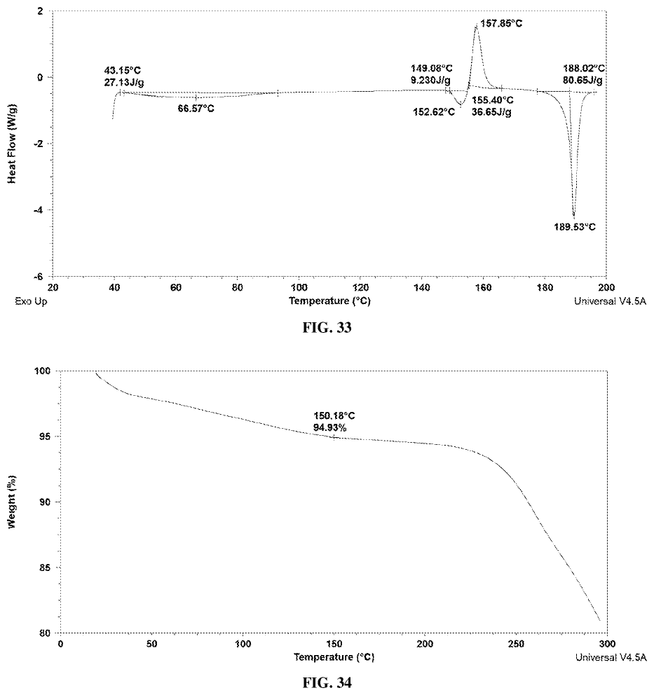

In some embodiments, Compound I Form X is further characterized by a DSC curve comprising an endotherm at about 43.2° C. (onset temperature), an endotherm at about 149.1° C. (onset temperature), and an endotherm at about 188.0° C. (onset temperature). In some embodiments, Compound I Form X is further characterized by a DSC curve comprising an endotherm at about 66.6° C. (peak), an endotherm at about 152.6° C. (peak), and an endotherm at about 189.5° C. (peak). In some embodiments, Compound I Form X is further characterized by a DSC curve as substantially shown in FIG. 33.

In some embodiments, Compound I Form X is further characterized by TGA showing a weight loss of about 5.1 wt % from ambient temperature to about 150° C. In some embodiments, Compound I Form X is further characterized by TGA comprising a thermogram substantially as shown in FIG. 34.

In some embodiments, Compound I Form X is solvated. In some embodiments, Compound I Form X is an isostructural solvate. In some embodiments, Compound I Form X is an IPA, acetone, or MTBE solvate. In some embodiments, Compound I Form X is an IPA solvate. In some embodiments, Compound I Form X an acetone solvate. In some embodiments, Compound I Form X is an MTBE solvate.

### Compound I Form XI

In one embodiment, provided is crystalline 4-(((R)-1-cyanoethyl)amino)-6-(3-cyanopyrrolo[1,2-b]pyridazin-7-yl)-N—((R)-2-fluoro-3-hydroxy-3-methylbutyl)nicotinamide Form XI (Compound I Form XI), characterized by an X-ray powder diffractogram comprising peaks (±0.2°) at 4.0, 6.9, and 14.5°2θ as determined on a diffractometer using Cu-Kα radiation.

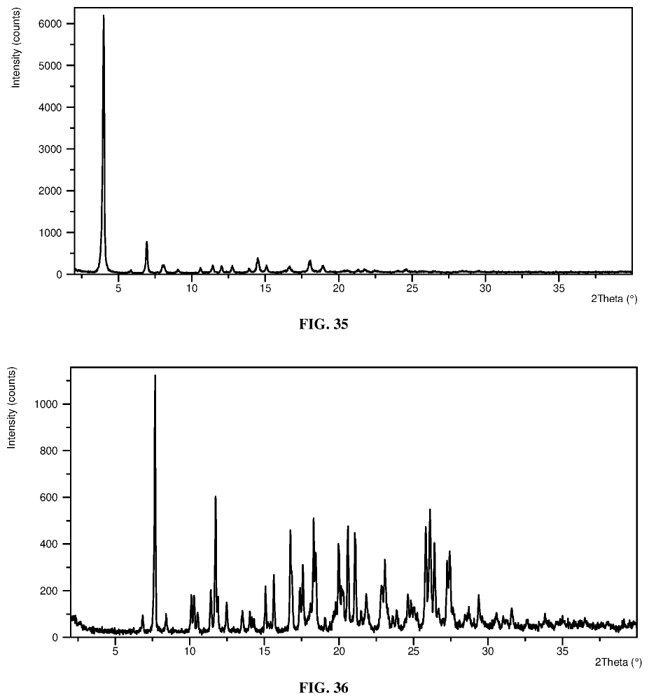

In some embodiments, Compound I Form XI is further characterized by an X-ray powder diffractogram comprising one or more additional peaks (±0.2°) at 18.0, 11.4, or 8.0°2θ as determined on a diffractometer using Cu-Kα radiation. In some embodiments, Compound I Form XI is further characterized by an X-ray powder diffractogram comprising one or more additional peaks (±0.2°) at 12.0, 15.1, or 18.9°2θ as determined on a diffractometer using Cu-Kα radiation. In some embodiments, Compound I Form XI is further characterized by an X-ray powder diffractogram as substantially shown in FIG. 35.

In some embodiments, Compound I Form XI is solvated. In some embodiments, Compound I Form XI is an isostructural solvate. In some embodiments, Compound I Form XI is a MIBK or THF solvate. In some embodiments, Compound I Form XI is a MIBK solvate. In some embodiments, Compound I Form XI is a THF solvate.

### Compound I Form XII

In one embodiment, provided is crystalline 4-(((R)-1-cyanoethyl)amino)-6-(3-cyanopyrrolo[1,2-b]pyridazin-7-yl)-N—((R)-2-fluoro-3-hydroxy-3-methylbutyl)nicotinamide Form XII (Compound I Form XII), characterized by an X-ray powder diffractogram comprising peaks (±0.2°) at 7.7, 11.7, and 26.1°2θ as determined on a diffractometer using Cu-Kα radiation.

In some embodiments, Compound I Form XII is further characterized by an X-ray powder diffractogram comprising one or more additional peaks (±0.2°) at 18.3, 16.7, or 20.6°2θ as determined on a diffractometer using Cu-Kα radiation. In some embodiments, Compound I Form XII is further characterized by an X-ray powder diffractogram comprising one or more additional peaks (±0.2°) at 21.1, 20.0, or 23.1°2θ as determined on a diffractometer using Cu-Kα radiation. In some embodiments, Compound I Form XII is further characterized by an X-ray powder diffractogram as substantially shown in FIG. 36.

### Compound I Form XIII

In one embodiment, provided is crystalline 4-(((R)-1-cyanoethyl)amino)-6-(3-cyanopyrrolo[1,2-b]pyridazin-7-yl)-N—((R)-2-fluoro-3-hydroxy-3-methylbutyl)nicotinamide Form XIII (Compound I Form XIII), characterized by an X-ray powder diffractogram comprising peaks (±0.2°) at 7.6, 14.6, and 17.9°2θ as determined on a diffractometer using Cu-Kα radiation.

In some embodiments, Compound I Form XIII is further characterized by an X-ray powder diffractogram comprising one or more additional peaks (±0.2°) at 22.8, 15.2, or 22.1°2θ as determined on a diffractometer using Cu-Kα radiation. In some embodiments, Compound I Form XIII is further characterized by an X-ray powder diffractogram comprising one or more additional peaks (±0.2°) at 21.1, 20.1, or 24.8°2θ as determined on a diffractometer using Cu-Kα radiation. In some embodiments, Compound I Form XIII is further characterized by an X-ray powder diffractogram as substantially shown in FIG. 37.

In some embodiments, Compound I Form XIII is unsolvated.

### Compound I Amorphous Form

In one embodiment, provided is amorphous 4-(((R)-1-cyanoethyl)amino)-6-(3-cyanopyrrolo[1,2-b]pyridazin-7-yl)-N—((R)-2-fluoro-3-hydroxy-3-methylbutyl)nicotinamide (Compound I amorphous Form).

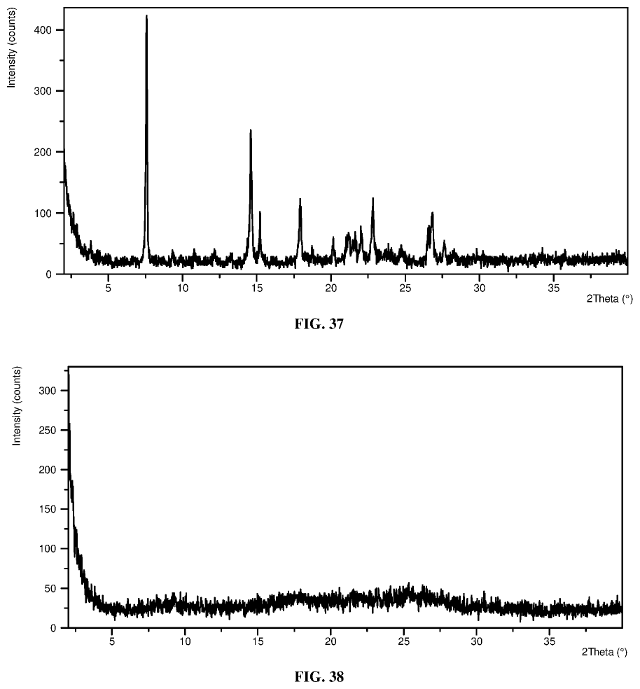

In some embodiments, Compound I amorphous Form is characterized by an X-ray powder diffractogram as substantially shown in FIG. 38.

### Compound I Mono-Citrate Form I

In one embodiment, provided is crystalline 4-(((R)-1-cyanoethyl)amino)-6-(3-cyanopyrrolo[1,2-b]pyridazin-7-yl)-N—((R)-2-fluoro-3-hydroxy-3-methylbutyl)nicotinamide mono-citrate salt Form I (Compound I mono-citrate Form I), characterized by an X-ray powder diffractogram comprising peaks (±0.2°) at 5.7, 7.0, and 22.7°2θ as determined on a diffractometer using Cu-Kα radiation.

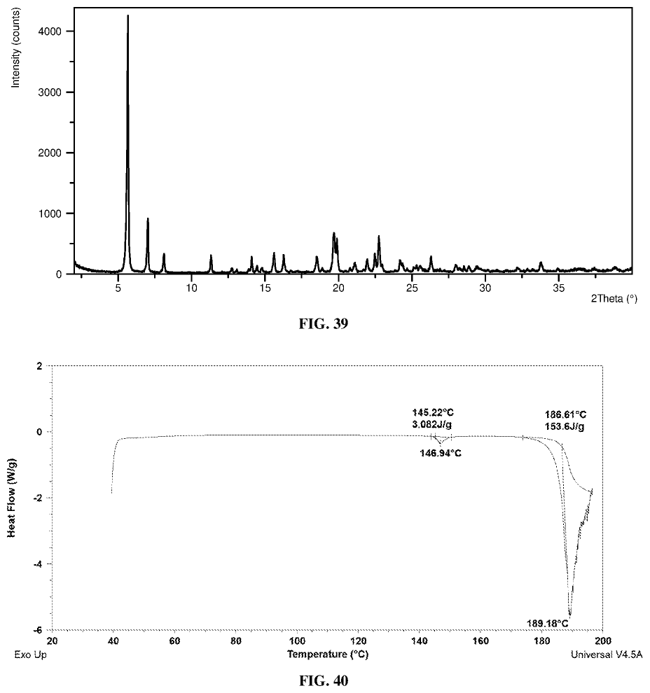

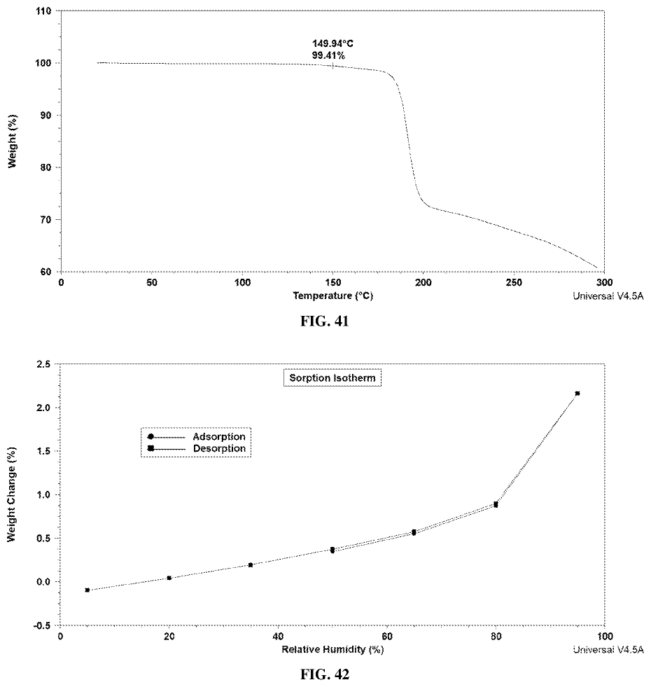

In some embodiments, Compound I mono-citrate Form I is further characterized by:
i) one or more peaks at 19.6°2θ±0.2°, 15.6±0.2°, or 8.1±0.2°;
ii) a diffractogram substantially as shown in FIG. 39;
iii) a differential scanning calorimetry (DSC) curve comprising an endotherm at about 145.2° C. (onset temperature) and an endotherm at about 186.6° C. (onset temperature);
iv) a differential scanning calorimetry (DSC) curve substantially as shown in FIG. 40;
v) thermogravimetric analysis (TGA) showing a weight loss of about 0.6 wt % up to about 150° C.;
vi) thermogravimetric analysis (TGA) comprising a thermogram substantially as shown in FIG. 41;
vii) a dynamic vapor sorption (DVS) curve substantially as shown in FIG. 42.

In some embodiments, Compound I mono-citrate Form I is further characterized by an X-ray powder diffractogram comprising one or more additional peaks (±0.2°) at 19.6, 15.6, or 8.1°2θ as determined on a diffractometer using Cu-Kα radiation. In some embodiments, Compound I mono-citrate Form I is further characterized by an X-ray powder diffractogram comprising one or more additional peaks (±0.2°) at 16.3, 11.3, or 18.5°2θ as determined on a diffractometer using Cu-Kα radiation. In some embodiments, Compound I mono-citrate Form I is further characterized by an X-ray powder diffractogram as substantially shown in FIG. 39.

In some embodiments, Compound I mono-citrate Form I is further characterized by a DSC curve comprising an endotherm at about 145.2° C. (onset temperature) and an endotherm at about 186.6° C. (onset temperature). In some embodiments, Compound I mono-citrate Form I is further characterized by a DSC curve comprising an endotherm at about 146.9° C. (peak) and an endotherm at about 189.2° C. (peak). In some embodiments, Compound I mono-citrate Form I is further characterized by a DSC curve as substantially shown in FIG. 40.

In some embodiments, Compound I mono-citrate Form I is further characterized by TGA showing a weight loss of about 0.6 wt % from ambient temperature to about 150° C. In some embodiments, Compound I mono-citrate Form I is further characterized by TGA comprising a thermogram substantially as shown in FIG. 41.

In some embodiments, Compound I mono-citrate Form I is further characterized by a DVS curve substantially as shown in FIG. 42.

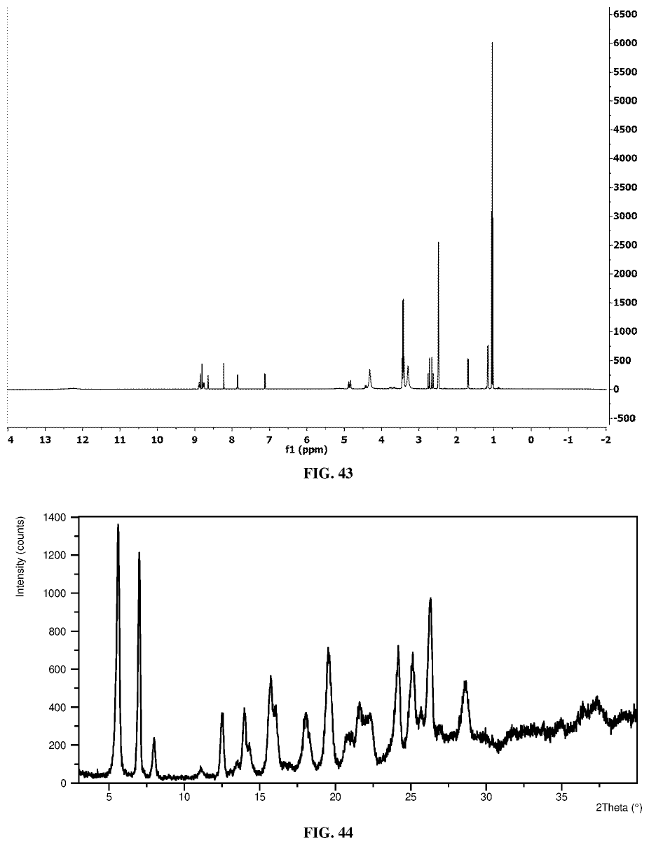

In some embodiments, Compound I mono-citrate Form I is further characterized by a H NMR spectrum substantially as shown in FIG. 43.

In some embodiments, Compound I mono-citrate Form I comprises about 1 mole equivalent of citric acid. In some embodiments, Compound I mono-citrate Form I is unsolvated.

### Compound I Mono-Citrate Form II

In one embodiment, provided is crystalline 4-(((R)-1-cyanoethyl)amino)-6-(3-cyanopyrrolo[1,2-b]pyridazin-7-yl)-N—((R)-2-fluoro-3-hydroxy-3-methylbutyl)nicotinamide mono-citrate salt Form II (Compound I mono-citrate Form II), characterized by an X-ray powder diffractogram comprising peaks (±0.2°) at 5.6, 7.0, and 24.2°2θ as determined on a diffractometer using Cu-Kα radiation.

In some embodiments, Compound I mono-citrate Form II is further characterized by an X-ray powder diffractogram comprising one or more additional peaks (±0.2°) at 19.5, 15.7, or 12.5°2θ as determined on a diffractometer using Cu-Kα radiation. In some embodiments, Compound I mono-citrate Form II is further characterized by an X-ray powder diffractogram comprising one or more additional peaks (±0.2°) at 25.1, 14.0, or 18.0°2θ as determined on a diffractometer using Cu-Kα radiation. In some embodiments, Compound I mono-citrate Form II is further characterized by an X-ray powder diffractogram as substantially shown in FIG. 44.

In some embodiments, Compound I mono-citrate Form II is unsolvated.

### Compound I Mono-Citrate Form III

In one embodiment, provided is crystalline 4-(((R)-1-cyanoethyl)amino)-6-(3-cyanopyrrolo[1,2-b]pyridazin-7-yl)-N—((R)-2-fluoro-3-hydroxy-3-methylbutyl)nicotinamide mono-citrate salt Form III (Compound I mono-citrate Form III), characterized by an X-ray powder diffractogram comprising peaks (±0.2°) at 6.3, 18.8, and 7.7°2θ as determined on a diffractometer using Cu-Kα radiation.

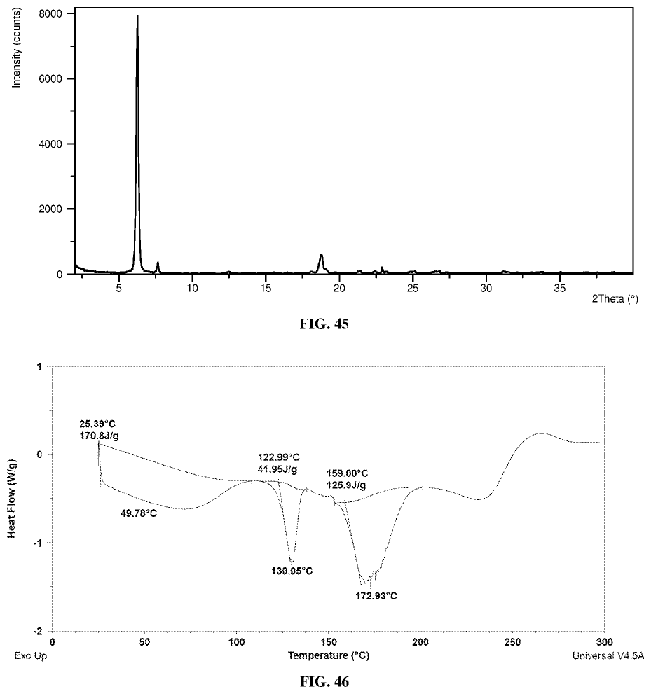

In some embodiments, Compound I mono-citrate Form III is further characterized by an X-ray powder diffractogram comprising one or more additional peaks (±0.2°) at 22.9, 19.1, or 22.4°2θ as determined on a diffractometer using Cu-Kα radiation. In some embodiments, Compound I mono-citrate Form III is further characterized by an X-ray powder diffractogram comprising one or more additional peaks (±0.2°) at 21.4, 18.1, or 25.1°2θ as determined on a diffractometer using Cu-Kα radiation. In some embodiments, Compound I mono-citrate Form III is further characterized by an X-ray powder diffractogram as substantially shown in FIG. 45.

In some embodiments, Compound I mono-citrate Form III is further characterized by a DSC curve comprising an endotherm at about 25.4° C. (onset temperature), an endotherm at about 123.0° C. (onset temperature), and an endotherm at about 159.0° C. (onset temperature). In some embodiments, Compound I mono-citrate Form III is further characterized by a DSC curve comprising an endotherm at about 49.8° C. (peak), an endotherm at about 130.1° C. (peak), and an endotherm at about 172.9° C. (peak). In some embodiments, Compound I mono-citrate Form III is further characterized by a DSC curve as substantially shown in FIG. 46.

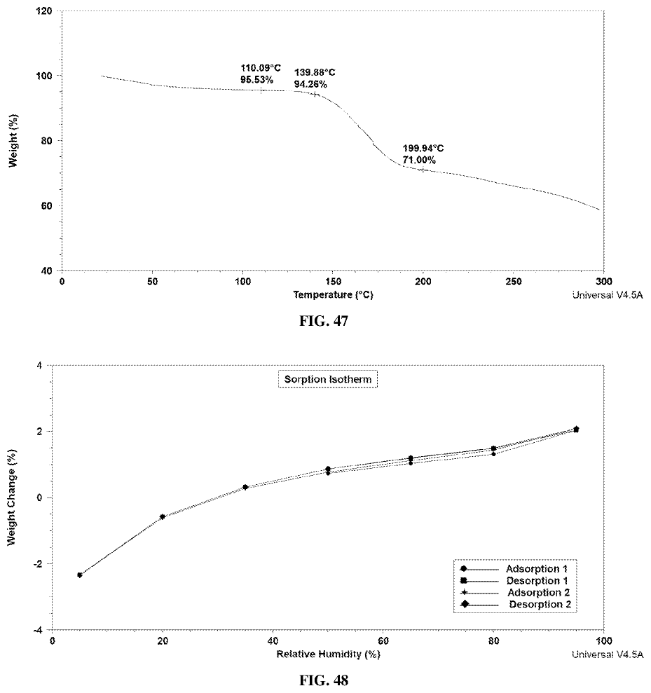

In some embodiments, Compound I mono-citrate Form III is further characterized by TGA showing a weight loss of about 4.5 wt %. In some embodiments, Compound I mono-citrate Form III is further characterized by TGA comprising a thermogram substantially as shown in FIG. 47.

In some embodiments, Compound I mono-citrate Form III is further characterized by a DVS curve substantially as shown in FIG. 48.

In some embodiments, Compound I mono-citrate Form III is a hydrate. In some embodiments, Compound I mono-citrate Form III comprises about 1.5 mole equivalent of water.

### Compound I Mono-Citrate Form IV

In one embodiment, provided is crystalline 4-(((R)-1-cyanoethyl)amino)-6-(3-cyanopyrrolo[1,2-b]pyridazin-7-yl)-N—((R)-2-fluoro-3-hydroxy-3-methylbutyl)nicotinamide mono-citrate salt Form IV (Compound I mono-citrate Form IV), characterized by an X-ray powder diffractogram comprising peaks (±0.2°) at 26.8, 25.7, and 25.1°2θ as determined on a diffractometer using Cu-Kα radiation.

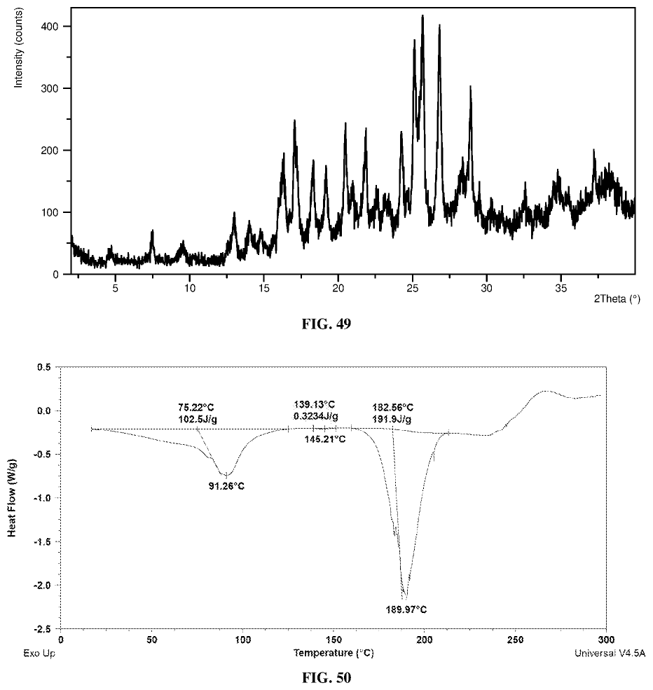

In some embodiments, Compound I mono-citrate Form IV is further characterized by an X-ray powder diffractogram comprising one or more additional peaks (±0.2°) at 17.1, 20.5, or 21.9°2θ as determined on a diffractometer using Cu-Kα radiation. In some embodiments, Compound I mono-citrate Form IV is further characterized by an X-ray powder diffractogram comprising one or more additional peaks (±0.2°) at 16.3, 13.0, or 7.4°2θ as determined on a diffractometer using Cu-Kα radiation. In some embodiments, Compound I mono-citrate Form IV is further characterized by an X-ray powder diffractogram as substantially shown in FIG. 49.

In some embodiments, Compound I mono-citrate Form IV is further characterized by a DSC curve comprising an endotherm at about 75.2° C. (onset temperature), an endotherm at about 139.1° C. (onset temperature), and an endotherm at about 182.6° C. (onset temperature). In some embodiments, Compound I mono-citrate Form IV is further characterized by a DSC curve comprising an endotherm at about 91.3° C. (peak) and an endotherm at about 190.0° C. (peak). In some embodiments, Compound I mono-citrate Form IV is further characterized by a DSC curve as substantially shown in FIG. 50.

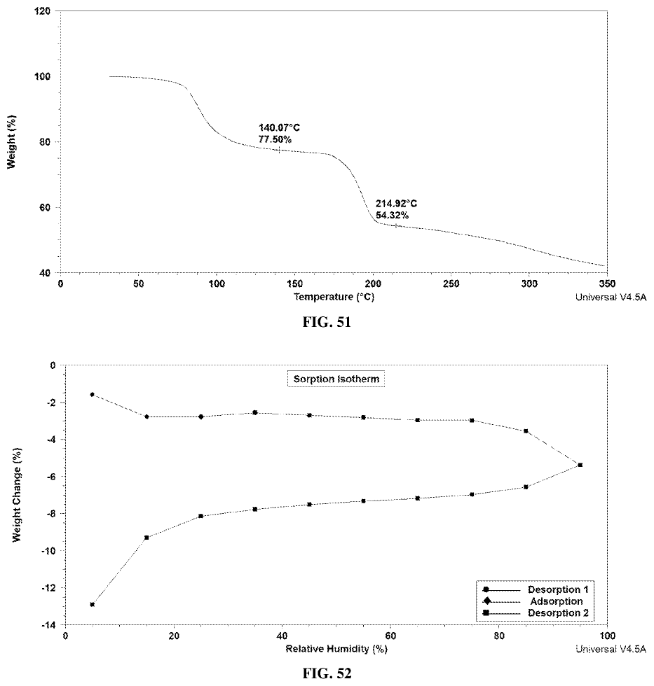

In some embodiments, Compound I mono-citrate Form IV is further characterized by TGA showing a weight loss of about 22.5 wt % from ambient temperature to about 140° C. In some embodiments, Compound I mono-citrate Form IV is further characterized by TGA comprising a thermogram substantially as shown in FIG. 51.

In some embodiments, Compound I mono-citrate Form IV is further characterized by a DVS curve substantially as shown in FIG. 52.

In some embodiments, Compound I mono-citrate Form IV is a solvate. In some embodiments, Compound I mono-citrate Form IV is a mono-hexafluoro-2-propanol solvate.

### Compound I Hemi-Citrate Form I

In one embodiment, provided is crystalline 4-(((R)-1-cyanoethyl)amino)-6-(3-cyanopyrrolo[1,2-b]pyridazin-7-yl)-N—((R)-2-fluoro-3-hydroxy-3-methylbutyl)nicotinamide hemi-citrate salt Form I (Compound I hemi-citrate Form I), characterized by an X-ray powder diffractogram comprising peaks (±0.2°) at 6.1, 7.4, and 17.1°2θ as determined on a diffractometer using Cu-Kα radiation.

In some embodiments, Compound I hemi-citrate Form I is further characterized by an X-ray powder diffractogram comprising one or more additional peaks (±0.2°) at 11.2, 5.7, or 18.0°2θ as determined on a diffractometer using Cu-Kα radiation. In some embodiments, Compound I hemi-citrate Form I is further characterized by an X-ray powder diffractogram comprising one or more additional peaks (±0.2°) at 14.9, 3.7, or 23.2°2θ as determined on a diffractometer using Cu-Kα radiation. In some embodiments, Compound I hemi-citrate Form I is further characterized by an X-ray powder diffractogram as substantially shown in FIG. 53.

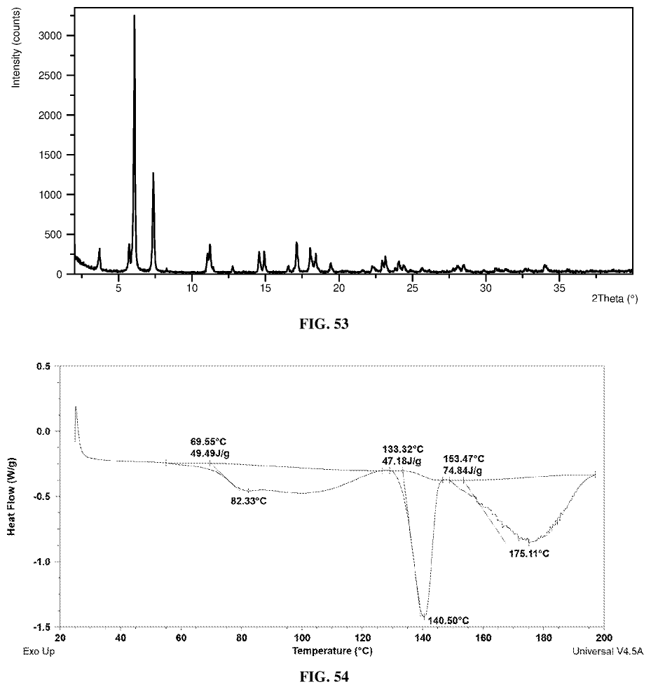

In some embodiments, Compound I hemi-citrate Form I is further characterized by a DSC curve comprising an endotherm at about 69.6° C. (onset temperature), an endotherm at about 133.3° C. (onset temperature), and an endotherm at about 153.5° C. (onset temperature). In some embodiments, Compound I hemi-citrate Form I is further characterized by a DSC curve comprising an endotherm at about 82.3° C. (peak), an endotherm at about 140.5° C. (peak), and an endotherm at about 175.1° C. (peak). In some embodiments, Compound I hemi-citrate Form I is further characterized by a DSC curve as substantially shown in FIG. 54.

In some embodiments, Compound I hemi-citrate Form I is further characterized by TGA showing a weight loss of about 2.8 wt % from ambient temperature to about 100° C. In some embodiments, Compound I hemi-citrate Form I is further characterized by TGA comprising a thermogram substantially as shown in FIG. 55.

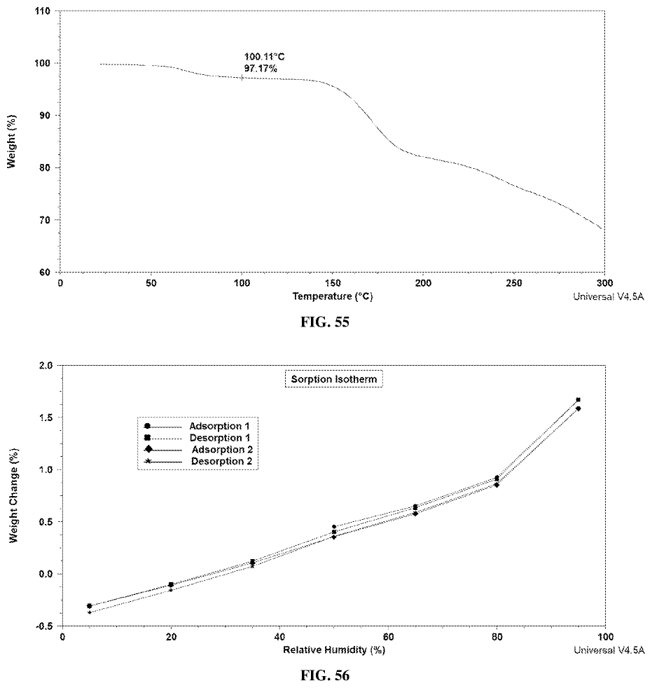

In some embodiments, Compound I hemi-citrate Form I is further characterized by a DVS curve substantially as shown in FIG. 56.

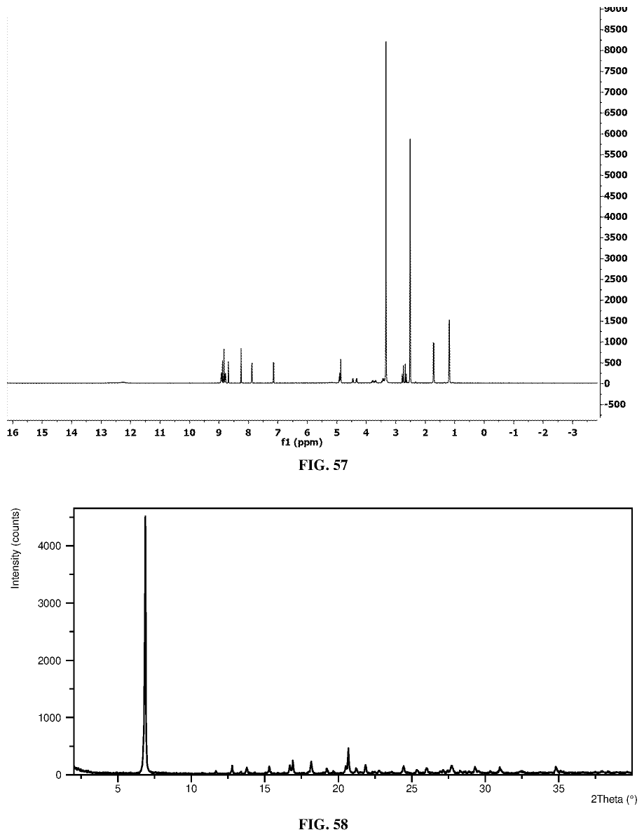

In some embodiments, Compound I hemi-citrate Form I is further characterized by a H NMR spectrum substantially as shown in FIG. 57.

In some embodiments, Compound I hemi-citrate Form I is a monohydrate. In some embodiments, Compound I hemi-citrate Form I comprises about 1 mole equivalent of water.

### Compound I Mono-HCl Form I

In one embodiment, provided is crystalline 4-(((R)-1-cyanoethyl)amino)-6-(3-cyanopyrrolo[1,2-b]pyridazin-7-yl)-N—((R)-2-fluoro-3-hydroxy-3-methylbutyl)nicotinamide mono-hydrochloride salt Form I (Compound I mono-HCl Form I), characterized by an X-ray powder diffractogram comprising peaks (±0.2°) at 6.9, 20.7, and 16.9°2θ as determined on a diffractometer using Cu-Kα radiation.

In some embodiments, Compound I mono-HCl Form I is further characterized by an X-ray powder diffractogram comprising one or more additional peaks (±0.2°) at 18.2, 12.8, or 21.8°2θ as determined on a diffractometer using Cu-Kα radiation. In some embodiments, Compound I mono-HCl Form I is further characterized by an X-ray powder diffractogram comprising one or more additional peaks (±0.2°) at 24.4, 15.3, or 21.2°2θ as determined on a diffractometer using Cu-Kα radiation. In some embodiments, Compound I mono-HCl Form I is further characterized by an X-ray powder diffractogram as substantially shown in FIG. 58.

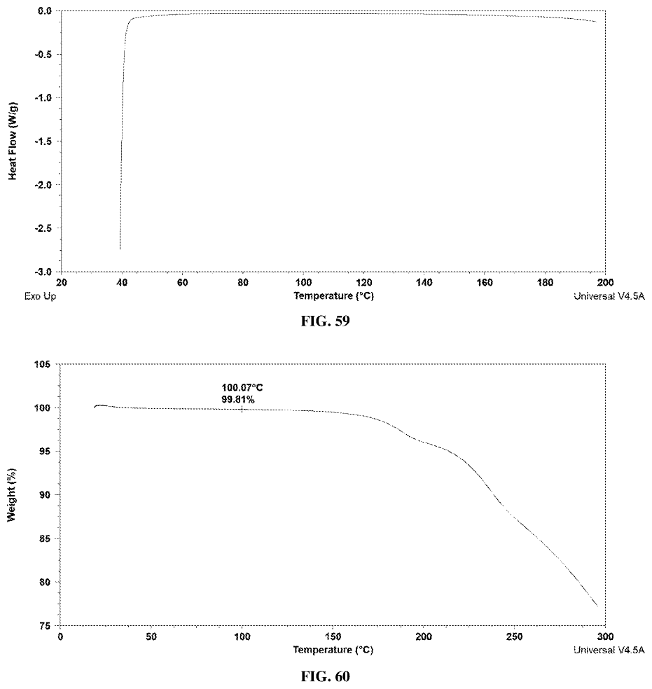

In some embodiments, Compound I mono-HCl Form I is further characterized by a DSC curve as substantially shown in FIG. 59.

In some embodiments, Compound I mono-HCl Form I is further characterized by TGA showing a weight loss of about 0.2 wt % from ambient temperature to about 100° C. In some embodiments, Compound I mono-HCl Form I is further characterized by TGA comprising a thermogram substantially as shown in FIG. 60.

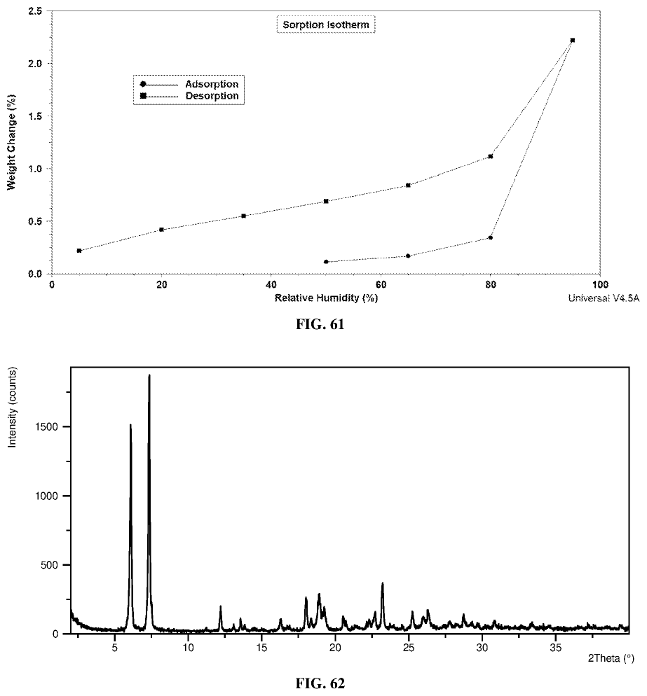

In some embodiments, Compound I mono-HCl Form I is further characterized by a DVS curve substantially as shown in FIG. 61.

In some embodiments, Compound I mono-HCl Form I is unsolvated.

### Compound I Mono-Maleate Form I

In one embodiment, provided is crystalline 4-(((R)-1-cyanoethyl)amino)-6-(3-cyanopyrrolo[1,2-b]pyridazin-7-yl)-N—((R)-2-fluoro-3-hydroxy-3-methylbutyl)nicotinamide mono-maleate salt Form I (Compound I mono-maleate Form I), characterized by an X-ray powder diffractogram comprising peaks (±0.2°) at 7.4, 6.1, and 23.2°2θ as determined on a diffractometer using Cu-Kα radiation.

In some embodiments, Compound I mono-maleate Form I is further characterized by an X-ray powder diffractogram comprising one or more additional peaks (±0.2°) at 18.0, 19.0, or 12.2°2θ as determined on a diffractometer using Cu-Kα radiation. In some embodiments, Compound I mono-maleate Form I is further characterized by an X-ray powder diffractogram comprising one or more additional peaks (±0.2°) at 25.2, 22.7, or 20.6°2θ as determined on a diffractometer using Cu-Kα radiation. In some embodiments, Compound I mono-maleate Form I is further characterized by an X-ray powder diffractogram as substantially shown in FIG. 62.

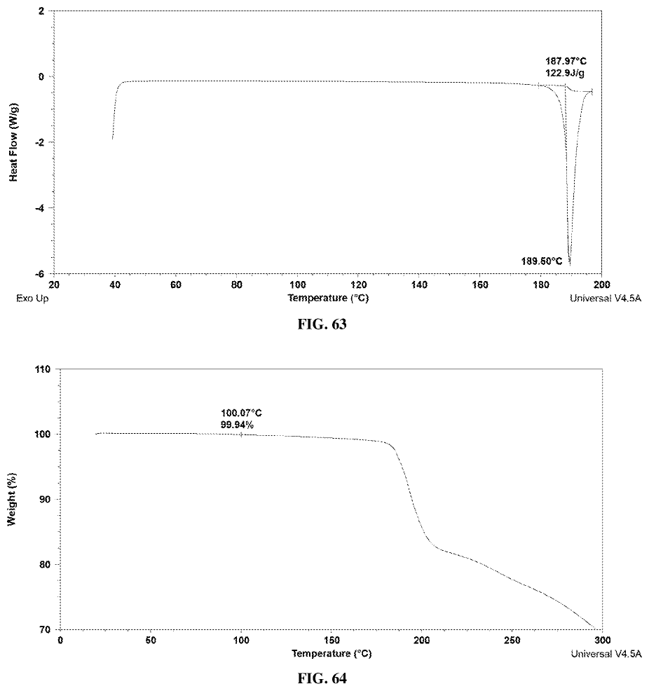

In some embodiments, Compound I mono-maleate Form I is further characterized by a DSC curve comprising an endotherm at about 188.0° C. (onset temperature). In some embodiments, Compound I mono-maleate Form I is further characterized by a DSC curve comprising an endotherm at about 189.5° C. (peak). In some embodiments, Compound I mono-maleate Form I is further characterized by a DSC curve as substantially shown in FIG. 63.

In some embodiments, Compound I mono-maleate Form I is further characterized by TGA showing a weight loss of about 0.1 wt % from ambient temperature to about 100° C. In some embodiments, Compound I mono-maleate Form I is further characterized by TGA comprising a thermogram substantially as shown in FIG. 64.

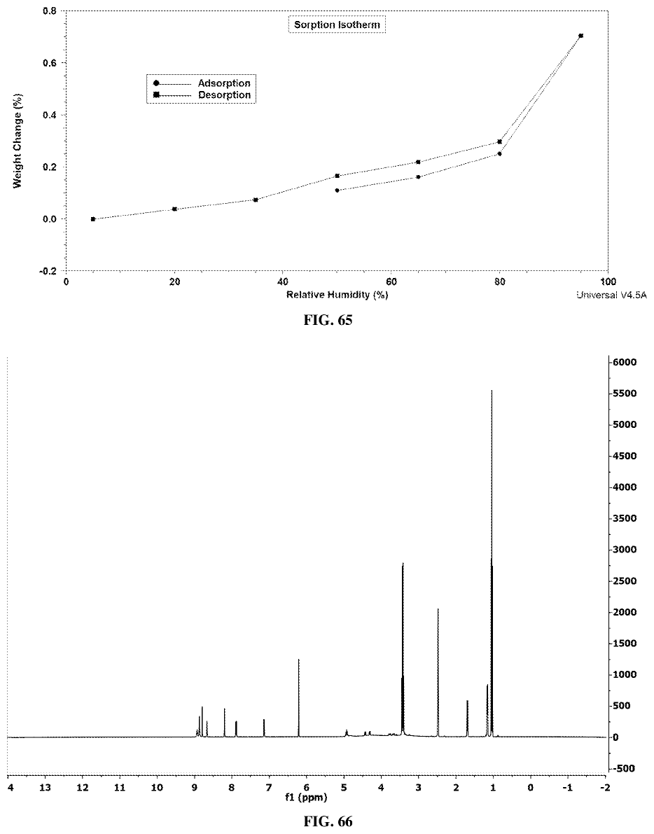

In some embodiments, Compound I mono-maleate Form I is further characterized by a DVS curve substantially as shown in FIG. 65.

In some embodiments, Compound I mono-maleate Form I is further characterized by a H NMR spectrum substantially as shown in FIG. 66.

In some embodiments, Compound I mono-maleate Form I is a unsolvated. In some embodiments, Compound I mono-maleate Form I comprises about 1 mole equivalent of maleic acid.

### Compound I Hemi-Fumarate Form I

In one embodiment, provided is crystalline 4-(((R)-1-cyanoethyl)amino)-6-(3-cyanopyrrolo[1,2-b]pyridazin-7-yl)-N—((R)-2-fluoro-3-hydroxy-3-methylbutyl)nicotinamide hemi-fumarate salt Form I (Compound I hemi-fumarate Form I), characterized by an X-ray powder diffractogram comprising peaks (±0.2°) at 7.7, 6.8, and 13.0°2θ as determined on a diffractometer using Cu-Kα radiation.

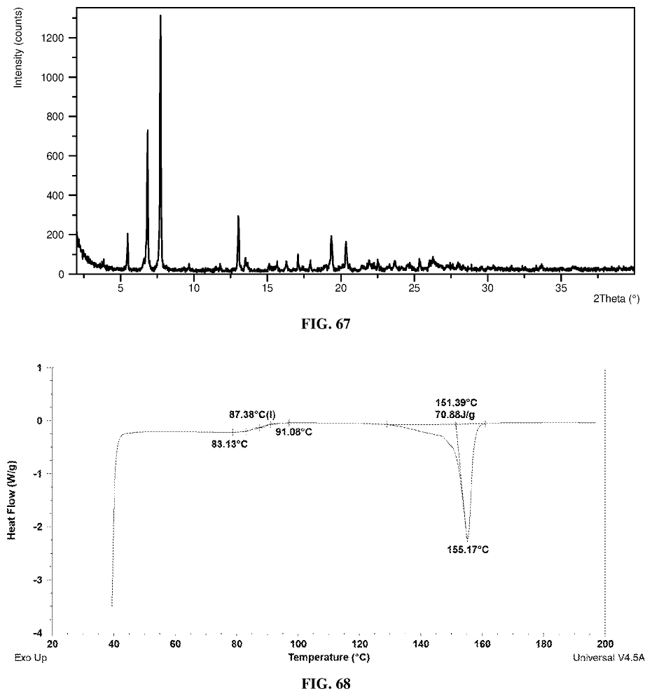

In some embodiments, Compound I hemi-fumarate Form I is further characterized by an X-ray powder diffractogram comprising one or more additional peaks (±0.2°) at 5.5, 19.4, or 20.3°2θ as determined on a diffractometer using Cu-Kα radiation. In some embodiments, Compound I hemi-fumarate Form I is further characterized by an X-ray powder diffractogram comprising one or more additional peaks (±0.2°) at 17.1, 13.5, or 23.7°2θ as determined on a diffractometer using Cu-Kα radiation. In some embodiments, Compound I hemi-fumarate Form I is further characterized by an X-ray powder diffractogram as substantially shown in FIG. 67.

In some embodiments, Compound I hemi-fumarate Form I is further characterized by a DSC curve comprising an endotherm at about 151.4° C. (onset temperature). In some embodiments, Compound I hemi-fumarate Form I is further characterized by a DSC curve comprising an endotherm at about 155.2° C. (peak). In some embodiments, Compound I hemi-fumarate Form I is further characterized by a DSC curve as substantially shown in FIG. 68.

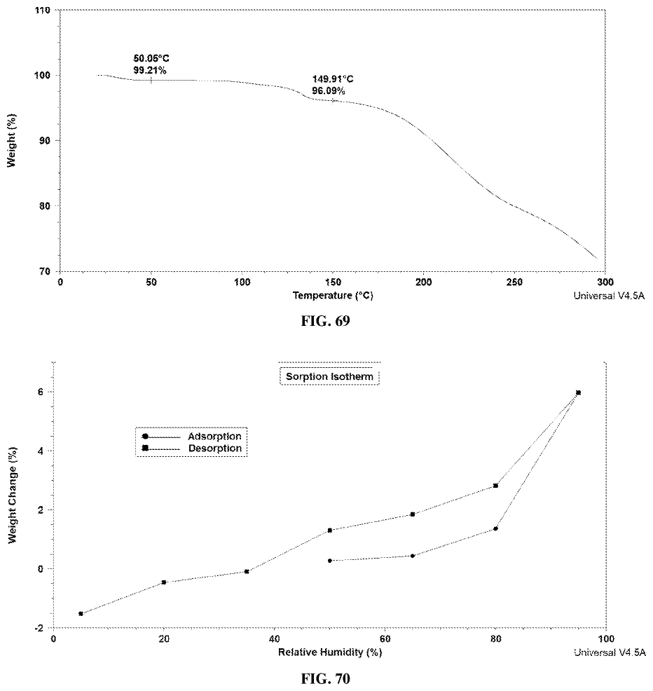

In some embodiments, Compound I hemi-fumarate Form I is further characterized by TGA showing a weight loss of about 0.8 wt % from ambient temperature to about 100° C. In some embodiments, Compound I hemi-fumarate Form I is further characterized by TGA comprising a thermogram substantially as shown in FIG. 69.

In some embodiments, Compound I hemi-fumarate Form I is further characterized by a DVS curve substantially as shown in FIG. 70.

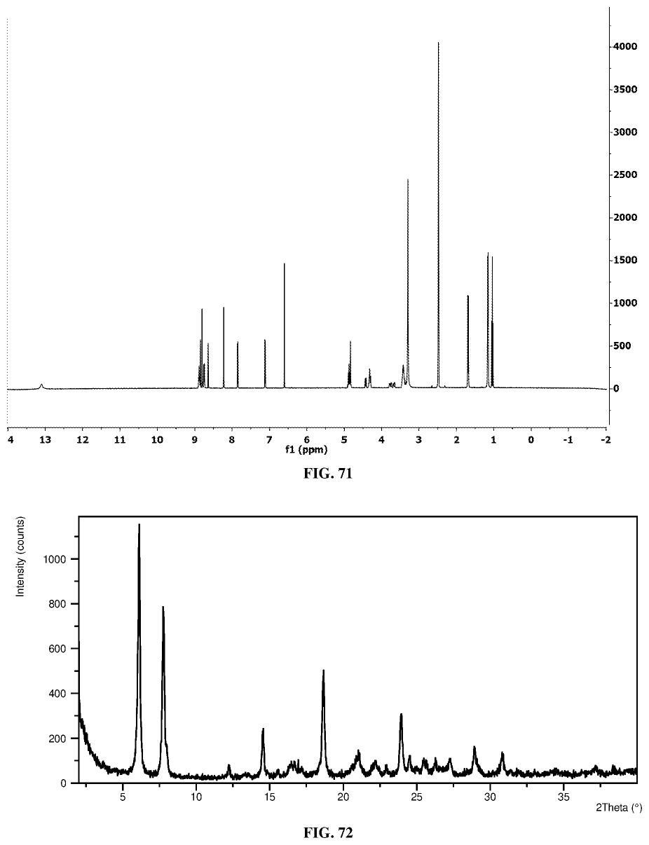

In some embodiments, Compound I hemi-fumarate Form I is further characterized by a H NMR spectrum substantially as shown in FIG. 71.

In some embodiments, Compound I hemi-fumarate Form I is a unsolvated. In some embodiments, Compound I hemi-fumarate Form I comprises about 0.5 mole equivalent of fumaric acid.

### Compound I Mono-Fumarate Form I

In one embodiment, provided is crystalline 4-(((R)-1-cyanoethyl)amino)-6-(3-cyanopyrrolo[1,2-b]pyridazin-7-yl)-N—((R)-2-fluoro-3-hydroxy-3-methylbutyl)nicotinamide mono-fumarate salt Form I (Compound I mono-fumarate Form I), characterized by an X-ray powder diffractogram comprising peaks (±0.2°) at 6.1, 7.8, and 18.7°2θ as determined on a diffractometer using Cu-Kα radiation.

In some embodiments, Compound I mono-fumarate Form I is further characterized by an X-ray powder diffractogram comprising one or more additional peaks (±0.2°) at 23.9, 14.5, or 21.0°2θ as determined on a diffractometer using Cu-Kα radiation. In some embodiments, Compound I mono-fumarate Form I is further characterized by an X-ray powder diffractogram comprising one or more additional peaks (±0.2°) at 24.5, 22.2, or 16.5°2θ as determined on a diffractometer using Cu-Kα radiation. In some embodiments, Compound I mono-fumarate Form I is further characterized by an X-ray powder diffractogram as substantially shown in FIG. 72.

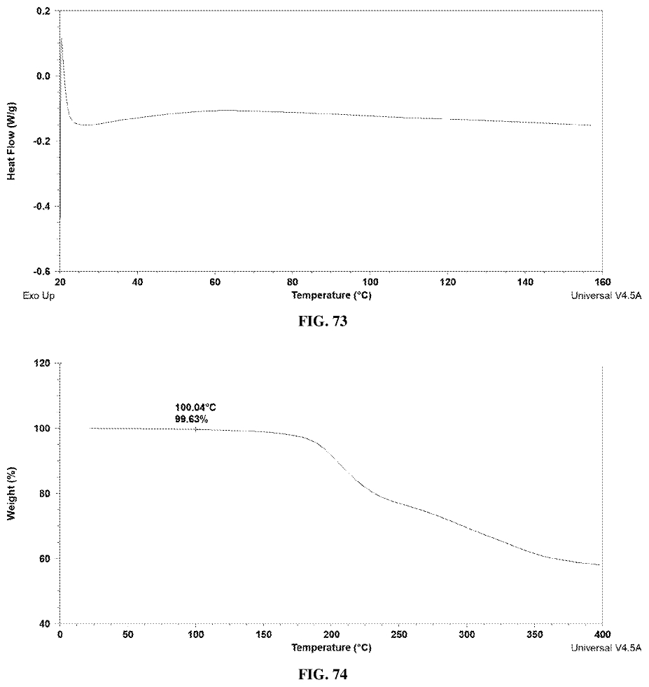

In some embodiments, Compound I mono-fumarate Form I is further characterized by a DSC curve as substantially shown in FIG. 73.

In some embodiments, Compound I mono-fumarate Form I is further characterized by TGA showing a weight loss of about 0.4 wt % from ambient temperature to about 100° C. In some embodiments, Compound I mono-fumarate Form I is further characterized by TGA comprising a thermogram substantially as shown in FIG. 74.

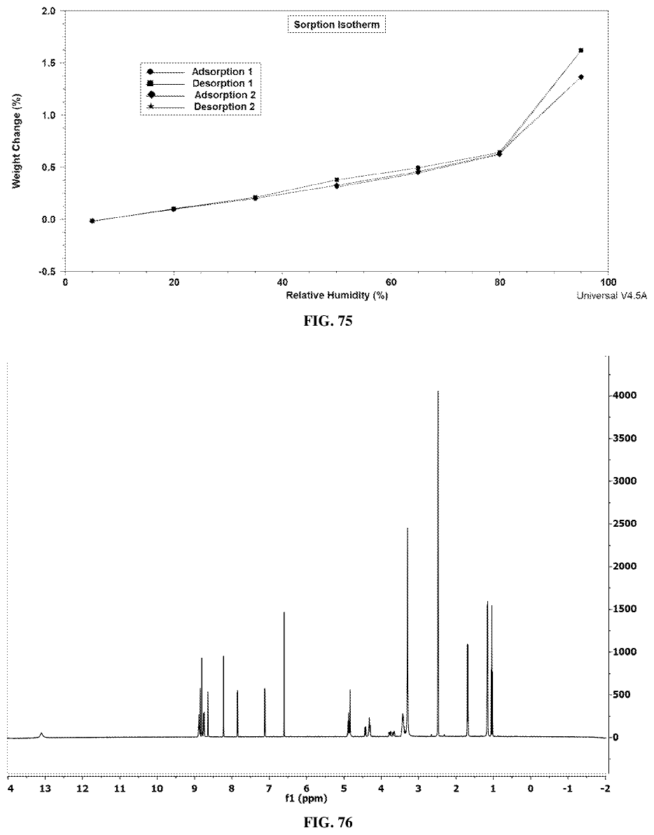

In some embodiments, Compound I mono-fumarate Form I is further characterized by a DVS curve substantially as shown in FIG. 75.

In some embodiments, Compound I mono-fumarate Form I is further characterized by a H NMR spectrum substantially as shown in FIG. 76.

In some embodiments, Compound I mono-fumarate Form I is a unsolvated. In some embodiments, Compound I mono-fumarate Form I comprises about 1.0 mole equivalent of fumaric acid.

### Compound I Hemi-L-Tartrate Form I

In one embodiment, provided is crystalline 4-(((R)-1-cyanoethyl)amino)-6-(3-cyanopyrrolo[1,2-b]pyridazin-7-yl)-N—((R)-2-fluoro-3-hydroxy-3-methylbutyl)nicotinamide hemi-L-tartrate salt Form I (Compound I hemi-L-tartrate Form I), characterized by an X-ray powder diffractogram comprising peaks (±0.2°) at 8.5, 5.2, and 18.6°2θ as determined on a diffractometer using Cu-Kα radiation.

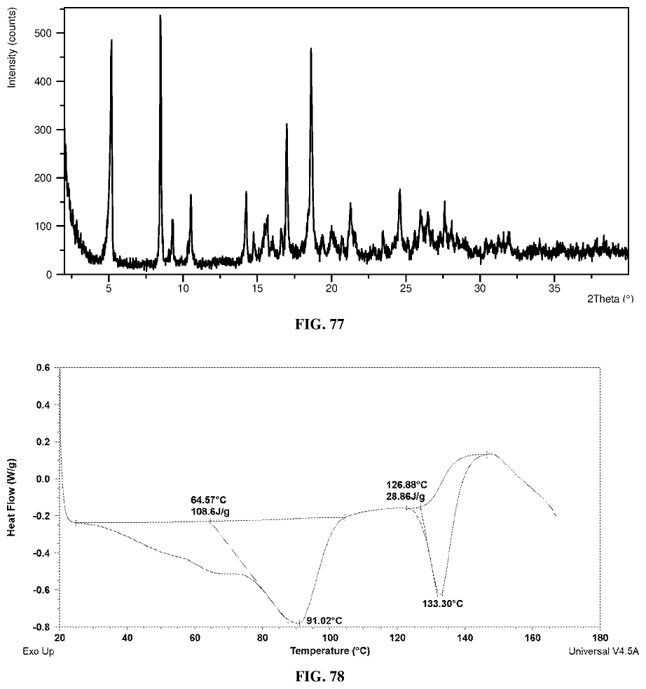

In some embodiments, Compound I hemi-L-tartrate Form I is further characterized by an X-ray powder diffractogram comprising one or more additional peaks (±0.2°) at 17.0, 10.5, or 14.3°2θ as determined on a diffractometer using Cu-Kα radiation. In some embodiments, Compound I hemi-L-tartrate Form I is further characterized by an X-ray powder diffractogram comprising one or more additional peaks (±0.2°) at 24.6, 21.3, or 9.3°2θ as determined on a diffractometer using Cu-Kα radiation. In some embodiments, Compound I hemi-L-tartrate Form I is further characterized by an X-ray powder diffractogram as substantially shown in FIG. 77.

In some embodiments, Compound I hemi-L-tartrate Form I is further characterized by a DSC curve comprising an endotherm at about 64.6° C. (onset temperature) and an endotherm at about 126.9° C. (onset temperature). In some embodiments, Compound I hemi-L-tartrate Form I is further characterized by a DSC curve comprising an endotherm at about 91.0° C. (peak) and an endotherm at about 133.3° C. (peak). In some embodiments, Compound I hemi-L-tartrate Form I is further characterized by a DSC curve as substantially shown in FIG. 78.

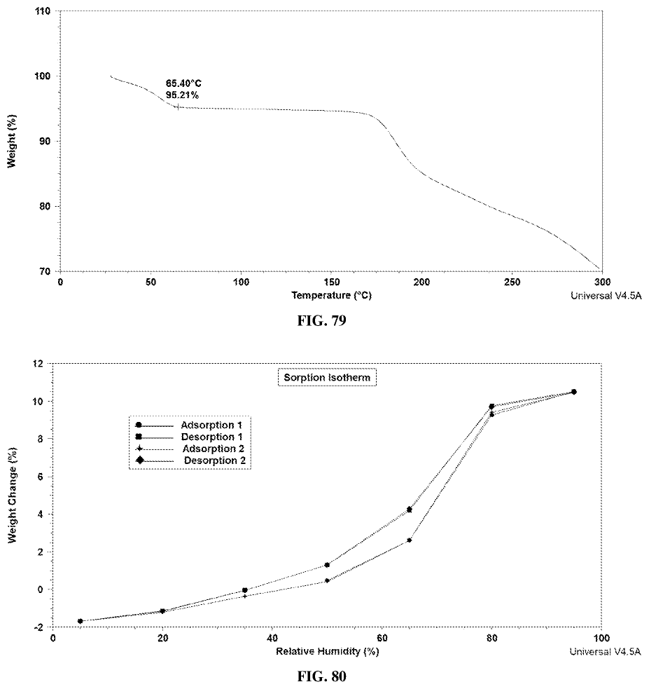

In some embodiments, Compound I hemi-L-tartrate Form I is further characterized by TGA showing a weight loss of about 4.8 wt % from ambient temperature to about 65° C. In some embodiments, Compound I hemi-L-tartrate Form I is further characterized by TGA comprising a thermogram substantially as shown in FIG. 79.

In some embodiments, Compound I hemi-L-tartrate Form I is further characterized by a DVS curve substantially as shown in FIG. 80.

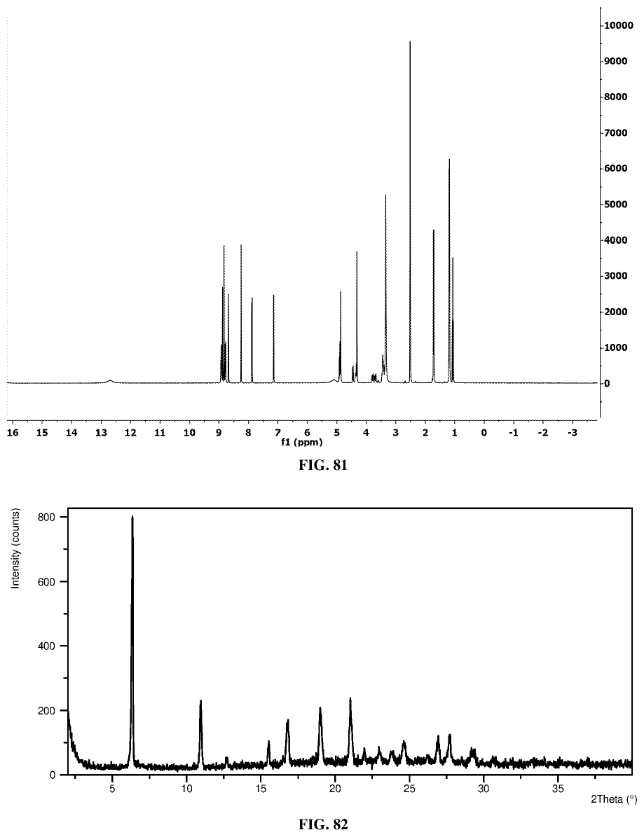

In some embodiments, Compound I hemi-L-tartrate Form I is further characterized by a H NMR spectrum substantially as shown in FIG. 81.

In some embodiments, Compound I hemi-L-tartrate Form I is a hydrate. In some embodiments, Compound I hemi-L-tartrate Form I comprises about 0.5 mole equivalent of L-tartaric acid.

### Compound I Mono-ESA Form I

In one embodiment, provided is crystalline 4-(((R)-1-cyanoethyl)amino)-6-(3-cyanopyrrolo[1,2-b]pyridazin-7-yl)-N—((R)-2-fluoro-3-hydroxy-3-methylbutyl)nicotinamide mono-ethanesulfonic (ESA) acid salt Form I (Compound I mono-ESA Form I), characterized by an X-ray powder diffractogram comprising peaks (±0.2°) at 6.4, 10.9, and 19.0°2θ as determined on a diffractometer using Cu-Kα radiation.

In some embodiments, Compound I mono-ESA Form I is further characterized by an X-ray powder diffractogram comprising one or more additional peaks (±0.2°) at 21.0, 16.8, or 15.6°2θ as determined on a diffractometer using Cu-Kα radiation. In some embodiments, Compound I mono-ESA Form I is further characterized by an X-ray powder diffractogram comprising one or more additional peaks (±0.2°) at 24.6, 22.0, or 23.0°2θ as determined on a diffractometer using Cu-Kα radiation. In some embodiments, Compound I mono-ESA Form I is further characterized by an X-ray powder diffractogram as substantially shown in FIG. 82.

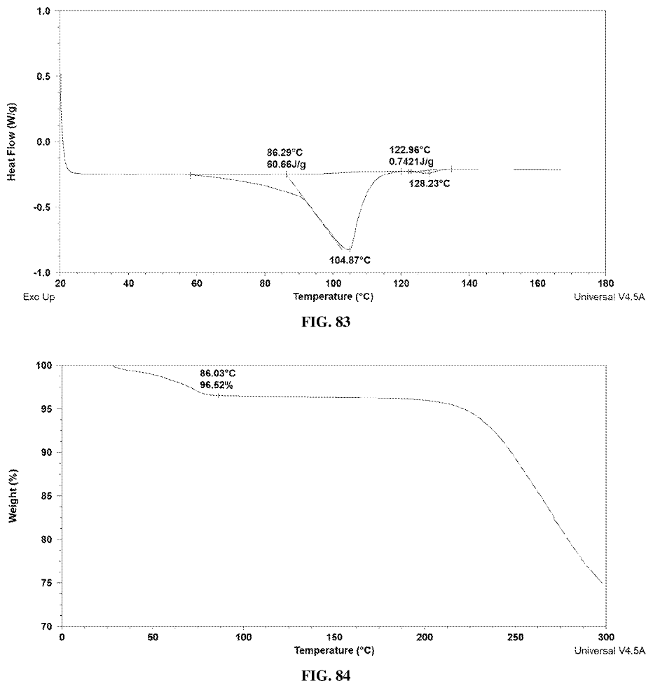

In some embodiments, Compound I mono-ESA Form I is further characterized by a DSC curve comprising an endotherm at about 86.3° C. (onset temperature) and an endotherm at about 123.0° C. (onset temperature). In some embodiments, Compound I mono-ESA Form I is further characterized by a DSC curve comprising an endotherm at about 104.9° C. (peak) and an endotherm at 128.2° C. In some embodiments, Compound I mono-ESA Form I is further characterized by a DSC curve as substantially shown in FIG. 83.

In some embodiments, Compound I mono-ESA Form I is further characterized by TGA showing a weight loss of about 3.5 wt % from ambient temperature to about 86° C. In some embodiments, Compound I mono-ESA Form I is further characterized by TGA comprising a thermogram substantially as shown in FIG. 84.

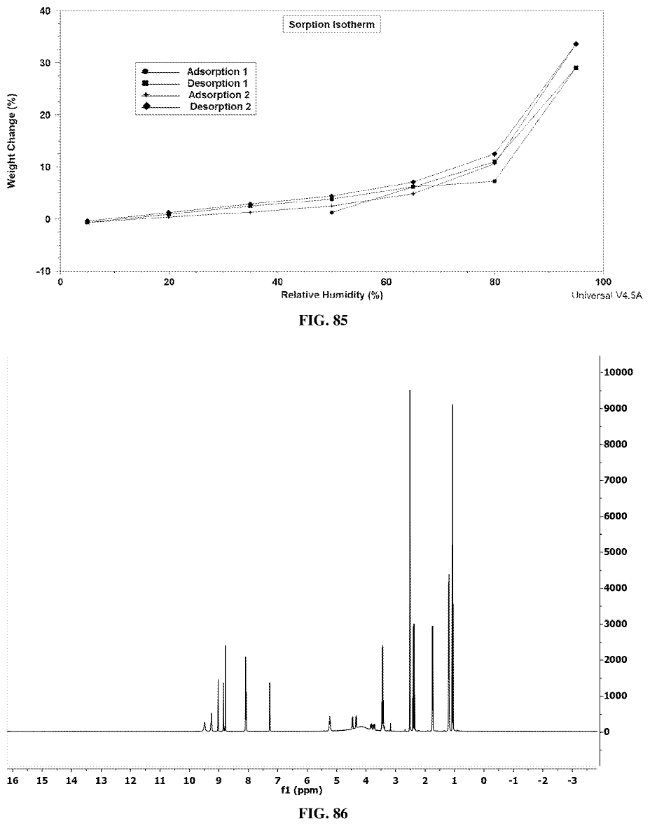

In some embodiments, Compound I mono-ESA Form I is further characterized by a DVS curve substantially as shown in FIG. 85.

In some embodiments, Compound I mono-ESA Form I is further characterized by a H NMR spectrum substantially as shown in FIG. 86.

In some embodiments, Compound I mono-ESA Form I is a hydrate. In some embodiments, Compound I mono-ESA Form I comprises about 1.0 mole equivalent of ethylsulfonic acid.

### Compound I Hemi-Glycolate Form I

In one embodiment, provided is crystalline 4-(((R)-1-cyanoethyl)amino)-6-(3-cyanopyrrolo[1,2-b]pyridazin-7-yl)-N—((R)-2-fluoro-3-hydroxy-3-methylbutyl)nicotinamide hemi-glycolate salt Form I (Compound I hemi-glycolate Form I), characterized by an X-ray powder diffractogram comprising peaks (±0.2°) at 6.2, 8.0, and 22.9°2θ as determined on a diffractometer using Cu-Kα radiation.

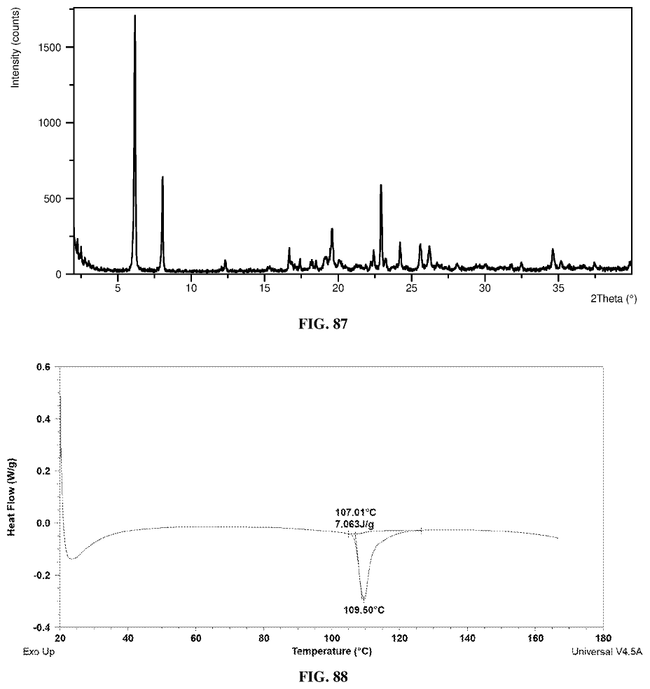

In some embodiments, Compound I hemi-glycolate Form I is further characterized by an X-ray powder diffractogram comprising one or more additional peaks (±0.2°) at 19.6, 2.3, or 25.6°2θ as determined on a diffractometer using Cu-Kα radiation. In some embodiments, Compound I hemi-glycolate Form I is further characterized by an X-ray powder diffractogram comprising one or more additional peaks (±0.2°) at 24.2, 22.4, or 16.7°2θ as determined on a diffractometer using Cu-Kα radiation. In some embodiments, Compound I hemi-glycolate Form I is further characterized by an X-ray powder diffractogram as substantially shown in FIG. 87.

In some embodiments, Compound I hemi-glycolate Form I is further characterized by a DSC curve comprising an endotherm at about 107.0° C. (onset temperature). In some embodiments, Compound I hemi-glycolate Form I is further characterized by a DSC curve comprising an endotherm at about 109.5° C. (peak). In some embodiments, Compound I hemi-glycolate Form I is further characterized by a DSC curve as substantially shown in FIG. 88.

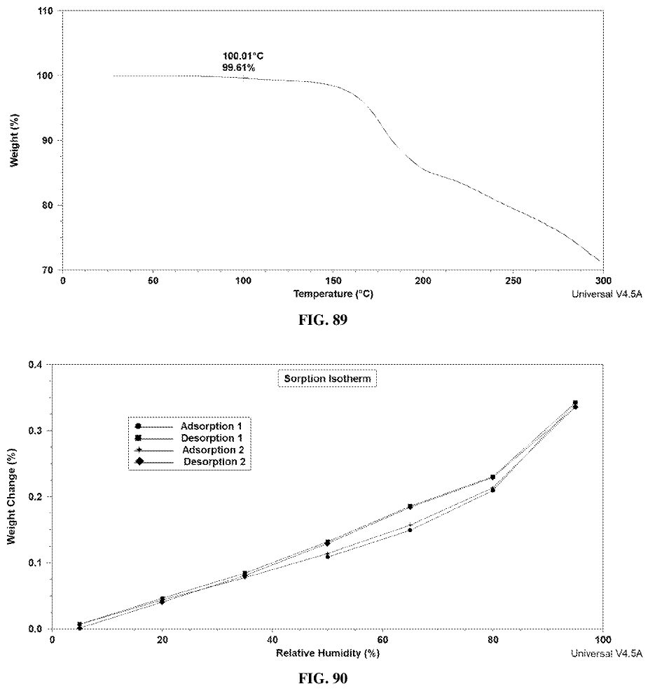

In some embodiments, Compound I hemi-glycolate Form I is further characterized by TGA showing a weight loss of about 0.4 wt % from ambient temperature to about 100° C. In some embodiments, Compound I hemi-glycolate Form I is further characterized by TGA comprising a thermogram substantially as shown in FIG. 89.

In some embodiments, Compound I hemi-glycolate Form I is further characterized by a DVS curve substantially as shown in FIG. 90.

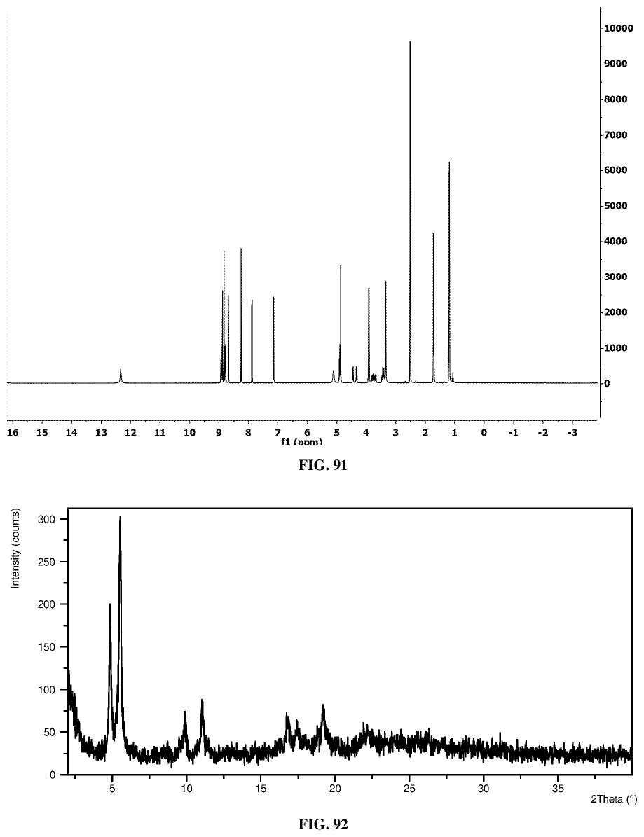

In some embodiments, Compound I hemi-glycolate Form I is further characterized by a H NMR spectrum substantially as shown in FIG. 91.

In some embodiments, Compound I hemi-glycolate Form I is unsolvated. In some embodiments, Compound I hemi-glycolate Form I comprises about 0.5 mole equivalent of glycolic acid.

### Compound I Sulfate Form I

In one embodiment, provided is crystalline 4-(((R)-1-cyanoethyl)amino)-6-(3-cyanopyrrolo[1,2-b]pyridazin-7-yl)-N—((R)-2-fluoro-3-hydroxy-3-methylbutyl)nicotinamide sulfate salt Form I (Compound I sulfate Form I), characterized by an X-ray powder diffractogram comprising peaks (±0.2°) at 5.5, 4.9, and 11.1°2θ as determined on a diffractometer using Cu-Kα radiation.

In some embodiments, Compound I sulfate Form I is further characterized by an X-ray powder diffractogram comprising one or more additional peaks (±0.2°) at 19.2, 9.9, or 16.8°2θ as determined on a diffractometer using Cu-Kα radiation. In some embodiments, Compound I sulfate Form I is further characterized by an X-ray powder diffractogram comprising one or more additional peaks (±0.2°) at 17.4, 22.1, or 5.5°2θ as determined on a diffractometer using Cu-Kα radiation. In some embodiments, Compound I sulfate Form I is further characterized by an X-ray powder diffractogram as substantially shown in FIG. 92.

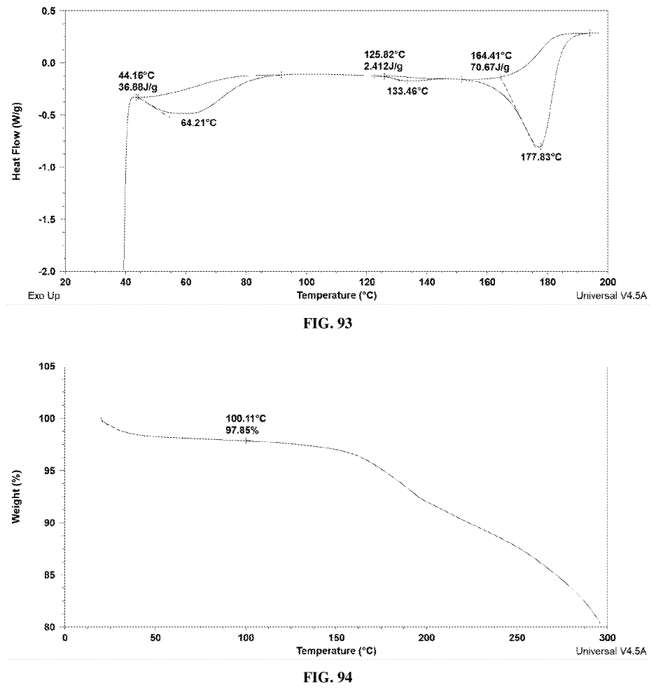

In some embodiments, Compound I sulfate Form I is further characterized by a DSC curve comprising an endotherm at about 44.2° C. (onset temperature), an endotherm at about 125.8° C. (onset temperature), and an endotherm at about 164.4° C. (onset temperature). In some embodiments, Compound I sulfate Form I is further characterized by a DSC curve comprising an endotherm at about 64.2° C. (peak), an endotherm at about 133.5° C. (peak), and an endotherm at about 177.8° C. (peak). In some embodiments, Compound I sulfate Form I is further characterized by a DSC curve as substantially shown in FIG. 93.

In some embodiments, Compound I sulfate Form I is further characterized by TGA showing a weight loss of about 2.1 wt % from ambient temperature to about 100° C. In some embodiments, Compound I sulfate Form I is further characterized by TGA comprising a thermogram substantially as shown in FIG. 94.

In some embodiments Compound I sulfate Form I is solvated. In some embodiments Compound I sulfate Form I is a hydrate. In some embodiments Compound I sulfate Form I is an ethanol solvate.

### Compound I Phosphate Form I

In one embodiment, provided is crystalline 4-(((R)-1-cyanoethyl)amino)-6-(3-cyanopyrrolo[1,2-b]pyridazin-7-yl)-N—((R)-2-fluoro-3-hydroxy-3-methylbutyl)nicotinamide phosphate salt Form I (Compound I phosphate Form I), characterized by an X-ray powder diffractogram comprising peaks (±0.2°) at 4.8, 9.7, and 16.9°2θ as determined on a diffractometer using Cu-Kα radiation.

In some embodiments, Compound I phosphate Form I is further characterized by an X-ray powder diffractogram comprising one or more additional peaks (±0.2°) at 17.9, 10.3, or 22.0°2θ as determined on a diffractometer using Cu-Kα radiation. In some embodiments, Compound I phosphate Form I is further characterized by an X-ray powder diffractogram comprising one or more additional peaks (±0.2°) at 19.2, 23.6, or 25.9°2θ as determined on a diffractometer using Cu-Kα radiation. In some embodiments, Compound I phosphate Form I is further characterized by an X-ray powder diffractogram as substantially shown in FIG. 95.

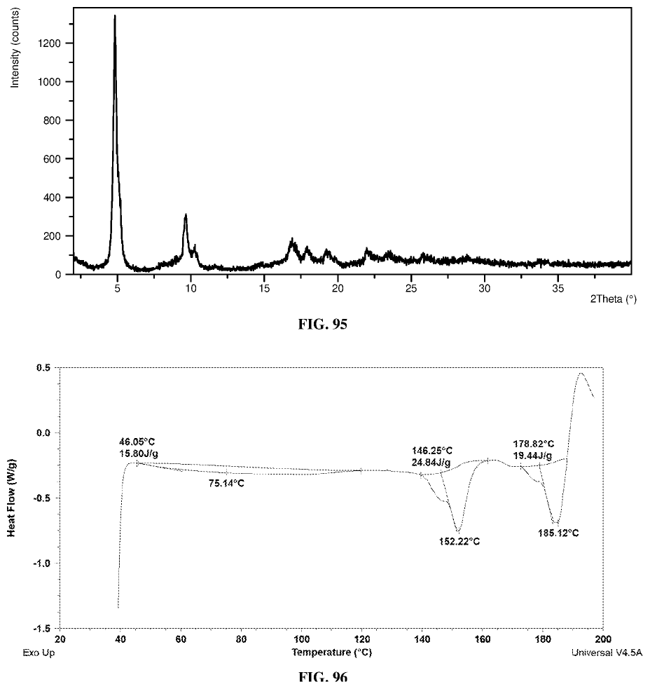

In some embodiments, Compound I phosphate Form I is further characterized by a DSC curve comprising an endotherm at about 46.1° C. (onset temperature), an endotherm at about 146.3° C. (onset temperature), and an endotherm at about 178.8° C. (onset temperature). In some embodiments, Compound I phosphate Form I is further characterized by a DSC curve comprising an endotherm at about 75.1° C. (peak), an endotherm at about 152.2° C. (peak), and an endotherm at about 185.1° C. (peak). In some embodiments, Compound I phosphate Form I is further characterized by a DSC curve as substantially shown in FIG. 96.

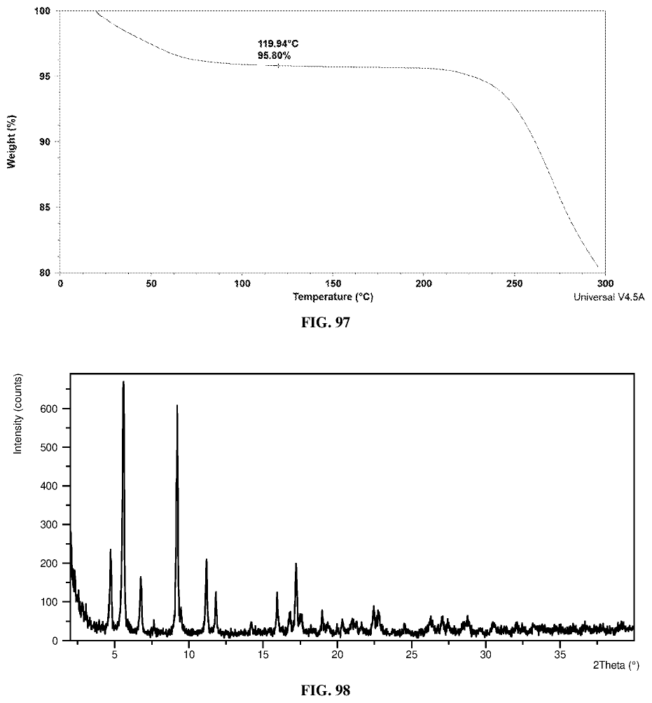

In some embodiments, Compound I phosphate Form I is further characterized by TGA showing a weight loss of about 4.2 wt % from ambient temperature to about 120° C. In some embodiments, Compound I phosphate Form I is further characterized by TGA comprising a thermogram substantially as shown in FIG. 97.

In some embodiments Compound I phosphate Form I is unsolvated.

### Compound I Hemi-HCl Form I

In one embodiment, provided is crystalline 4-(((R)-1-cyanoethyl)amino)-6-(3-cyanopyrrolo[1,2-b]pyridazin-7-yl)-N—((R)-2-fluoro-3-hydroxy-3-methylbutyl)nicotinamide hemi-HCl salt Form I (Compound I hemi-HCl Form I), characterized by an X-ray powder diffractogram comprising peaks (±0.2°) at 5.6, 9.2, and 11.2°2θ as determined on a diffractometer using Cu-Kα radiation.

In some embodiments, Compound I hemi-HCl Form I is further characterized by an X-ray powder diffractogram comprising one or more additional peaks (±0.2°) at 4.7, 17.2, or 6.8°2θ as determined on a diffractometer using Cu-Kα radiation. In some embodiments, Compound I hemi-HCl Form I is further characterized by an X-ray powder diffractogram comprising one or more additional peaks (±0.2°) at 11.8, 15.9, or 19.0°2θ as determined on a diffractometer using Cu-Kα radiation. In some embodiments, Compound I hemi-HCl Form I is further characterized by an X-ray powder diffractogram as substantially shown in FIG. 98.

In some embodiments, Compound I hemi-HCl Form I is further characterized by a DSC curve comprising an endotherm at about 79.9° C. (onset temperature), and an endotherm at about 136.7° C. (onset temperature). In some embodiments, Compound I hemi-HCl Form I is further characterized by a DSC curve comprising an endotherm at about 112.3° C. (peak) and an endotherm at about 140.4° C. (peak). In some embodiments, Compound I hemi-HCl Form I is further characterized by a DSC curve as substantially shown in FIG. 99.

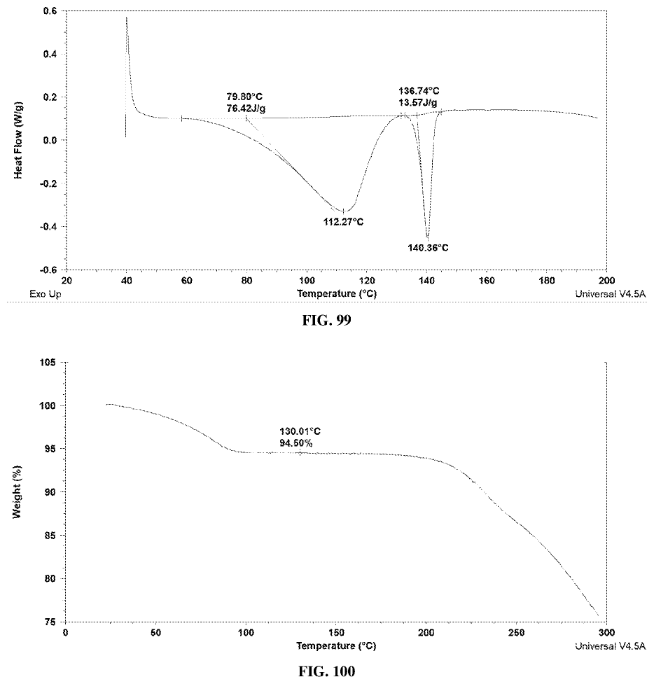

In some embodiments, Compound I hemi-HCl Form I is further characterized by TGA showing a weight loss of about 5.5 wt % from ambient temperature to about 130° C. In some embodiments, Compound I hemi-HCl Form I is further characterized by TGA comprising a thermogram substantially as shown in FIG. 100.

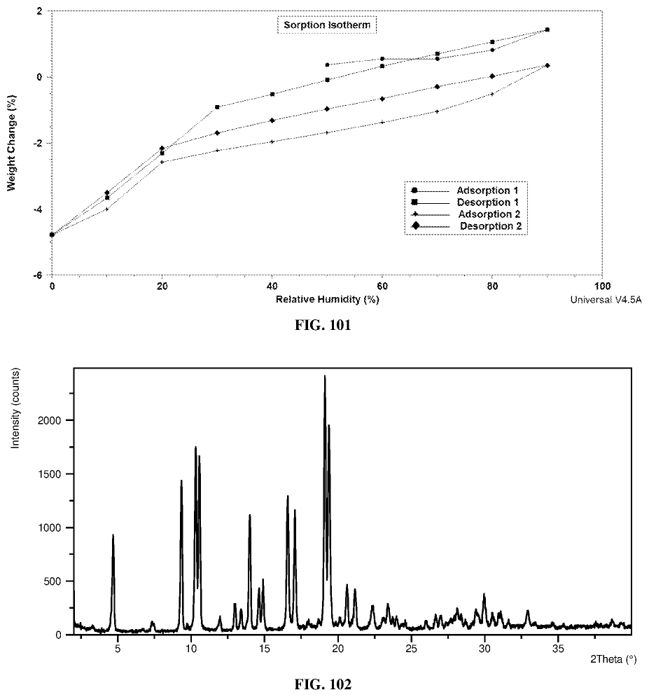

In some embodiments, Compound I hemi-HCl Form I is further characterized by a DVS curve substantially as shown in FIG. 101.

In some embodiments Compound I hemi-HCl Form I is a hydrate.

### Compound I Co-Crystal Form I

In one embodiment, provided is a crystalline co-crystal of Compound I and 4-(((S)-1-cyanoethyl)amino)-6-(3-cyanopyrrolo[1,2-b]pyridazin-7-yl)-N—((R)-2-fluoro-3-hydroxy-3-methylbutyl)nicotinamide (Compound I co-crystal Form I), characterized by an X-ray powder diffractogram comprising peaks (±0.2°) at 19.1, 10.3, and 9.4°2θ as determined on a diffractometer using Cu-Kα radiation.

In some embodiments, Compound I co-crystal Form I is further characterized by an X-ray powder diffractogram comprising one or more additional peaks (±0.2°) at 16.6, 17.1, and 14.0°2θ as determined on a diffractometer using Cu-Kα radiation. In some embodiments, Compound I co-crystal Form I is further characterized by an X-ray powder diffractogram comprising one or more additional peaks (±0.2°) at 4.7, 14.9, and 20.6°2θ as determined on a diffractometer using Cu-Kα radiation. In some embodiments, Compound I co-crystal Form I is further characterized by an X-ray powder diffractogram as substantially shown in FIG. 102.

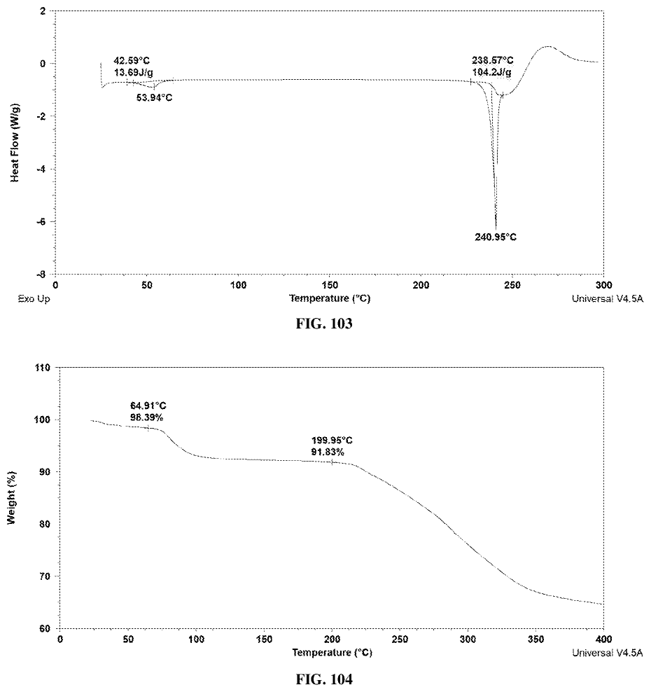

In some embodiments, Compound I co-crystal Form I is further characterized by a DSC curve comprising an endotherm at about 42.6° C. (onset temperature) and an endotherm at about 238.6° C. (onset temperature). In some embodiments, Compound I co-crystal Form I is further characterized by a DSC curve comprising an endotherm at about 53.9° C. (peak) and an endotherm at about 241.0° C. (peak). In some embodiments, Compound I co-crystal Form I is further characterized by a DSC curve as substantially shown in FIG. 103.

In some embodiments, Compound I co-crystal Form I is further characterized by TGA showing a weight loss of about 1.6 wt % from ambient temperature to about 65° C. and a weight loss of about 6.6 wt % from about 65° C. to about 200° C. In some embodiments, Compound I co-crystal Form I is further characterized by TGA comprising a thermogram substantially as shown in FIG. 104.

In some embodiments Compound I co-crystal Form I is solvated. In some embodiments Compound I co-crystal Form I is a 2-MeTHF solvate.

### Compositions

In some embodiments, provided is a composition comprising a salt or solid form of 4-(((R)-1-cyanoethyl)amino)-6-(3-cyanopyrrolo[1,2-b]pyridazin-7-yl)-N—((R)-2-fluoro-3-hydroxy-3-methylbutyl)nicotinamide (Compound I), as described herein.

In one embodiment, provided is a composition comprising a salt or solid form of 4-(((R)-1-cyanoethyl)amino)-6-(3-cyanopyrrolo[1,2-b]pyridazin-7-yl)-N—((R)-2-fluoro-3-hydroxy-3-methylbutyl)nicotinamide (Compound I), or salt or solvate thereof, wherein at least 50% to 99% (e.g., at least 50%, at least 55%, at least 60%, at least 65%, at least 70%, at least 75%, at least 80%, at least 85%, at least 90%, at least 95%, or at least 99%) of Compound I present in a composition is in the designated salt, solid form, crystalline form, or crystalline salt form.

In one embodiment, provided is a composition comprising 4-(((R)-1-cyanoethyl)amino)-6-(3-cyanopyrrolo[1,2-b]pyridazin-7-yl)-N—((R)-2-fluoro-3-hydroxy-3-methylbutyl)nicotinamide Form I (Compound I Form I), or solvate thereof, wherein at least 50% to 99% (e.g., at least 50%, at least 55%, at least 60%, at least 65%, at least 70%, at least 75%, at least 80%, at least 85%, at least 90%, at least 95%, or at least 99%) of Compound I present in the composition is Compound I Form I.

In one embodiment, provided is a composition comprising 4-(((R)-1-cyanoethyl)amino)-6-(3-cyanopyrrolo[1,2-b]pyridazin-7-yl)-N—((R)-2-fluoro-3-hydroxy-3-methylbutyl)nicotinamide Form II (Compound I Form II), or solvate thereof, wherein at least 50% to 99% (e.g., at least 50%, at least 55%, at least 60%, at least 65%, at least 70%, at least 75%, at least 80%, at least 85%, at least 90%, at least 95%, or at least 99%) of Compound I present in the composition is Compound I Form II.

In one embodiment, provided is a composition comprising 4-(((R)-1-cyanoethyl)amino)-6-(3-cyanopyrrolo[1,2-b]pyridazin-7-yl)-N—((R)-2-fluoro-3-hydroxy-3-methylbutyl)nicotinamide Form III (Compound I Form III), or solvate thereof, wherein at least 50% to 99% (e.g., at least 50%, at least 55%, at least 60%, at least 65%, at least 70%, at least 75%, at least 80%, at least 85%, at least 90%, at least 95%, or at least 99%) of Compound I present in the composition is Compound I Form III.

In one embodiment, provided is a composition comprising 4-(((R)-1-cyanoethyl)amino)-6-(3-cyanopyrrolo[1,2-b]pyridazin-7-yl)-N—((R)-2-fluoro-3-hydroxy-3-methylbutyl)nicotinamide Form IV (Compound I Form IV), or solvate thereof, wherein at least 50% to 99% (e.g., at least 50%, at least 55%, at least 60%, at least 65%, at least 70%, at least 75%, at least 80%, at least 85%, at least 90%, at least 95%, or at least 99%) of Compound I present in the composition is Compound I Form IV.

In one embodiment, provided is a composition comprising 4-(((R)-1-cyanoethyl)amino)-6-(3-cyanopyrrolo[1,2-b]pyridazin-7-yl)-N—((R)-2-fluoro-3-hydroxy-3-methylbutyl)nicotinamide Form V (Compound I Form V), or solvate thereof, wherein at least 50% to 99% (e.g., at least 50%, at least 55%, at least 60%, at least 65%, at least 70%, at least 75%, at least 80%, at least 85%, at least 90%, at least 95%, or at least 99%) of Compound I present in the composition is Compound I Form V.

In one embodiment, provided is a composition comprising 4-(((R)-1-cyanoethyl)amino)-6-(3-cyanopyrrolo[1,2-b]pyridazin-7-yl)-N—((R)-2-fluoro-3-hydroxy-3-methylbutyl)nicotinamide Form VI (Compound I Form VI), or solvate thereof, wherein at least 50% to 99% (e.g., at least 50%, at least 55%, at least 60%, at least 65%, at least 70%, at least 75%, at least 80%, at least 85%, at least 90%, at least 95%, or at least 99%) of Compound I present in the composition is Compound I Form VI.

In one embodiment, provided is a composition comprising 4-(((R)-1-cyanoethyl)amino)-6-(3-cyanopyrrolo[1,2-b]pyridazin-7-yl)-N—((R)-2-fluoro-3-hydroxy-3-methylbutyl)nicotinamide Form VII (Compound I Form VII), or solvate thereof, wherein at least 50% to 99% (e.g., at least 50%, at least 55%, at least 60%, at least 65%, at least 70%, at least 75%, at least 80%, at least 85%, at least 90%, at least 95%, or at least 99%) of Compound I present in the composition is Compound I Form VII.

In one embodiment, provided is a composition comprising 4-(((R)-1-cyanoethyl)amino)-6-(3-cyanopyrrolo[1,2-b]pyridazin-7-yl)-N—((R)-2-fluoro-3-hydroxy-3-methylbutyl)nicotinamide Form VIII (Compound I Form VIII), or solvate thereof, wherein at least 50% to 99% (e.g., at least 50%, at least 55%, at least 60%, at least 65%, at least 70%, at least 75%, at least 80%, at least 85%, at least 90%, at least 95%, or at least 99%) of Compound I present in the composition is Compound I Form VIII.

In one embodiment, provided is a composition comprising 4-(((R)-1-cyanoethyl)amino)-6-(3-cyanopyrrolo[1,2-b]pyridazin-7-yl)-N—((R)-2-fluoro-3-hydroxy-3-methylbutyl)nicotinamide Form IX (Compound I Form IX), or solvate thereof, wherein at least 50% to 99% (e.g., at least 50%, at least 55%, at least 60%, at least 65%, at least 70%, at least 75%, at least 80%, at least 85%, at least 90%, at least 95%, or at least 99%) of Compound I present in the composition is Compound I Form IX.

In one embodiment, provided is a composition comprising 4-(((R)-1-cyanoethyl)amino)-6-(3-cyanopyrrolo[1,2-b]pyridazin-7-yl)-N—((R)-2-fluoro-3-hydroxy-3-methylbutyl)nicotinamide Form X (Compound I Form X), or solvate thereof, wherein at least 50% to 99% (e.g., at least 50%, at least 55%, at least 60%, at least 65%, at least 70%, at least 75%, at least 80%, at least 85%, at least 90%, at least 95%, or at least 99%) of Compound I present in the composition is Compound I Form X.

In one embodiment, provided is a composition comprising 4-(((R)-1-cyanoethyl)amino)-6-(3-cyanopyrrolo[1,2-b]pyridazin-7-yl)-N—((R)-2-fluoro-3-hydroxy-3-methylbutyl)nicotinamide Form XI (Compound I Form XI), or solvate thereof, wherein at least 50% to 99% (e.g., at least 50%, at least 55%, at least 60%, at least 65%, at least 70%, at least 75%, at least 80%, at least 85%, at least 90%, at least 95%, or at least 99%) of Compound I present in the composition is Compound I Form XI.

In one embodiment, provided is a composition comprising 4-(((R)-1-cyanoethyl)amino)-6-(3-cyanopyrrolo[1,2-b]pyridazin-7-yl)-N—((R)-2-fluoro-3-hydroxy-3-methylbutyl)nicotinamide Form XII (Compound I Form XII), or solvate thereof, wherein at least 50% to 99% (e.g., at least 50%, at least 55%, at least 60%, at least 65%, at least 70%, at least 75%, at least 80%, at least 85%, at least 90%, at least 95%, or at least 99%) of Compound I present in the composition is Compound I Form XII.

In one embodiment, provided is a composition comprising 4-(((R)-1-cyanoethyl)amino)-6-(3-cyanopyrrolo[1,2-b]pyridazin-7-yl)-N—((R)-2-fluoro-3-hydroxy-3-methylbutyl)nicotinamide Form XIII (Compound I Form XIII), or solvate thereof, wherein at least 50% to 99% (e.g., at least 50%, at least 55%, at least 60%, at least 65%, at least 70%, at least 75%, at least 80%, at least 85%, at least 90%, at least 95%, or at least 99%) of Compound I present in the composition is Compound I Form XIII.

In one embodiment, provided is a composition comprising 4-(((R)-1-cyanoethyl)amino)-6-(3-cyanopyrrolo[1,2-b]pyridazin-7-yl)-N—((R)-2-fluoro-3-hydroxy-3-methylbutyl)nicotinamide amorphous Form I (Compound I amorphous Form), or solvate thereof, wherein at least 50% to 99% (e.g., at least 50%, at least 55%, at least 60%, at least 65%, at least 70%, at least 75%, at least 80%, at least 85%, at least 90%, at least 95%, or at least 99%) of Compound I present in the composition is Compound I amorphous Form.

In one embodiment, provided is a composition comprising 4-(((R)-1-cyanoethyl)amino)-6-(3-cyanopyrrolo[1,2-b]pyridazin-7-yl)-N—((R)-2-fluoro-3-hydroxy-3-methylbutyl)nicotinamide mono-citrate salt Form I (Compound I mono-citrate Form I), or solvate thereof, wherein at least 50% to 99% (e.g., at least 50%, at least 55%, at least 60%, at least 65%, at least 70%, at least 75%, at least 80%, at least 85%, at least 90%, at least 95%, or at least 99%) of Compound I present in the composition is Compound I mono-citrate Form I.

In one embodiment, provided is a composition comprising 4-(((R)-1-cyanoethyl)amino)-6-(3-cyanopyrrolo[1,2-b]pyridazin-7-yl)-N—((R)-2-fluoro-3-hydroxy-3-methylbutyl)nicotinamide mono-citrate salt Form II (Compound I mono-citrate Form II), or solvate thereof, wherein at least 50% to 99% (e.g., at least 50%, at least 55%, at least 60%, at least 65%, at least 70%, at least 75%, at least 80%, at least 85%, at least 90%, at least 95%, or at least 99%) of Compound I present in the composition is Compound I mono-citrate Form II.

In one embodiment, provided is a composition comprising 4-(((R)-1-cyanoethyl)amino)-6-(3-cyanopyrrolo[1,2-b]pyridazin-7-yl)-N—((R)-2-fluoro-3-hydroxy-3-methylbutyl)nicotinamide mono-citrate salt Form III (Compound I mono-citrate Form III), or solvate thereof, wherein at least 50% to 99% (e.g., at least 50%, at least 55%, at least 60%, at least 65%, at least 70%, at least 75%, at least 80%, at least 85%, at least 90%, at least 95%, or at least 99%) of Compound I present in the composition is Compound I mono-citrate Form III.

In one embodiment, provided is a composition comprising 4-(((R)-1-cyanoethyl)amino)-6-(3-cyanopyrrolo[1,2-b]pyridazin-7-yl)-N—((R)-2-fluoro-3-hydroxy-3-methylbutyl)nicotinamide mono-citrate salt Form IV (Compound I mono-citrate Form IV), or solvate thereof, wherein at least 50% to 99% (e.g., at least 50%, at least 55%, at least 60%, at least 65%, at least 70%, at least 75%, at least 80%, at least 85%, at least 90%, at least 95%, or at least 99%) of Compound I present in the composition is Compound I mono-citrate Form IV.

In one embodiment, provided is a composition comprising 4-(((R)-1-cyanoethyl)amino)-6-(3-cyanopyrrolo[1,2-b]pyridazin-7-yl)-N—((R)-2-fluoro-3-hydroxy-3-methylbutyl)nicotinamide hemi-citrate salt Form I (Compound I hemi-citrate Form I), or solvate thereof, wherein at least 50% to 99% (e.g., at least 50%, at least 55%, at least 60%, at least 65%, at least 70%, at least 75%, at least 80%, at least 85%, at least 90%, at least 95%, or at least 99%) of Compound I present in the composition is Compound I hemi-citrate Form I.

In one embodiment, provided is a composition comprising 4-(((R)-1-cyanoethyl)amino)-6-(3-cyanopyrrolo[1,2-b]pyridazin-7-yl)-N—((R)-2-fluoro-3-hydroxy-3-methylbutyl)nicotinamide mono-HCl salt Form I (Compound I mono-HCl Form I), or solvate thereof, wherein at least 50% to 99% (e.g., at least 50%, at least 55%, at least 60%, at least 65%, at least 70%, at least 75%, at least 80%, at least 85%, at least 90%, at least 95%, or at least 99%) of Compound I present in the composition is Compound I mono-HCl Form I.

In one embodiment, provided is a composition comprising 4-(((R)-1-cyanoethyl)amino)-6-(3-cyanopyrrolo[1,2-b]pyridazin-7-yl)-N—((R)-2-fluoro-3-hydroxy-3-methylbutyl)nicotinamide mono-maleate salt Form I (Compound I mono-maleate Form I), or solvate thereof, wherein at least 50% to 99% (e.g., at least 50%, at least 55%, at least 60%, at least 65%, at least 70%, at least 75%, at least 80%, at least 85%, at least 90%, at least 95%, or at least 99%) of Compound I present in the composition is Compound I mono-maleate Form I.

In one embodiment, provided is a composition comprising 4-(((R)-1-cyanoethyl)amino)-6-(3-cyanopyrrolo[1,2-b]pyridazin-7-yl)-N—((R)-2-fluoro-3-hydroxy-3-methylbutyl)nicotinamide hemi-fumarate salt Form I (Compound I hemi-fumarate Form I), or solvate thereof, wherein at least 50% to 99% (e.g., at least 50%, at least 55%, at least 60%, at least 65%, at least 70%, at least 75%, at least 80%, at least 85%, at least 90%, at least 95%, or at least 99%) of Compound I present in the composition is Compound I hemi-fumarate Form I.

In one embodiment, provided is a composition comprising 4-(((R)-1-cyanoethyl)amino)-6-(3-cyanopyrrolo[1,2-b]pyridazin-7-yl)-N—((R)-2-fluoro-3-hydroxy-3-methylbutyl)nicotinamide mono-fumarate salt Form I (Compound I mono-fumarate Form I), or solvate thereof, wherein at least 50% to 99% (e.g., at least 50%, at least 55%, at least 60%, at least 65%, at least 70%, at least 75%, at least 80%, at least 85%, at least 90%, at least 95%, or at least 99%) of Compound I present in the composition is Compound I mono-fumarate Form I.

In one embodiment, provided is a composition comprising 4-(((R)-1-cyanoethyl)amino)-6-(3-cyanopyrrolo[1,2-b]pyridazin-7-yl)-N—((R)-2-fluoro-3-hydroxy-3-methylbutyl)nicotinamide hemi-L-tartrate salt Form I (Compound I hemi-L-tartrate Form I), or solvate thereof, wherein at least 50% to 99% (e.g., at least 50%, at least 55%, at least 60%, at least 65%, at least 70%, at least 75%, at least 80%, at least 85%, at least 90%, at least 95%, or at least 99%) of Compound I present in the composition is Compound I hemi-L-tartrate Form I.

In one embodiment, provided is a composition comprising 4-(((R)-1-cyanoethyl)amino)-6-(3-cyanopyrrolo[1,2-b]pyridazin-7-yl)-N—((R)-2-fluoro-3-hydroxy-3-methylbutyl)nicotinamide mono-ESA salt Form I (Compound I mono-ESA Form I), or solvate thereof, wherein at least 50% to 99% (e.g., at least 50%, at least 55%, at least 60%, at least 65%, at least 70%, at least 75%, at least 80%, at least 85%, at least 90%, at least 95%, or at least 99%) of Compound I present in the composition is Compound I mono-ESA Form I.

In one embodiment, provided is a composition comprising 4-(((R)-1-cyanoethyl)amino)-6-(3-cyanopyrrolo[1,2-b]pyridazin-7-yl)-N—((R)-2-fluoro-3-hydroxy-3-methylbutyl)nicotinamide hemi-glycolate salt Form I (Compound I hemi-glycolate Form I), or solvate thereof, wherein at least 50% to 99% (e.g., at least 50%, at least 55%, at least 60%, at least 65%, at least 70%, at least 75%, at least 80%, at least 85%, at least 90%, at least 95%, or at least 99%) of Compound I present in the composition is Compound I hemi-glycolate Form I.

In one embodiment, provided is a composition comprising 4-(((R)-1-cyanoethyl)amino)-6-(3-cyanopyrrolo[1,2-b]pyridazin-7-yl)-N—((R)-2-fluoro-3-hydroxy-3-methylbutyl)nicotinamide sulfate salt Form I (Compound I sulfate Form I), or solvate thereof, wherein at least 50% to 99% (e.g., at least 50%, at least 55%, at least 60%, at least 65%, at least 70%, at least 75%, at least 80%, at least 85%, at least 90%, at least 95%, or at least 99%) of Compound I present in the composition is Compound I sulfate Form I.

In one embodiment, provided is a composition comprising 4-(((R)-1-cyanoethyl)amino)-6-(3-cyanopyrrolo[1,2-b]pyridazin-7-yl)-N—((R)-2-fluoro-3-hydroxy-3-methylbutyl)nicotinamide phosphate salt Form I (Compound I phosphate Form I), or solvate thereof, wherein at least 50% to 99% (e.g., at least 50%, at least 55%, at least 60%, at least 65%, at least 70%, at least 75%, at least 80%, at least 85%, at least 90%, at least 95%, or at least 99%) of Compound I present in the composition is Compound I phosphate Form I.

In one embodiment, provided is a composition comprising 4-(((R)-1-cyanoethyl)amino)-6-(3-cyanopyrrolo[1,2-b]pyridazin-7-yl)-N—((R)-2-fluoro-3-hydroxy-3-methylbutyl)nicotinamide hemi-HCl salt Form I (Compound I hemi-HCl Form I), or solvate thereof, wherein at least 50% to 99% (e.g., at least 50%, at least 55%, at least 60%, at least 65%, at least 70%, at least 75%, at least 80%, at least 85%, at least 90%, at least 95%, or at least 99%) of Compound I present in the composition is Compound I hemi-HCl Form I.

In one embodiment, provided is a composition comprising a crystalline co-crystal of Compound I and 4-(((S)-1-cyanoethyl)amino)-6-(3-cyanopyrrolo[1,2-b]pyridazin-7-yl)-N—((R)-2-fluoro-3-hydroxy-3-methylbutyl)nicotinamide (Compound I co-crystal Form I), or solvate thereof, wherein at least 50% to 99% (e.g., at least 50%, at least 55%, at least 60%, at least 65%, at least 70%, at least 75%, at least 80%, at least 85%, at least 90%, at least 95%, or at least 99%) of Compound I present in the composition is Compound I co-crystal Form I.

In some embodiments, the composition is a pharmaceutical composition which further comprises a pharmaceutically acceptable excipient.

### Pharmaceutical Compositions and Administration

In some embodiments, a chemical entity (e.g., a salt or solid form of Compound I, and salts, co-crystals, solvates, or hydrates thereof, as described herein) that inhibits the kinase IRAK4 is administered as a pharmaceutical composition that includes the chemical entity and one or more pharmaceutically acceptable excipients, and optionally one or more additional therapeutic agents as described herein. Some embodiments provide for a pharmaceutical composition comprising a crystalline form as described herein and a pharmaceutically acceptable carrier.

In some embodiments, the chemical entities can be administered in combination with one or more conventional pharmaceutical excipients. Pharmaceutically acceptable excipients include, but are not limited to, ion exchangers, alumina, aluminum stearate, lecithin, self-emulsifying drug delivery systems (SEDDS) such as d-α-tocopherol polyethylene glycol 1000 succinate, surfactants used in pharmaceutical dosage forms such as Tweens, poloxamers or other similar polymeric delivery matrices, serum proteins, such as human serum albumin, buffer substances such as phosphates, tris, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium-chloride, zinc salts, colloidal silica, magnesium trisilicate, polyvinyl pyrrolidone, cellulose-based substances, polyethylene glycol, sodium carboxymethyl cellulose, polyacrylates, waxes, polyethylene-polyoxypropylene-block polymers, and wool fat. Cyclodextrins such as α-, β, and γ-cyclodextrin, or chemically modified derivatives such as hydroxyalkylcyclodextrins, including 2- and 3-hydroxypropyl-β-cyclodextrins, or other solubilized derivatives can also be used to enhance delivery of chemical entities described herein. Dosage forms or compositions containing a chemical entity as described herein in the range of 0.005% to 100% with the balance made up from non-toxic excipient may be prepared. The contemplated compositions may contain 0.001%-100% of a chemical entity provided herein, in one embodiment 0.1-95%, in another embodiment 75-85%, in a further embodiment 20-80%. Actual methods of preparing such dosage forms are known, or will be apparent, to those skilled in this art; for example, see Remington: The Science and Practice of Pharmacy, 22^{nd} Edition (Pharmaceutical Press, London, U K. 2012).

### Routes of Administration and Composition Components

In some embodiments, the chemical entities described herein or a pharmaceutical composition thereof can be administered to subject in need thereof by any accepted route of administration. Acceptable routes of administration include, but are not limited to, buccal, cutaneous, endocervical, endosinusial, endotracheal, enteral, epidural, interstitial, intra-abdominal, intra-arterial, intrabronchial, intrabursal, intracerebral, intracisternal, intracoronary, intradermal, intraductal, intraduodenal, intradural, intraepidermal, intraesophageal, intragastric, intragingival, intraileal, intralymphatic, intramedullary, intrameningeal, intramuscular, intraovarian, intraperitoneal, intraprostatic, intrapulmonary, intrasinal, intraspinal, intrasynovial, intratesticular, intrathecal, intratubular, intratumoral, intrauterine, intravascular, intravenous, nasal, nasogastric, oral, parenteral, percutaneous, peridural, rectal, respiratory (inhalation), subcutaneous, sublingual, submucosal, topical, transdermal, transmucosal, transtracheal, ureteral, urethral and vaginal.

Compositions can be formulated for parenteral administration, e.g., formulated for injection via the intravenous, intramuscular, sub-cutaneous, or even intraperitoneal routes. Typically, such compositions can be prepared as injectables, either as liquid solutions or suspensions; solid forms suitable for use to prepare solutions or suspensions upon the addition of a liquid prior to injection can also be prepared; and the preparations can also be emulsified. The preparation of such formulations will be known to those of skill in the art in light of the present disclosure.

The pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In general, the form must be sterile and must be fluid to the extent that it may be easily injected. It also should be stable under the conditions of manufacture and storage and must be preserved against the contaminating action of microorganisms, such as bacteria and fungi.

The carrier also can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various antibacterial and antifungal agents, for example, parabens, chlorobutanol, phenol, sorbic acid, thimerosal, and the like. In many cases, isotonic agents, for example, sugars or sodium chloride, may be included. Prolonged absorption of the injectable compositions can be brought about by the use in the compositions of agents delaying absorption, for example, aluminum monostearate and gelatin.

Sterile injectable solutions are prepared by incorporating the active compounds (i.e. the chemical entities described herein) in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, exemplary methods of preparation are vacuum-drying and freeze-drying techniques, which yield a powder of the active ingredient, plus any additional desired ingredient from a previously sterile-filtered solution thereof.

Pharmacologically acceptable excipients usable in the rectal composition as a gel, cream, enema, or rectal suppository, include, without limitation, any one or more of cocoa butter glycerides, synthetic polymers such as polyvinylpyrrolidone, PEG (like PEG ointments), glycerine, glycerinated gelatin, hydrogenated vegetable oils, poloxamers, mixtures of polyethylene glycols of various molecular weights and fatty acid esters of polyethylene glycol Vaseline, anhydrous lanolin, shark liver oil, sodium saccharinate, menthol, sweet almond oil, sorbitol, sodium benzoate, anoxid SBN, vanilla essential oil, aerosol, parabens in phenoxyethanol, sodium methyl p-oxybenzoate, sodium propyl p-oxybenzoate, diethylamine, carbomers, carbopol, methyloxybenzoate, macrogol cetostearyl ether, cocoyl caprylocaprate, isopropyl alcohol, propylene glycol, liquid paraffin, xanthan gum, carboxy-metabisulfite, sodium edetate, sodium benzoate, potassium metabisulfite, grapefruit seed extract, methyl sulfonyl methane (MSM), lactic acid, glycine, vitamins, such as vitamin A and E and potassium acetate.

In certain embodiments, suppositories can be prepared by mixing the chemical entities described herein with suitable non-irritating excipients or carriers such as cocoa butter, polyethylene glycol or a suppository wax which are solid at ambient temperature but liquid at body temperature and therefore melt in the rectum and release the active compound. In other embodiments, compositions for rectal administration are in the form of an enema.

In other embodiments, the chemical entities described herein or a pharmaceutical composition thereof are suitable for local delivery to the digestive or GI tract by way of oral administration (e.g., solid or liquid dosage forms).

Solid dosage forms for oral administration include capsules, tablets, pills, powders, and granules. In such solid dosage forms, the chemical entity is mixed with one or more pharmaceutically acceptable excipients, such as sodium citrate or dicalcium phosphate and/or: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like.

In one embodiment, the compositions will take the form of a unit dosage form such as a pill or tablet and thus the composition may contain, along with a chemical entity provided herein, a diluent such as lactose, sucrose, dicalcium phosphate, or the like; a lubricant such as magnesium stearate or the like; and a binder such as starch, gum acacia, polyvinylpyrrolidine, gelatin, cellulose, cellulose derivatives or the like. In another solid dosage form, a powder, marume, solution or suspension (e.g., in propylene carbonate, vegetable oils, PEG's, poloxamer 124 or triglycerides) is encapsulated in a capsule (gelatin or cellulose base capsule). Unit dosage forms in which one or more chemical entities provided herein or additional active agents are physically separated are also contemplated; e.g., capsules with granules (or tablets in a capsule) of each drug; two-layer tablets; two-compartment gel caps, etc. Enteric coated or delayed release oral dosage forms are also contemplated.

Other physiologically acceptable compounds include wetting agents, emulsifying agents, dispersing agents or preservatives that are particularly useful for preventing the growth or action of microorganisms. Various preservatives are well known and include, for example, phenol and ascorbic acid.

In certain embodiments, the excipients are sterile and generally free of undesirable matter. These compositions can be sterilized by conventional, well-known sterilization techniques. For various oral dosage form excipients such as tablets and capsules, sterility is not required. The USP/NF standard is usually sufficient.

Ocular compositions can include, without limitation, one or more of any of the following: viscogens (e.g., Carboxymethylcellulose, Glycerin, Polyvinylpyrrolidone, Polyethylene glycol); Stabilizers (e.g., Pluronic (triblock copolymers), Cyclodextrins); Preservatives (e.g., Benzalkonium chloride, EDTA, SofZia (boric acid, propylene glycol, sorbitol, and zinc chloride; Alcon Laboratories, Inc.), Purite (stabilized oxychloro complex; Allergan, Inc.)).

Topical compositions can include ointments and creams. Ointments are semisolid preparations that are typically based on petrolatum or other petroleum derivatives. Creams containing the selected active agent are typically viscous liquid or semisolid emulsions, often either oil-in-water or water-in-oil. Cream bases are typically water-washable, and contain an oil phase, an emulsifier and an aqueous phase. The oil phase, also sometimes called the “internal” phase, is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation is generally a nonionic, anionic, cationic or amphoteric surfactant. As with other carriers or vehicles, an ointment base should be inert, stable, nonirritating and non-sensitizing.

In any of the foregoing embodiments, pharmaceutical compositions described herein can include one or more one or more of the following: lipids, interbilayer crosslinked multilamellar vesicles, biodegradeable poly(D,L-lactic-co-glycolic acid) [PLGA]-based or poly anhydride-based nanoparticles or microparticles, and nanoporous particle-supported lipid bilayers.

### Dosages

The dosages may be varied depending on the requirement of the patient, the severity of the condition being treated and the particular compound being employed. Proper dosage for a particular situation can be determined by one skilled in the medical arts. In some cases, the total daily dosage may be divided and administered in portions throughout the day or by means providing continuous delivery.

In some embodiments, the chemical entities described herein are administered at a dosage of from about 0.001 mg/Kg to about 500 mg/Kg (e.g., from about 0.001 mg/Kg to about 200 mg/Kg; from about 0.01 mg/Kg to about 200 mg/Kg; from about 0.01 mg/Kg to about 150 mg/Kg; from about 0.01 mg/Kg to about 100 mg/Kg; from about 0.01 mg/Kg to about 50 mg/Kg; from about 0.01 mg/Kg to about 10 mg/Kg; from about 0.01 mg/Kg to about 5 mg/Kg; from about 0.01 mg/Kg to about 1 mg/Kg; from about 0.01 mg/Kg to about 0.5 mg/Kg; from about 0.01 mg/Kg to about 0.1 mg/Kg; from about 0.1 mg/Kg to about 200 mg/Kg; from about 0.1 mg/Kg to about 150 mg/Kg; from about 0.1 mg/Kg to about 100 mg/Kg; from about 0.1 mg/Kg to about 50 mg/Kg; from about 0.1 mg/Kg to about 10 mg/Kg; from about 0.1 mg/Kg to about 5 mg/Kg; from about 0.1 mg/Kg to about 1 mg/Kg; from about 0.1 mg/Kg to about 0.5 mg/Kg), wherein the Kg refers to the body weight of the patient.

### Regimens

The foregoing dosages can be administered on a daily basis (e.g., as a single dose or as two or more divided doses) or non-daily basis (e.g., every other day, every two days, every three days, once weekly, twice weeks, once every two weeks, once a month).

In some embodiments, the period of administration of a chemical entity described herein is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In an embodiment, a chemical entity described herein is administered to an individual for a period of time followed by a separate period of time. In another embodiment, a chemical entity described herein is administered for a first period and a second period following the first period, with administration stopped during the second period, followed by a third period where administration of the chemical entity described herein is started and then a fourth period following the third period where administration is stopped. In an aspect of this embodiment, the period of administration of a chemical entity described herein followed by a period where administration is stopped is repeated for a determined or undetermined period of time. In a further embodiment, a period of administration is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more.

### Methods of Treatment

This disclosure features methods for treating a subject (e.g., a human) having a disease, disorder, or condition modulated by interleukin-1 receptor-associated kinase-4 (IRAK4).

In some embodiments, the disease, disorder, or condition modulated by IRAK4 is an inflammatory or fibrotic disorder rheumatoid arthritis (RA), inflammatory bowel disease (IBD), gout, Lyme disease, arthritis, psoriasis, pelvic inflammatory disease, systemic lupus erythematosus (SLE), Sjogren's syndrome, inflammation associated with gastrointestinal infections, including C. difficile, viral myocarditis, acute and chronic tissue injury, non-alcoholic steatohepatitis (NASH), alcoholic hepatitis and kidney disease, including chronic kidney disease and diabetic kidney disease.

In some embodiments, the disease, disorder, or condition modulated by IRAK4 is cancer, such as a lymphoma.

In some embodiments, the disease, disorder, or condition modulated by IRAK4 is a metabolic disorder, such as diabetes, including type I and type II diabetes, metabolic syndrome, dyslipidemia, obesity, glucose intolerance, hypertension, elevated serum cholesterol, and elevated triglycerides.

Also provided herein is a method of treating an inflammatory condition, in a patient in need thereof, comprising administering to said patient a salt or solid form of Compound I or a pharmaceutical composition comprising a salt or solid form of Compound I and a pharmaceutically acceptable carrier. Some embodiments provide for a method of treating an inflammatory condition, in a patient in need thereof, comprising administering to said patient a crystalline form as described herein or a pharmaceutical composition as described herein. Some embodiments provide for a method of treating an inflammatory condition, in a patient in need thereof, comprising administering to said patient a therapeutically effective amount of a crystalline form as described herein or a pharmaceutical composition as described herein.

In some embodiments, the inflammatory condition is selected from inflammatory bowel disease (IBD), systemic lupus erythematosus (SLE), psoriasis, and rheumatoid arthritis.

In some embodiments, the inflammatory condition is inflammatory bowel disease (IBD).

In some embodiments, the inflammatory condition is rheumatoid arthritis.

### Combination Therapy

This disclosure contemplates both monotherapy regimens as well as combination therapy regimens.

In some embodiments, the methods described herein can further include administering one or more additional therapies (e.g., one or more additional therapeutic agents and/or one or more therapeutic regimens) in combination with administration of the chemical entities described herein.

In some embodiments, a chemical entity described herein can be administered in combination with one or more of additional therapeutic agents.

Patients being treated by administration of the IRAK4 inhibitors of the disclosure often exhibit diseases or conditions that benefit from treatment with other therapeutic agents. These diseases or conditions can be of an inflammatory nature or can be related to cancer, metabolic disorders, gastrointestinal disorders and the like. Thus, one aspect of the disclosure is a method of treating an inflammation related disease or condition, or a metabolic disorder, gastrointestinal disorder, or cancer and the like comprising administering a chemical entity described herein in combination with one or more compounds useful for the treatment of such diseases to a subject, particularly a human subject, in need thereof.

In some embodiments, a chemical entity described herein is co-formulated with the additional one or more active ingredients. In some embodiments, the other active ingredient is administered at approximately the same time, in a separate dosage form. In some embodiments, the other active ingredient is administered sequentially, and may be administered at different times in relation to a chemical entity described herein.

### Combinations for Inflammatory Diseases and Conditions

For example, a chemical entity of the present disclosure may be combined with one or more 5-Lipoxygenase inhibitors, Acetylcholinesterase inhibitors, Acetyl-CoA carboxylase (ACC) inhibitors, ACTH receptor agonists, Activin receptor antagonists, Acyltransferase inhibitors, Adrenocorticotrophic hormone ligands, AKT1 gene inhibitors, Alkaline phosphatase modulators, Alkaline phosphatase stimulators, Androgen receptor agonists, Apolipoprotein C3 antagonists, ASK1 kinase inhibitors, Bactericidal permeability protein stimulators, Beta adrenoceptor antagonists, Beta-glucuronidase inhibitors, B-lymphocyte antigen CD20 inhibitors, Bradykinin receptor modulators, BTK kinase inhibitors, Calcineurin inhibitors, Calcium channel inhibitors, Cannabinoid CB1 receptor modulators, Cannabinoid CB2 receptor modulators, Cannabinoid receptor antagonists, Cannabinoid receptor modulators, Caspase inhibitors, Cathepsin S inhibitors, CCN protein stimulators, CCR3 chemokine antagonists, CCR5 chemokine antagonists, CCR9 chemokine antagonists, CD3 modulators, CD40 ligand inhibitors, CD40 ligand receptor antagonists, CD49b antagonists, CD49d antagonists, CD89 agonists, Cell adhesion molecule inhibitors, Chemokine CXC ligand inhibitors, CHST15 gene inhibitors, Collagen modulators, CSF-1 agonists, CSF-1 antagonists, CXC10 chemokine ligand inhibitors, CXCR2 chemokine antagonists, Cyclic GMP phosphodiesterase inhibitors, Cyclooxygenase 2 inhibitors, Cyclooxygenase inhibitors, Cyclooxygenase stimulators, Cytochrome P450 3A4 inhibitors, Cytotoxic T-lymphocyte protein-4 stimulators, Dihydroceramide delta 4 desaturase inhibitors, Dihydroorotate dehydrogenase inhibitors, DNA polymerase inhibitors, DPP-4 inhibitors, EGFR family tyrosine kinase receptor modulators, Eosinophil peroxidase inhibitors, Eotaxin ligand inhibitors, EP4 prostanoid receptor agonists, Epidermal growth factor agonists, Epidermal growth factor ligands, Estrogen receptor beta agonists, Factor XIII agonists, FGF-10 ligands, FGF2 receptor agonists, Fractalkine ligand inhibitors, Free fatty acid receptor 2 antagonists, FXR agonists, GATA 3 transcription factor inhibitors, Glucagon-like peptide 1 agonists, Glucagon-like peptide 2 agonists, Glucocorticoid agonists, GM-CSF receptor agonists, G-protein coupled receptor 84 antagonists, Guanylate cyclase receptor agonists, Histamine H2 receptor antagonists, Histone acetyltransferase inhibitors, Histone deacetylase inhibitors, HLA class II antigen modulators, Hydrolase inhibitors, HSD17013 inhibitors, ICAM1 gene inhibitors, ICAM-1 inhibitors, IL1 gene inhibitors, IL-10 agonists, IL10 gene stimulators, IL-11 agonists, IL-12 antagonists, IL12 gene inhibitors, IL-13 antagonists, IL-17 antagonists, IL-2 antagonists, IL-2 receptor alpha subunit inhibitors, IL-21 antagonists, IL-23 antagonists, IL-6 antagonists, IL6 gene inhibitors, IL-6 receptor modulators, IL-7 antagonists, IL-8 antagonists, Immunoglobulin G1 agonists, Immunoglobulin G2 modulators, Inosine monophosphate dehydrogenase inhibitors, Insulin sensitizers, Integrin alpha-4/beta-1 antagonists, Integrin alpha-4/beta-7 antagonists, Integrin alpha-E antagonists, Integrin antagonists, Integrin beta-7 antagonists, Interferon beta ligands, Interleukin 17E ligand inhibitors, Interleukin ligand inhibitors, Interleukin receptor 17A antagonists, Interleukin receptor 17B antagonists, Interleukin-1 beta ligands, Interleukin-1 beta ligand modulators, Interleukin-6 ligand inhibitors, JAK tyrosine kinase inhibitors, Jak1 tyrosine kinase inhibitors, JAK2 gene inhibitors, Jak3 tyrosine kinase inhibitors, Jun N terminal kinase inhibitors, LanC like protein 2 modulators, Leukotriene BLT receptor antagonists, Lipoxygenase modulators, L-Selectin antagonists, MAdCAM inhibitors, Matrix metalloprotease inhibitors, Matrix metalloprotease modulators, Melanocortin agonists, Membrane copper amine oxidase inhibitors, Metalloprotease-2 inhibitors, Metalloprotease-9 inhibitors, MIP 3 alpha ligand inhibitors, Mitochondrial 10 kDa heat shock protein stimulators, Monocyte differentiation antigen CD14 inhibitors, mTOR inhibitors, Mucin stimulators, NAD-dependent deacetylase sirtuin-1 stimulators, Natriuretic peptide receptor C agonists, Neuregulin-4 ligands, Nicotinic acetylcholine receptor agonists, Nicotinic Ach receptor alpha 4 subunit modulators, Nicotinic Ach receptor alpha 7 subunit stimulators, Nicotinic Ach receptor beta 2 subunit modulators, NK1 receptor antagonists, NKG2 D activating NK receptor antagonists, Nuclear factor kappa B inhibitors, Opioid growth factor receptor agonists, Opioid receptor antagonists, Opioid receptor delta antagonists, Oxidoreductase inhibitors, P2X7 purinoceptor agonists, p38 MAP kinase inhibitors, PARP inhibitors, PDE 4 inhibitors, PDGF receptor agonists, Phagocytosis stimulating peptide modulators, Phospho MurNAc pentapeptide transferase inhibitors, Phospholipase A2 inhibitors, Platelet activating factor receptor antagonists, Potassium channel inhibitors, PPAR alpha agonists, PPAR delta agonists, PPAR gamma agonists, Protein CYR61 stimulators, Protein fimH inhibitors, Protein kinase C alpha inhibitors, Protein kinase C beta inhibitors, Protein kinase C delta inhibitors, Protein kinase C epsilon inhibitors, Protein kinase C eta inhibitors, Protein kinase C. theta inhibitors, Protein kinase G inhibitors, Protein kinase inhibitors, P-selectin glycoprotein ligand-1 inhibitors, PurH purine biosynthesis protein inhibitors, Retinoic acid receptor alpha agonists, Retinoic acid receptor beta agonists, Retinoid receptor agonists, RNA polymerase inhibitors, SMAD-7 inhibitors, Sodium channel inhibitors, Somatostatin receptor agonists, Sphingosine 1 phosphate phosphatase 1 stimulators, Sphingosine 1 phosphate phosphatase modulators, Sphingosine kinase 1 inhibitors, Sphingosine kinase 2 inhibitors, Sphingosine-1-phosphate receptor-1 agonists, Sphingosine-1-phosphate receptor-1 antagonists, Sphingosine-1-phosphate receptor-1 modulators, Sphingosine-1-phosphate receptor-5 modulators, STAT3 gene inhibitors, STAT-3 inhibitors, STAT-4 inhibitors, Stem cell antigen-1 inhibitors, Superoxide dismutase modulators, Superoxide dismutase stimulators, SYK kinase inhibitors, T cell surface glycoprotein CD28 inhibitors, TGF beta 1 ligand inhibitors, Thymulin agonists, THR-0 agonists, TLR-2 antagonists, TLR-4 antagonists, TLR-9 agonists, TNF alpha ligand inhibitors, TNF alpha ligand modulators, TNF antagonists, TPL2 kinase inhibitors, Trefoil factor modulators, Tryptase inhibitors, Tryptophan 5-hydroxylase inhibitors, Tumor necrosis factor 14 ligand modulators, TYK2 kinase inhibitors, Type I TNF receptor antagonists, Type II TNF receptor modulators, Unspecified growth factor receptor modulators, Vanilloid VR1 agonists, Vitamin D3 receptor agonists, Zonulin inhibitors, abatacept; acemannan; adalimumab; DCCT-10; apremilast; AST-120; balsalazide; balsalazide sodium; basiliximab; beclomethasone dipropionate; budesonide; D-9421; budesonide MMX; catridecacog; certolizumab pegol; Clostridium butyricum; etanercept; fingolimod; glatiramer acetate; golimumab; infliximab; infliximab biosimilar; infliximab follow-on biologic; interferon beta-la; lenalidomide; mesalazine; GED-0001; AJG-501; metenkefalin acetate with tridecacefamortide acetate, mycophenolate mofetil; naltrexone; natalizumab; nitazoxanide; olsalazine; oprelvekin; propionyl-L-carnitine; recombinant interferon beta-la; remestemcel-L; rifaximin; rituximab; ropivacaine; rosiglitazone; sargramostim; secukinumab; SPD-480; tacrolimus; tamibarotene; teduglutide; thalidomide; tocilizumab; RO-4877533; tofacitinib; CP-690550; Trichuris suis ova; ASP-1002; ustekinumab; valganciclovir; vedolizumab; zileuton; anti-CD3 imaging agent (antibody fragment, cancer/autoimmune disease), ImaginAb; AVX-470; ciclosporin; CXCR1/2 ligands mAb (immunology), Eli Lilly; FFP-102; GSK-3050002; INN-108; IR-777; SGM-1019; peg-ilodecakin; PF-06480605; PF-06651600; SER-287; Syn-1002; Thetanix; tolerogenic dendritic cell therapy TOP-1288; VBY-036; VBY-129; 946414-98-8; BMS-936557; 99mTc-annexin V-128; ABC-294640; abrilumab; Alequel; AMG-139; amiselimod; APD-334; ASP-3291; beclomethasone dipropionate; bertilimumab; ciclosporin; clazakizumab; DLX-105; dolcanatide; E-6011; ETX-201; FFP-104; filgotinib; foralumab; GED-0507-34-Levo; givinostat; GLPG-0974; GLPG-1205; iberogast N (ulcerative colitis), Bayer; BAY98-7410; INV-103; JNJ-40346527; K(D)PT; KAG-308; KHK-4083; KRP-203; larazotide acetate; CB-01-05-MMX; LY-3074828; mesalamine with N-acetylcysteine; midismase; molgramostim follow on biologic with fosfomycin with carbapenem, Reponex; multipotent adult progenitor cell therapy (ischemia/cerebral palsy), Athersys/Healios; NN-8828; olokizumab; OvaSave; P-28-GST; PDA-002; PF-4236921; PF-547659; prednisolone; PUR-0110; QBECO; RBX-2660; repurposed naltrexone; JKB-122; SB-012; sotrastaurin; STNM-01; TAK-114; tetomilast; Debio-0512; TRK-170; TRX-318; vatelizumab; VB-201; ZP-1848; zucapsaicin; ABT-494; alicaforsen; Ampion; BI-655066; briakinumab; cannabidiol; carotegast methyl; cobitolimod; dexamethasone sodium phosphate; elafibranor; etrolizumab; GS-5745; HMPL-004; LP-02; mesalazine; metronidazole mongersen; ocrelizumab; ozanimod; peficitinib; RHB-104; rifaximin; tildrakizumab; tralokinumab; brodalumab; laquinimod; plecanatide; telotristat etiprate; infliximab biosimilar, Samsung Bioepis; AZD-058; and rifabutin with clarithromycin and further with clofazimine.

Also, the following non-exhaustive list of classes of compounds and compounds may be combined with a chemical entity of the present disclosure: 5-Lipoxygenase inhibitors, such as zileuton, etalocibm FPL-64170, E-3040, and BU-4601A; Acetylcholinesterase inhibitors, such as BL-7040; ACTH receptor agonists, such as metenkefalin acetate with tridecactide acetate, and FAR-404; Activin receptor antagonists such as follistatin; Acyltransferase inhibitors such as AZD-0585; Adrenocorticotrophic hormone ligands, such as metenkefalin acetate with tridecactide acetate, and FAR-404; AKT1 gene inhibitors, such as vidofludimus; Alkaline phosphatase modulators such as recombinant human alkaline phosphatase (oral, ulcerative colitis), AM-Pharma; Alkaline phosphatase stimulators such as bovine alkaline phosphatase; Androgen receptor agonists, such as PB-005; Apolipoprotein C3 antagonists, such as AZD-0585; Bactericidal permeability protein stimulators, such as opebacan; Beta adrenoceptor antagonists, such as NM-001; Beta-glucuronidase inhibitors, such as KD-018; B-lymphocyte antigen CD20 inhibitors, such as ocrelizumab, rituximab; Bradykinin receptor modulators, such as givinostat; Calcineurin inhibitors, such as tacrolimus, ciclosporin; Calcium channel inhibitors, such as clotrimazole; Cannabinoid CB1 receptor modulators, such as GWP42003-P, cannabidiol; Cannabinoid CB2 receptor modulators, such as GWP42003-P, cannabidiol; Cannabinoid receptor antagonists, such as fingolimod; Cannabinoid receptor modulators, such as GWP42003-P, cannabidiol; Cathepsin S inhibitors, such as VBY-129, VBY-036; CCN protein stimulators, such as CSA-13; CCR3 chemokine antagonists, such as bertilimumab; CCR5 chemokine antagonists, such as HGS-1025; CCR9 chemokine antagonists, such as MLN-3126, vercirnon, CCX-025; CD3 modulators, such as visilizumab; CD40 ligand inhibitors, such as FFP-104; CD40 ligand receptor antagonists, such as FFP-104, FFP-102, toralizumab; CD49b antagonists, such as vatelizumab; CD49d antagonists, such as ELND-004; CD89 agonists, such as HF-1020; Cell adhesion molecule inhibitors, such as natalizumab, alicaforsen (intravenous), ASP-2002, ISIS-2302; Chemokine CXC ligand inhibitors, such as CXCR1/2 ligands mAb (immunology), Eli Lilly; CHST15 gene inhibitors, such as STNM-01; Collagen modulators, such as adipose-derived stem cell therapy (Celution System), Cytori, DCCT-10; CSF-1 agonists, such as sargramostim, molgramostim follow on biologic with fosfomycin with carbapenem (intraintestinal, Crohn's disease), Reponex; CSF-1 antagonists, such as JNJ-40346527; CXC10 chemokine ligand inhibitors, such as 946414-98-8, BMS-936557; CXCR2 chemokine antagonists, such as elubrixin; Cyclic GMP phosphodiesterase inhibitors, such as CEL-031; Cyclooxygenase 2 inhibitors, such as P-54; Cyclooxygenase inhibitors, such as mesalazine, 4-aminosalicylate sodium, AJG-501, AGI-022; Cyclooxygenase stimulators, such as nicotine polacrilex; Cytochrome P450 3A4 inhibitors, such as KD-018; Cytotoxic T-lymphocyte protein-4 stimulators, such as abatacept; Dihydroceramide delta 4 desaturase inhibitors, such as ABC-294640; Dihydroorotate dehydrogenase inhibitors, such as vidofludimus; DNA polymerase inhibitors, such as valganciclovir; EGFR family tyrosine kinase receptor modulators, such as neuregulin 4 (Crohn's disease/ulcerative colitis/necrotizing enterocolitis), Avexegen Therapeutics/Children's Hospital of Los Angeles; Eosinophil peroxidase inhibitors, such as AWEPOPD-01, AWEPO-003; Eotaxin ligand inhibitors, such as bertilimumab; EP4 prostanoid receptor agonists, such as KAG-308; Epidermal growth factor agonists, such as heparin-EGF-like factor, Scios Nova; Epidermal growth factor ligands, such as Hebervis; Estrogen receptor beta agonists, such as prinaberel; Factor XIII agonists, such as catridecacog; FGF-10 ligands, such as repifermin; FGF2 receptor agonists, such as F2A; Fractalkine ligand inhibitors, such as E-6011; Free fatty acid receptor 2 antagonists, such as GLPG-0974; GATA 3 transcription factor inhibitors, such as SB-012; Glucagon-like peptide 2 agonists, such as teduglutide, ZP-1848, NB-1002; Glucocorticoid agonists, such as budesonide, beclomethasone dipropionate, dexamethasone sodium phosphate, AJG-511, DOR-201, D-9421-C; GM-CSF receptor agonists, such as sargramostim, molgramostim follow on biologic with fosfomycin with carbapenem (intraintestinal, Crohn's disease), Reponex; G-protein coupled receptor 84 antagonists, such as GLPG-1205; Guanylate cyclase receptor agonists, such as dolcanatide, SP-333; Histamine H2 receptor antagonists, such as bismuth, Medeva; Histone acetyltransferase inhibitors, such as TIP60 inhibitors (ulcerative colitis/inflammatory bowel disease/autoimmune diseases), University of Pennsylvania; Histone deacetylase inhibitors, such as givinostat; HLA class II antigen modulators, such as HLA class II protein modulators (Crohns disease), NexteraAS; Hydrolase inhibitors, such as SC-56938; ICAM1 gene inhibitors, such as alicaforsen; ICAM-1 inhibitors, such as alicaforsen (intravenous), ISIS-2302; IL1 gene inhibitors, such as PLR-14; IL-10 agonists, such as peg-ilodecakin, AM-0010; IL10 gene stimulators, such as gene therapy (IL-10), Imperial College; IL-11 agonists, such as oprelvekin, YM-294; IL-12 antagonists, such as ustekinumab, briakinumab, apilimod; IL12 gene inhibitors, such as RDP-58; IL-13 antagonists, such as tralokinumab, anrukinzumab; IL-17 antagonists, such as secukinumab, vidofludimus; IL-2 antagonists, such as daclizumab; IL-2 receptor alpha subunit inhibitors, such as basiliximab, daclizumab, BSX-003, Ro-34-7375; IL-21 antagonists, such as NN-8828, ATR-107; IL-23 antagonists, such as tildrakizumab, ustekinumab, BI-655066, AMG-139, briakinumab, LY-3074828, apilimod; IL-6 antagonists, such as tocilizumab, clazakizumab, olokizumab, HMPL-004, AMG-220, FM-101; IL6 gene inhibitors, such as YSIL6-T-PS; IL-6 receptor modulators, such as tocilizumab; IL-7 antagonists, such as interleukin-7 receptor modulators (ulcerative colitis/T-cell acute lymphoblastic leukaemia), Effimune; IL-8 antagonists, such as elubrixin, clotrimazole; Immunoglobulin G1 agonists, such as HF-1020; Immunoglobulin G2 modulators, such as PF-547659; Inosine monophosphate dehydrogenase inhibitors, such as mycophenolate mofetil; Insulin sensitizers, such as elafibranor, rosiglitazone, HE-3286, EGS-21; Integrin alpha-4/beta-1 antagonists, such as natalizumab, TRK-170, firategrast; Integrin alpha-4/beta-7 antagonists, such as etrolizumab, vedolizumab, abrilumab, carotegast methyl, TRK-170, firategrast; Integrin alpha-E antagonists, such as etrolizumab; Integrin antagonists, such as vatelizumab, ASP-2002; Integrin beta-7 antagonists, such as etrolizumab; Interferon beta ligands, such as interferon beta-la, recombinant interferon beta-la, Serono; Interleukin 17E ligand inhibitors, such as anti-IL-17BR humanized antibody (lung fibrosis/asthma/ulcerative colitis), Medical Research Council Technology; Interleukin ligand inhibitors, such as HE-3286; Interleukin receptor 17A antagonists, such as brodalumab; Interleukin receptor 17B antagonists, such as anti-IL-17BR humanized antibody (lung fibrosis/asthma/ulcerative colitis), Medical Research Council Technology; Interleukin-1 beta ligands, such as K(D)PT, PUR-0110, HMPL-004; Interleukin-1 beta ligand modulators, such as PUR-0110, HMPL-004; Interleukin-6 ligand inhibitors, such as PF-4236921; JAK tyrosine kinase inhibitors, such as tofacitinib, peficitinib; Jak1 tyrosine kinase inhibitors, such as ABT-494, tofacitinib, filgotinib, peficitinib, GLPG-0555, solcitinib; JAK2 gene inhibitors, such as vidofludimus; Jak3 tyrosine kinase inhibitors, such as tofacitinib, peficitinib; Jun N terminal kinase inhibitors, such as semapimod; LanC like protein 2 modulators, such as BT-11; Leukotriene BLT receptor antagonists, such as ONO-4057, etalocib, SC-53228, SC-52798; Lipoxygenase modulators, such as mesalazine; L-Selectin antagonists, such as BNP-001; MAdCAM inhibitors, such as vedolizumab, PF-547659; Matrix metalloprotease inhibitors, such as D-5410; Matrix metalloprotease modulators, such as D-5410; Melanocortin agonists, such as ASP-3291; Membrane copper amine oxidase inhibitors, such as vepalimomab; Metalloprotease-2 inhibitors, such as KD-018, RWJ-68354; Metalloprotease-9 inhibitors, such as GS-5745; MIP 3 alpha ligand inhibitors, such as GSK-3050002; Mitochondrial 10 kDa heat shock protein stimulators, such as INV-103; Monocyte differentiation antigen CD14 inhibitors, such as CD14 anti-inflammatory, Cornell; mTOR inhibitors, such as P-2281; Mucin stimulators, such as rebamipide; NAD-dependent deacetylase sirtuin-1 stimulators, such as SRT-2104; Natriuretic peptide receptor C agonists, such as plecanatide; Neuregulin-4 ligands, such as neuregulin 4 (Crohn's disease/ulcerative colitis/necrotizing enterocolitis), Avexegen Therapeutics/Children's Hospital of Los Angeles; Nicotinic acetylcholine receptor agonists, such as TC-2403, nicotine polacrilex, nicotine; Nicotinic Ach receptor alpha 4 subunit modulators, such as TC-2403; Nicotinic Ach receptor alpha 7 subunit stimulators, such as GTS-21; Nicotinic Ach receptor beta 2 subunit modulators, such as TC-2403; NK1 receptor antagonists, such as KD-018, nolpitantium besilate; NKG2 D activating NK receptor antagonists, such as NNC-0142-002; Nuclear factor kappa B inhibitors, such as KD-018, cobitolimod, CSA-13, HE-3286, HMPL-004, Avrina, mesalamine with N-acetylcysteine, P-54; Opioid growth factor receptor agonists, such as metenkefalin acetate with tridecactide acetate, FAR-404; Opioid receptor antagonists, such as naltrexone, IRT-103; Opioid receptor delta antagonists, such as KD-018; Oxidoreductase inhibitors, such as olsalazine; P2X7 purinoceptor agonists, such as givinostat; p38 MAP kinase inhibitors, such as RDP-58, doramapimod, semapimod, RWJ-68354; PARP inhibitors, such as EB-47, INO-1003; PDE 4 inhibitors, such as apremilast, tetomilast, CC-1088; PDGF receptor agonists, such as oprelvekin, YM-294; Phagocytosis stimulating peptide modulators, such as 99mTc-RP-128; Phospho MurNAc pentapeptide transferase inhibitors, such as SQ-641; Phospholipase A2 inhibitors, such as varespladib methyl; Platelet activating factor receptor antagonists, such as dersalazine sodium; Potassium channel inhibitors, such as clotrimazole; PPAR alpha agonists, such as elafibranor (GFT-1007); PPAR delta agonists, such as elafibranor (GFT-1007); PPAR gamma agonists, such as rosiglitazone, GED-0507-34-Levo, etalocib; Protein CYR61 stimulators, such as CSA-13; Protein fimH inhibitors, such as EB-8018; Protein kinase C alpha inhibitors, such as sotrastaurin (AEB-071); Protein kinase C beta inhibitors, such as sotrastaurin (AEB-071); Protein kinase C delta inhibitors, such as sotrastaurin (AEB-071); Protein kinase C epsilon inhibitors, such as sotrastaurin (AEB-071); Protein kinase C eta inhibitors, such as sotrastaurin (AEB-071); Protein kinase C. theta inhibitors, such as sotrastaurin (AEB-071); Protein kinase G inhibitors, such as CEL-031; Protein kinase inhibitors, such as TOP-1288; P-selectin glycoprotein ligand-1 inhibitors, such as SEL-K2; PurH purine biosynthesis protein inhibitors, such as mycophenolate mofetil; Retinoic acid receptor alpha agonists, such as tamibarotene; Retinoic acid receptor beta agonists, such as tamibarotene; Retinoid receptor agonists, such as tamibarotene; RNA polymerase inhibitors, such as rifaximin; SMAD-7 inhibitors, such as mongersen (GED-0301); Sodium channel inhibitors, such as ropivacaine; Somatostatin receptor agonists, such as vapreotide; Sphingosine 1 phosphate phosphatase 1 stimulators, such as APD-334; Sphingosine 1 phosphate phosphatase modulators, such as SIP modulators (oral, multiple sclerosis/ulcerative colitis/rheumatoid arthritis), Akaal Pharma; Sphingosine kinase 1 inhibitors, such as ABC-294640; Sphingosine kinase 2 inhibitors, such as ABC-294640; Sphingosine-1-phosphate receptor-1 agonists, such as ozanimod (RPC-1063), KRP-203; Sphingosine-1-phosphate receptor-1 antagonists, such as amiselimod (MT-1303); Sphingosine-1-phosphate receptor-1 modulators, such as fingolimod (FTY-720), ozanimod (RPC-1063), amiselimod (MT-1303); Sphingosine-1-phosphate receptor-5 modulators, such as ozanimod; STAT3 gene inhibitors, such as vidofludimus; STAT-3 inhibitors, such as TAK-114; STAT-4 inhibitors, such as STAT-4 antisense oligonucleotide (Crohns disease/colitis), NIAID; Stem cell antigen-1 inhibitors, such as Ampion, DMI-9523; Superoxide dismutase modulators, such as midismase, LT-0011; Superoxide dismutase stimulators, such as superoxide dismutase; T cell surface glycoprotein CD28 inhibitors, such as abatacept; TGF beta 1 ligand inhibitors, such as mongersen, GED-0301; Thymulin agonists, such as Syn-1002; TLR-2 antagonists, such as VB-201; TLR-4 antagonists, such as JKB-122, VB-201; TLR-9 agonists, such as BL-7040, cobitolimod; TNF alpha ligand inhibitors, such as adalimumab, certolizumab pegol, infliximab biosimilar, infliximab, golimumab, ISIS-104838, CSA-13, DLX-105, adalimumab biosimilar, dersalazine sodium, Debio-0512, HMPL-004, DLX-105, infliximab follow-on biologic, AZD-9773, CYT-020-TNFQb, DOM-0200; TNF alpha ligand modulators, such as PUR-0110, CDP-571; TNF antagonists, such as etanercept, certolizumab pegol, AVX-470, onercept; Trefoil factor modulators, such as AG-012; Tryptase inhibitors, such as APC-2059; Tryptophan 5-hydroxylase inhibitors, such as telotristat etiprate; Tumor necrosis factor 14 ligand modulators, such as SAR-252067; Type I TNF receptor antagonists, such as DOM-0100; Type II TNF receptor modulators, such as etanercept; Unspecified growth factor receptor modulators, such as AP-005; Vanilloid VR1 agonists, such as zucapsaicin; Vitamin D3 receptor agonists, such as calcitriol; and Zonulin inhibitors, such as larazotide acetate, AT-1001.

Also, the following non-exhaustive list of classes of compounds and compounds may be combined with a chemical entity of the present disclosure: 14-3-3 protein eta inhibitors, 5-Lipoxygenase inhibitors, Abl tyrosine kinase inhibitors, ACTH receptor agonists, Adenosine A3 receptor agonists, Adenosine deaminase inhibitors, ADP ribosyl cyclase-1 modulators, ADP ribosylation factor 6 inhibitors, Adrenocorticotrophic hormone ligands, Aggrecanase-2 inhibitors, Albumin modulators, AP1 transcription factor inhibitors, Basigin inhibitors, Bcr protein inhibitors, B-lymphocyte antigen CD19 inhibitors, B-lymphocyte antigen CD20 inhibitors, B-lymphocyte antigen CD20 modulators, B-lymphocyte stimulator ligand inhibitors, Bradykinin receptor modulators, BRAF gene inhibitors, Branched amino acid aminotransferase 1 inhibitors, Bromodomain containing protein inhibitors, Btk tyrosine kinase inhibitors, Cadherin-11 antagonists, Calcineurin inhibitors, Calcium channel inhibitors, Carbonic anhydrase inhibitors, Cathepsin K inhibitors, Cathepsin S inhibitors, CCR1 chemokine antagonists, CCR2 chemokine antagonists, CCR3 gene modulators, CCR5 chemokine antagonists, CD126 antagonists, CD29 modulators, CD3 modulators, CD39 agonists, CD4 agonists, CD4 antagonists, CD40 ligand inhibitors, CD40 ligand receptor antagonists, CD40 ligand receptor modulators, CD52 antagonists, CD73 agonists, CD79b modulators, CD80 antagonists, CD86 antagonists, CD95 antagonists, Cell adhesion molecule inhibitors, Choline kinase inhibitors, Clusterin stimulators, Complement C5 factor inhibitors, Complement Factor stimulators, C-reactive protein inhibitors, CSF-1 antagonists, CXC10 chemokine ligand inhibitors, CXCR4 chemokine antagonists, Cyclin-dependent kinase inhibitor 1 inhibitors, Cyclin-dependent kinase-2 inhibitors, Cyclin-dependent kinase-4 inhibitors, Cyclin-dependent kinase-5 inhibitors, Cyclin-dependent kinase-6 inhibitors, Cyclin-dependent kinase-7 inhibitors, Cyclin-dependent kinase-9 inhibitors, Cyclooxygenase 2 inhibitors, Cyclooxygenase 2 modulators, Cyclooxygenase inhibitors, Cytosolic phospholipase A2 inhibitors, Cytotoxic T-lymphocyte protein-4 modulators, Cytotoxic T-lymphocyte protein-4 stimulators, DHFR inhibitors, Diamine acetyltransferase inhibitors, Dihydroorotate dehydrogenase inhibitors, Elongation factor 2 inhibitors, Eotaxin 2 ligand inhibitors, EP4 prostanoid receptor antagonists, Erythropoietin receptor agonists, Fas ligands, FGF-2 ligand inhibitors, FK506 binding protein-12 modulators, Folate antagonists, Folate receptor agonists, Folate receptor beta antagonists, Folate receptor modulators, Fractalkine ligand inhibitors, Fyn tyrosine kinase inhibitors, G protein coupled receptor 15 antagonists, GABAA receptor modulators, Glucocorticoid agonists, Glucocorticoid antagonists, Glucocorticoid induced leucine zipper stimulators, GM-CSF ligand inhibitors, GM-CSF receptor antagonists, GM-CSF receptor modulators, Growth regulated protein alpha ligand inhibitors, Hwith Kwith ATPase inhibitors, Histamine H4 receptor antagonists, Histone deacetylase inhibitors, Histone deacetylase-6 inhibitors, HIV-1 gp120 protein inhibitors, HLA class II antigen DQ-2 alpha modulators, HLA class II antigen inhibitors, HLA class II antigen modulators, Hsp 70 family inhibitors, Hypoxia inducible factor-1 inhibitors, IFNB gene stimulators, I-kappa B kinase beta inhibitors, I-kappa B kinase inhibitors, IL-1 antagonists, IL-10 agonists, IL-11 agonists, IL-12 antagonists, IL-15 antagonists, IL-17 antagonists, IL-17 receptor modulators, IL-2 agonists, IL-2 antagonists, IL-21 antagonists, IL-23 antagonists, IL-3 antagonists, IL-4 agonists, IL-6 antagonists, IL-6 receptor modulators, Immunoglobulin antagonists, Immunoglobulin G1 agonists, Immunoglobulin G1 antagonists, Immunoglobulin G1 modulators, Immunoglobulin G2 antagonists, Immunoglobulin G2 modulators, Immunoglobulin gamma Fc receptor II modulators, Immunoglobulin gamma Fc receptor IIB antagonists, Immunoglobulin kappa modulators, Immunoglobulin M antagonists, Inducible nitric oxide synthase inhibitors, Inosine monophosphate dehydrogenase inhibitors, Insulin sensitizers, Integrin alpha-1/beta-1 antagonists, Integrin alpha-4/beta-1 antagonists, Integrin antagonists, Interferon beta ligands, Interferon gamma ligands, Interleukin 17A ligand inhibitors, Interleukin 17F ligand inhibitors, Interleukin 23A inhibitors, Interleukin ligands, Interleukin receptor 17A antagonists, Interleukin-1 beta ligand inhibitors, Interleukin-10 ligands, Interleukin-2 ligands, Interleukin-4 ligands, Interleukin-6 ligand inhibitors, Itk tyrosine kinase inhibitors, JAK tyrosine kinase inhibitors, Jak1 tyrosine kinase inhibitors, Jak2 tyrosine kinase inhibitors, JAK3 gene inhibitors, Jak3 tyrosine kinase inhibitors, Jun N terminal kinase inhibitors, KCNA voltage-gated potassium channel-3 modulators, Kelch like ECH associated protein 1 modulators, Kit tyrosine kinase inhibitors, LanC like protein 2 modulators, LITAF gene inhibitors, Lymphocyte function antigen-3 receptor antagonists, Lyn tyrosine kinase inhibitors, Macrophage mannose receptor 1 modulators, MAdCAM inhibitors, MAP kinase modulators, MAP3K2 gene inhibitors, MAPKAPK5 inhibitors, Matrix metalloprotease inhibitors, MCL1 gene inhibitors, MEK protein kinase inhibitors, MEK-1 protein kinase inhibitors, MEK-2 protein kinase inhibitors, Membrane copper amine oxidase inhibitors, Metalloprotease-2 inhibitors, Metalloprotease-9 inhibitors, Midkine ligand inhibitors, Mitochondrial 10 kDa heat shock protein stimulators, mTOR complex 1 inhibitors, mTOR inhibitors, NAD ADP ribosyltransferase stimulators, NAMPT gene inhibitors, NF kappa B inhibitor stimulators, NFAT gene inhibitors, NFE2L2 gene stimulators, Nicotinic acetylcholine receptor antagonists, NK cell receptor modulators, NKG2 A B activating NK receptor antagonists, NKG2 D activating NK receptor antagonists, Nuclear erythroid 2-related factor 2 stimulators, Nuclear factor kappa B inhibitors, Nuclear factor kappa B modulators, Nuclear factor kappa B p105 inhibitors, Opioid growth factor receptor agonists, Opioid receptor delta antagonists, Osteoclast differentiation factor antagonists, Osteoclast differentiation factor ligand inhibitors, Oxidoreductase inhibitors, P2X7 purinoceptor agonists, p38 MAP kinase alpha inhibitors, p38 MAP kinase inhibitors, PDE 4 inhibitors, PDE 5 inhibitors, PDGF receptor agonists, PDGF receptor antagonists, PDGF-B ligand inhibitors, PERK gene inhibitors, Phosphoinositide-3 kinase delta inhibitors, Phosphoinositide-3 kinase gamma inhibitors, Phospholipase A2 inhibitors, Platelet activating factor receptor antagonists, PPAR gamma agonists, Programmed cell death protein 1 modulators, Prostaglandin D synthase stimulators, Protein arginine deiminase inhibitors, Protein tyrosine kinase inhibitors, PurH purine biosynthesis protein inhibitors, Rho associated protein kinase 2 inhibitors, Seprase inhibitors, Signal transducer CD24 modulators, Signal transduction inhibitors, Sodium glucose transporter-2 inhibitors, Sphingosine 1 phosphate phosphatase modulators, STAT3 gene inhibitors, Superoxide dismutase stimulators, SYK family tyrosine kinase inhibitors, Syk tyrosine kinase inhibitors, Syndecan-1 inhibitors, T cell receptor antagonists, T cell receptor modulators, T cell surface glycoprotein CD28 inhibitors, T cell surface glycoprotein CD28 stimulators, TAK1 binding protein modulators, Talin modulators, T-cell differentiation antigen CD6 inhibitors, T-cell surface glycoprotein CD8 inhibitors, Tenascin modulators, TGF beta agonists, Thymulin agonists, TLR-2 antagonists, TLR-4 antagonists, TLR-9 antagonists, TNF alpha ligand inhibitors, TNF alpha ligand modulators, TNF antagonists, TNF gene inhibitors, TNF receptor modulators, TNFSF11 gene inhibitors, Transcription factor p65 inhibitors, Transcription factor RelB inhibitors, Transferrin modulators, Tumor necrosis factor 13C receptor antagonists, Tumor necrosis factor 15 ligand inhibitors, Tumor necrosis factor ligand 13 inhibitors, Tumor necrosis factor ligand inhibitors, Type I IL-1 receptor antagonists, Type I TNF receptor antagonists, Type II TNF receptor modulators, Unspecified GPCR agonists, VEGF receptor antagonists, VEGF-2 receptor antagonists, VEGF-2 receptor modulators, VEGF-B ligand inhibitors, X-linked inhibitor of apoptosis protein inhibitors, Zap70 tyrosine kinase inhibitors, 99mTc labelled annexin V-128, abatacept, abatacept biosimilar, ABBV-257, ABT-122, ABT-494, acalabrutinib, aceclofenac, actarit, MS-392, adalimumab, adalimumab biosimilar, adalimumab follow-on biologic, AK-106, ALX-0061, aminopterin, anakinra, anakinra biosimilar, anakinra follow-on biologic, ARG-301, ASLAN-003, ASP-5094, AT-132, AZD-9567, baricitinib, BI-655064, bimekizumab, BiP (rheumatoid arthritis), Kings College London, BLHP-006, blisibimod, BMS-986104, BMS-986142, ABBV-105, BTT-1023, canakinumab, Cartistem, CCX-354, CD24-IgFc, celecoxib, cerdulatinib, certolizumab pegol, CF-101, CFZ-533, CHR-5154, cibinetide, ciclosporin, clazakizumab, CNTO-6785, corticotropin, Mallinckrodt, CR-6086, CreaVax-RA, CWG-92, CWG-940, Cx-611, DE-098, deflazacort, Rheumavax, denosumab, diacerein, diclofenac, E-6011, eicosapentaenoic acid monoglycerides, etanercept, etanercept biosimilar, etanercept follow-on biologic, etodolac, etoricoxib, filgotinib, fosdagrocorat, gerilimzumab, ginsenoside C—K, givinostat, goat polyclonal antibodies, golimumab, GS-5745, GS-9876, GSK-3196165, HM-71224, HMPL-523, hyaluronate sodium, IB-RA (injectable, rheumatoid arthritis), Innobioscience, IB-RA (oral, rheumatoid arthritis), Innobioscience, iguratimod, IMD-2560, imidazole salicylate, infliximab, infliximab biobetter, infliximab biosimilar, INSIX RA, interferon gamma follow-on biologic, interleukin-2 (injectable), interleukin-2 follow-on biologic, INV-103, IR-501, itolizumab, JNJ-40346527, Ka Shu Ning, KD-025, ketoprofen with omeprazole, leflunomide, lenzilumab, LLDT-8, lumiracoxib, LY-3090106, masitinib, mavrilimumab, MBS-2320, MEDI-5117, meloxicam, methotrexate, MGD-010, misoprostol with diclofenac, MM-A01-01, monalizumab, MORAb-022, MPC-300-IV, MRC-375, nabumetone, namilumab, naproxen with esomeprazole, naproxen with esomeprazole strontium, ocaratuzumab, ofatumumab, OHR-118, olokizumab, OM-89, once-daily naproxen (oral controlled release, pain), Alvogen, ONO-4059, Oralgam, ozoralizumab, peficitinib, pelubiprofen, PF-06687234, piperidone hydrochloridum, piroxicam, prednisolone, prednisone, Prosorba, PRT-2607, PRTX-100, PRX-167700, QBSAU, rabeximod, RCT-18, recombinant human CD22 monoclonal antibody (iv infusion), Lonn Ryonn Pharma/SinoMab Bioscience (Shenzhen), recombinant human interleukin-1 receptor antagonist (rheumatoid arthritis), Shanghai Fudan-Zhangjiang Bio-Pharmaceutical, recombinant human interleukin-2 recombinant TNF receptor 2-Fc fusion protein mutant, RG-6125, RhuDex, rifabutin with clarithromycin with clofazimine, rituximab, rituximab biosimilar, rituximab follow-on biologic, RPI-78, SAN-300, sarilumab, SBI-087, seliciclib, SHR-0302, sirukumab, spebrutinib, SSS-07, KDDF-201110-06, Syn-1002, T-5224, TAB-08, tacrolimus, TAK-020, TAK-079, tarenflurbil (transdermal spraygel, skin disease/rheumatoid arthritis), MIKA Pharma/GALENpharma, technetium Tc 99m tilmanocept, technetium[99Tc] methylenediphosphonate, tenoxicam, Debio-0512, tocilizumab, tofacitinib, Trichuris suis ova, umbilical cord-derived mesenchymal stem cells (iv, RA/liver disease), Alliancells/Zhongyuan Union, ustekinumab, VAY-736, VB-201, WF-10, XmAb-5871, YHB-1411-2; 14-3-3 protein eta inhibitors, such as anti-AGX-020 mAbs (rheumatoid arthritis), Augurex; 5-Lipoxygenase inhibitors, such as tenoxicam, darbufelone, tebufelone, licofelone, ZD-2138, etalocib, tenidap, tepoxalin, flobufen, SKF-86002, PGV-20229, L-708780, WY-28342, T-0757, T-0799, ZM-216800, L-699333, BU-4601A, SKF-104351, CI-986; Abl tyrosine kinase inhibitors, such as imatinib; ACTH receptor agonists, such as FAR-404, metenkefalin acetate with tridecactide acetate; Adenosine A3 receptor agonists, such as CF-101; Adenosine deaminase inhibitors, such as cladribine, pentostatin, FR-221647; ADP ribosyl cyclase-1 modulators, such as indatuximab ravtansine; ADP ribosylation factor 6 inhibitors, such as NAV-2729; Adrenocorticotrophic hormone ligands, such as corticotropin, Mallinckrodt, FAR-404, metenkefalin acetate with tridecactide acetate; Aggrecanase-2 inhibitors, such as GIBH-R-001-2; Albumin modulators, such as ALX-0061, ONS-1210; API transcription factor inhibitors, such as T-5224, tarenflurbil, SP-10030; Basigin inhibitors, such as ERG-240; Bcr protein inhibitors, such as imatinib; B-lymphocyte antigen CD19 inhibitors, such as XmAb-5871, MDX-1342; B-lymphocyte antigen CD20 inhibitors, such as ocrelizumab, ofatumumab, rituximab, rituximab biosimilar, veltuzumab, rituximab follow-on biologic, ocaratuzumab, BLX-301, IDEC-102, ABP-798, GP-2013, MK-8808, HLX-01, CT-P10, TL-011, PF-05280586, IBPM-001RX, IBI-301, AME-133v, BCD-020, BT-D004, SAIT-101; B-lymphocyte antigen CD20 modulators, such as rituximab biosimilar, SBI-087, TRU-015, DXL-625; B-lymphocyte stimulator ligand inhibitors, such as belimumab, RCT-18, blisibimod, tabalumab, atacicept, briobacept; Bradykinin receptor modulators, such as givinostat; BRAF gene inhibitors, such as binimetinib; Branched amino acid aminotransferase 1 inhibitors, such as ERG-240; Bromodomain containing protein inhibitors, such as RVX-297, ZEN-003694; Btk tyrosine kinase inhibitors, such as acalabrutinib, HM-71224, spebrutinib, BTK inhibitor (rheumatoid arthritis), Humanwell Healthcare/Wuxi AppTech, BMS-986142, TAK-020, ONO-4059, TAS-5315, ABBV-105, AC-0025, RN-486, CG-026806, GDC-0834; Cadherin-11 antagonists, such as RG-6125; Calcineurin inhibitors, such as HS-378, ciclosporin; Calcium channel inhibitors, such as RP-3128; Carbonic anhydrase inhibitors, such as polmacoxib; Cathepsin K inhibitors, such as CRA-013783, T-5224, AM-3876, VEL-0230, NPI-2019; Cathepsin S inhibitors, such as MIV-247, AM-3876, RWJ-445380, NPI-2019; CCR1 chemokine antagonists, such as BX-471, BMS-817399, BI-638683, CCX-354, MLN-3701, MLN-3897, CP-481715, PS-375179; CCR2 chemokine antagonists, such as MK-0812, AZD-6942; CCR3 gene modulators, such as CM-102; CCR5 chemokine antagonists, such as maraviroc, OHR-118, NIBR-6465, AZD-5672, AZD-8566; CD126 antagonists, such as sarilumab; CD29 modulators, such as PF-06687234; CD3 modulators, such as otelixizumab; CD39 agonists, such as AAVS-CD39/CD73 (rheumatoid arthritis), Arthrogen; CD4 agonists, such as maraviroc; CD4 antagonists, such as tregalizumab, zanolimumab, MTRX-1011A, BW-4162W94, EP-1645, clenoliximab; CD40 ligand inhibitors, such as dapirolizumab pegol; CD40 ligand receptor antagonists, such as BI-655064, anti-CD40-XTEN, teneliximab; CD40 ligand receptor modulators, such as CFZ-533; CD52 antagonists, such as alemtuzumab; CD73 agonists, such as AAVS-CD39/CD73 (rheumatoid arthritis), Arthrogen; CD79b modulators, such as MGD-010; CD80 antagonists, such as RhuDex, XENP-9523, ASP-2408, abatacept biobetter; CD86 antagonists, such as ES-210, abatacept biosuperior, ASP-2408, XENP-9523; CD95 antagonists, such as DE-098, CS-9507; Cell adhesion molecule inhibitors, such as natalizumab, alicaforsen, NPC-17923, TK-280, PD-144795; Choline kinase inhibitors, such as choline kinase inhibitors (rheumatoid arthritis), UC San Diego; Clusterin stimulators, such as alemtuzumab; Complement C5 factor inhibitors, such as eculizumab, antisense oligonucleotides (rheumatoid arthritis), Leiden University Medical Center; Complement Factor stimulators, such as CM-101; C-reactive protein inhibitors, such as IB-RA (oral, rheumatoid arthritis), Innobioscience, ISIS-353512; CSF-1 antagonists, such as masitinib, FPA-008, JNJ-27301937, JNJ-40346527, PLX-5622, CT-1578, PD-360324, JNJ-28312141; CXC10 chemokine ligand inhibitors, such as 946414-98-8, BMS-936557; CXCR4 chemokine antagonists, such as plerixafor; Cyclin-dependent kinase inhibitor 1 inhibitors, such as CDK-1/2/5/7/9 inhibitors (cancer/tumorogenesis/rheumatoid arthritis), BioPatterns; Cyclin-dependent kinase-2 inhibitors, such as seliciclib, BP-14; Cyclin-dependent kinase-4 inhibitors, such as CDK-4/6 inhibitor (rheumatoid arthritis), Teijin; Cyclin-dependent kinase-5 inhibitors, such as BP-14; Cyclin-dependent kinase-6 inhibitors, such as CDK-4/6 inhibitor (rheumatoid arthritis), Teijin; Cyclin-dependent kinase-7 inhibitors, such as BP-14, seliciclib; Cyclin-dependent kinase-9 inhibitors, such as BP-14, seliciclib; Cyclooxygenase 2 inhibitors, such as celecoxib, etoricoxib, polmacoxib, laflunimus, etodolac, meloxicam, IB-RA (injectable, rheumatoid arthritis), Innobioscience, IB-RA (oral, rheumatoid arthritis), Innobioscience, SKLB-023, meloxicam, lumiracoxib; Cyclooxygenase 2 modulators, such as DRGT-46; Cyclooxygenase inhibitors, such as aceclofenac, diclofenac, imidazole salicylate, naproxcinod, naproxen etemesil, misoprostol with diclofenac, nabumetone, naproxen with esomeprazole, naproxen with esomeprazole strontium, once-daily naproxen (oral controlled release, pain), Alvogen, pelubiprofen, LY-210073, tenoxicam, licofelone, NS-398, bromfenac, L-746483, LY-255283, tenidap, tepoxalin, flobufen, ibuprofen, flurbiprofen, SKF-86002, SC-57666, WY-28342, CI-986, bermoprofen; Cytosolic phospholipase A2 inhibitors, such as AVX-002; Cytotoxic T-lymphocyte protein-4 modulators, such as belatacept, ES-210; Cytotoxic T-lymphocyte protein-4 stimulators, such as abatacept, abatacept biosimilar, BMS-188667; DHFR inhibitors, such as methotrexate, MPI-2505, MBP-Y003; Diamine acetyltransferase inhibitors, such as diminazene aceturate; Dihydroorotate dehydrogenase inhibitors, such as DHODH inhibitors (rheumatoid arthritis/autoimmune diseases), East China University of Science and Technology, ASLAN-003, laflunimus, leflunomide, HWA-486, ABR-224050; Elongation factor 2 inhibitors, such as denileukin diftitox; Eotaxin 2 ligand inhibitors, such as CM-102; EP4 prostanoid receptor antagonists, such as CR-6086; Erythropoietin receptor agonists, such as cibinetide; Fas ligands, such as AP-300; FGF-2 ligand inhibitors, such as RBM-007; FK506 binding protein-12 modulators, such as temsirolimus; Folate antagonists, such as methotrexate, MBP-Y003; Folate receptor agonists, such as folate receptor modulators (chimeric protein, cancer/rheumatoid arthritis), Proda Biotech; Folate receptor modulators, such as technetium (99mTc) etarfolatide; Fractalkine ligand inhibitors, such as E-6011; Fyn tyrosine kinase inhibitors, such as masitinib, laflunimus; G protein coupled receptor 15 antagonists, such as GPR15 antagonists (rheumatoid arthritis/HIV-mediated enteropathy), Omeros; GABA A receptor modulators, such as laflunimus; Glucocorticoid agonists, such as prednisolone, fosdagrocorat; Glucocorticoid antagonists, such as REC-200; Glucocorticoid induced leucine zipper stimulators, such as ART-G01; GM-CSF ligand inhibitors, such as namilumab, MORAb-022, lenzilumab; GM-CSF receptor antagonists, such as mavrilimumab; GM-CSF receptor modulators, such as GSK-3196165; Growth regulated protein alpha ligand inhibitors, such as T-5224; Hwith Kwith ATPase inhibitors, such as naproxen with esomeprazole, naproxen with esomeprazole strontium, ketoprofen with omeprazole, KEO-25001, HC-1004, PN-40020; Histamine H4 receptor antagonists, such as toreforant, GD-48; Histone deacetylase inhibitors, such as givinostat, CHR-5154; Histone deacetylase-6 inhibitors, such as CKD-506; HIV-1 gp120 protein inhibitors, such as maraviroc; HLA class II antigen DQ-2 alpha modulators, such as NexVax2; HLA class II antigen inhibitors, such as HLA-DR1/DR4 inhibitors (rheumatoid arthritis), Provid; HLA class II antigen modulators, such as ARG-301, recombinant T-cell receptor ligand (rheumatoid arthritis), Artielle; Hsp 70 family inhibitors, such as gusperimus trihydrochloride; Hypoxia inducible factor-1 inhibitors, such as 2-methoxyestradiol; IFNB gene stimulators, such as ART-102; I-kappa B kinase beta inhibitors, such as IMD-2560, IMD-0560; I-kappa B kinase inhibitors, such as bardoxolone methyl; IL-1 antagonists, such as rilonacept, IBPB-007-IL, antisense oligonucleotides (rheumatoid arthritis), Leiden University Medical Center, recombinant human interleukin-1 receptor antagonist (rheumatoid arthritis), Shanghai Fudan-Zhangjiang Bio-Pharmaceutical; IL-10 agonists, such as peg-ilodecakin; IL-11 agonists, such as oprelvekin; IL-12 antagonists, such as ustekinumab, briakinumab, ddRNAi therapy (rheumatoid arthritis), Medistem/Benitec; IL-15 antagonists, such as AMG-714, BNZ-132-2; IL-17 antagonists, such as ixekizumab, secukinumab, KD-025; IL-17 receptor modulators, such as CNTO-6785; IL-2 agonists, such as interleukin-2 follow-on biologic; IL-2 antagonists, such as IB-RA (injectable, rheumatoid arthritis), Innobioscience, IB-RA (oral, rheumatoid arthritis), Innobioscience, BNZ-132-2; IL-21 antagonists, such as NN-8828, BNZ-132-2; IL-23 antagonists, such as ustekinumab, briakinumab; IL-3 antagonists, such as anti-IL-3 mAbs (rheumatoid arthritis), University of Regensburg; IL-4 agonists, such as SER-130-AMI; IL-6 antagonists, such as olokizumab, clazakizumab, sirukumab, SA-237, tocilizumab, ALX-0061, FB-704A, OP—R003, peptide IL-6 antagonist, MEDI-5117, T-5224, humanized anti-IL-6 mAb, tocilizumab biosimilar, IL-6 neutralizing human antibodies, anti-IL6 antibody, RN-486, BLX-1002, AMG-220, FM-101, K-832, BLX-1025, esonarimod, TA-383; IL-6 receptor modulators, such as tocilizumab, tocilizumab biosimilar, RO-4877533; Immunoglobulin antagonists, such as iguratimod; Immunoglobulin G1 agonists, such as canakinumab, infliximab biobetter, infliximab biosimilar, BX-2922, STI-002, HF-1020; Immunoglobulin G1 antagonists, such as YHB-1411-2; Immunoglobulin G1 modulators, such as CFZ-533, lenzilumab; Immunoglobulin G2 antagonists, such as denosumab; Immunoglobulin G2 modulators, such as PF-547659; Immunoglobulin gamma Fc receptor II modulators, such as MGD-010; Immunoglobulin gamma Fc receptor IIB antagonists, such as XmAb-5871; Immunoglobulin kappa modulators, such as lenzilumab; Immunoglobulin M antagonists, such as IB-RA (injectable, rheumatoid arthritis), Innobioscience, IB-RA (oral, rheumatoid arthritis), Innobioscience; Inducible nitric oxide synthase inhibitors, such as SKLB-023; Inosine monophosphate dehydrogenase inhibitors, such as mycophenolate mofetil; Insulin sensitizers, such as rosiglitazone, THR-0921, HE-3286, BLX-1002; Integrin alpha-1/beta-1 antagonists, such as SAN-300; Integrin alpha-4/beta-1 antagonists, such as natalizumab; Integrin antagonists, such as PEG-HM-3, CY-9652; Interferon beta ligands, such as recombinant interferon beta-la, TA-383; Interferon gamma ligands, such as interferon gamma follow-on biologic; Interleukin 17A ligand inhibitors, such as ABT-122, bimekizumab, ABBV-257; Interleukin 17F ligand inhibitors, such as bimekizumab; Interleukin 23A inhibitors, such as guselkumab; Interleukin ligands, such as IBPB-007-IL; Interleukin receptor 17A antagonists, such as brodalumab; Interleukin-1 beta ligand inhibitors, such as canakinumab, rilonacept, T-5224, gevokizumab, BLX-1002, LY-2189102, PMI-001, K-832, CDP-484; Interleukin-10 ligands, such as PF-06687234; Interleukin-2 ligands, such as denileukin diftitox, recombinant interleukin-2, interleukin-2 follow-on biologic, recombinant human interleukin-2, interleukin-2 (injectable); Interleukin-4 ligands, such as Tetravil; Interleukin-6 ligand inhibitors, such as gerilimzumab, PF-4236921; Itk tyrosine kinase inhibitors, such as ARN-4079; JAK tyrosine kinase inhibitors, such as tofacitinib, SHR-0302, cerdulatinib, peficitinib, deuterated tofacitinib analog, SD-900, CVXL-0074; Jak1 tyrosine kinase inhibitors, such as ABT-494, baricitinib, ruxolitinib, filgotinib, tofacitinib, itacitinib, peficitinib, NIP-585, CS-944X, YJC-50018, GLPG-0555, MRK-12; Jak2 tyrosine kinase inhibitors, such as baricitinib, ruxolitinib, CT-1578; JAK3 gene inhibitors, such as GBL-5b; Jak3 tyrosine kinase inhibitors, such as decernotinib, tofacitinib, peficitinib, AC-0025, CS-944X, DNX-04042, MTF-003, ARN-4079, PS-020613; Jun N terminal kinase inhibitors, such as IQ-1S; KCNA voltage-gated potassium channel-3 modulators, such as MRAD-P1; Kelch like ECH associated protein 1 modulators, such as dimethyl fumarate; Kit tyrosine kinase inhibitors, such as imatinib, masitinib; LanC like protein 2 modulators, such as BT-11; LITAF gene inhibitors, such as GBL-5b; Lymphocyte function antigen-3 receptor antagonists, such as alefacept; Lyn tyrosine kinase inhibitors, such as masitinib; Macrophage mannose receptor 1 modulators, such as technetium Tc 99m tilmanocept; MAdCAM inhibitors, such as PF-547659; MAP kinase modulators, such as SKLB-023; MAP3K2 gene inhibitors, such as GBL-5b; MAPKAPK5 inhibitors, such as GLPG-0259; Matrix metalloprotease inhibitors, such as GLPG-0259; MCL1 gene inhibitors, such as seliciclib; MEK protein kinase inhibitors, such as binimetinib, AD-GL0001; MEK-1 protein kinase inhibitors, such as binimetinib; MEK-2 protein kinase inhibitors, such as binimetinib; Membrane copper amine oxidase inhibitors, such as BTT-1023, PRX-167700, vepalimomab; Metalloprotease-2 inhibitors, such as ERG-240; Metalloprotease-9 inhibitors, such as GS-5745, ERG-240; Midkine ligand inhibitors, such as CAB-102; Mitochondrial 10 kDa heat shock protein stimulators, such as INV-103; mTOR complex 1 inhibitors, such as everolimus; mTOR inhibitors, such as everolimus, temsirolimus; NAD ADP ribosyltransferase stimulators, such as denileukin diftitox; NAMPT gene inhibitors, such as ART-D01; NF kappa B inhibitor stimulators, such as denosumab; NFAT gene inhibitors, such as T-5224; NFE2L2 gene stimulators, such as bardoxolone methyl; Nicotinic acetylcholine receptor antagonists, such as RPI-78, RPI-MN; NK cell receptor modulators, such as masitinib; NKG2 A B activating NK receptor antagonists, such as monalizumab; NKG2 D activating NK receptor antagonists, such as NNC-0142-002; Nuclear erythroid 2-related factor 2 stimulators, such as dimethyl fumarate; Nuclear factor kappa B inhibitors, such as bardoxolone methyl, IB-RA (injectable, rheumatoid arthritis), Innobioscience, dehydroxymethylepoxyquinomicin, HE-3286, IMD-0560, MP-42, tarenflurbil, VGX-1027, SKLB-023, SP-650003, MG-132, SIM-916, VGX-350, VGX-300, GIT-027, SP-100030, MLN-1145, NVP—IKK-005; Nuclear factor kappa B modulators, such as REM-1086; Nuclear factor kappa B p105 inhibitors, such as REM-1086; Opioid growth factor receptor agonists, such as metenkefalin acetate with tridecactide acetate, FAR-404; Opioid receptor delta antagonists, such as HS-378; Osteoclast differentiation factor antagonists, such as denosumab, cyclic peptidomimetics (rheumatoid arthritis/osteoporosis), University of Michigan; Osteoclast differentiation factor ligand inhibitors, such as denosumab; Oxidoreductase inhibitors, such as etodolac, imidazole salicylate; P2X7 purinoceptor agonists, such as givinostat; p38 MAP kinase alpha inhibitors, such as VX-745, BMS-582949 prodrugs, BMS-751324; p38 MAP kinase inhibitors, such as BCT-197, losmapimod, ARRY-797; PDE 4 inhibitors, such as apremilast; PDE 5 inhibitors, such as PDE5 inhibitors (rheumatoid arthritis), University of Rochester; PDGF receptor agonists, such as oprelvekin; PDGF receptor antagonists, such as imatinib, masitinib; PDGF-B ligand inhibitors, such as SL-1026; PERK gene inhibitors, such as binimetinib; Phosphoinositide-3 kinase delta inhibitors, such as duvelisib, RP-6503, CT-732, INK-007, GNE-293; Phosphoinositide-3 kinase gamma inhibitors, such as duvelisib, RP-6503; Phospholipase A2 inhibitors, such as AVX-002, human secreted phospholipase A2 type IIA-integrin binding inhibiting peptides (rheumatoid arthritis/asthma/Alzheimer's disease/cancer), University of California, Davis, AK-106, varespladib methyl, Ro-31-4493, BM-162353, Ro-23-9358, YM-26734; Platelet activating factor receptor antagonists, such as piperidone hydrochloridum; PPAR gamma agonists, such as rosiglitazone, THR-0921, rosiglitazone XR, etalocib; Programmed cell death protein 1 modulators, such as INSIX RA; Prostaglandin D synthase stimulators, such as HF-0220; Protein arginine deiminase inhibitors, such as PAD inhibitors (rheumatoid arthritis), Leiden University Medical Center/LURIS; Protein tyrosine kinase inhibitors, such as leflunomide; PurH purine biosynthesis protein inhibitors, such as mycophenolate mofetil; Rho associated protein kinase 2 inhibitors, such as KD-025; Seprase inhibitors, such as anti-fibroblast-activation protein (FAP) antibody radiotracers (rheumatoid arthritis), Hoffmann-La Roche/Radboud University; Signal transducer CD24 modulators, such as CD24-IgFc; Signal transduction inhibitors, such as imatinib; Sodium glucose transporter-2 inhibitors, such as THR-0921; Sphingosine 1 phosphate phosphatase modulators, such as S1p modulators (oral, multiple sclerosis/ulcerative colitis/rheumatoid arthritis), Akaal Pharma; STAT3 gene inhibitors, such as bardoxolone methyl, vidofludimus; Superoxide dismutase stimulators, such as imisopasem manganese; SYK family tyrosine kinase inhibitors, such as MK-8457; Syk tyrosine kinase inhibitors, such as fostamatinib, entospletinib, KDDF-201110-06, HMPL-523, cerdulatinib, AB-8779, GS-9876, PRT-2607, CVXL-0074, CG-103065 and CG-026806; Syndecan-1 inhibitors, such as indatuximab ravtansine; T cell receptor antagonists, such as TCR inhibiting SCHOOL peptides (systemic/topical, rheumatoid arthritis/dermatitis/scleroderma), SignaBlok, CII modified peptide (rheumatoid arthritis), Peking University; T cell receptor modulators, such as ARG-301; T cell surface glycoprotein CD28 inhibitors, such as abatacept, belatacept, abatacept biosimilar, RhuDex, BMS-188667; T cell surface glycoprotein CD28 stimulators, such as TAB-08; TAK1 binding protein modulators, such as epigallocatechin 3-gallate; Talin modulators, such as short-form talin regulators (rheumatoid arthritis), KayteeBio; T-cell differentiation antigen CD6 inhibitors, such as itolizumab; T-cell surface glycoprotein CD8 inhibitors, such as tregalizumab; Tenascin modulators, such as Tetravil; TGF beta agonists, such as tregalizumab; Thymulin agonists, such as Syn-1002; TLR-2 antagonists, such as VB-201, P-13; TLR-4 antagonists, such as VB-201, P-13; TLR-9 antagonists, such as P-13; TNF alpha ligand inhibitors, such as adalimumab biosimilarYHB-1411-2, adalimumab, infliximab, infliximab biosimilar, recombinant humanized anti-TNF-alpha monoclonal antibody, certolizumab pegol, golimumab, ozoralizumab, AT-132, etanercept biosimilar, ISIS-104838, ISU-202, CT-P17, MB-612, Debio-0512, anti-TNF alpha human monoclonal antibody, infliximab biobetter, UB-721, KN-002, DA-3113, BX-2922, R-TPR-015, BOW-050, PF-06410293, CKD-760, CHS-1420, GS-071, ABP-710, STI-002, BOW-015, FKB-327, BAX-2200, HLX-03, BI-695501, CNTO-148, MYL-1401AABP-501, HOT-3010, BAX-2923, SCH-215596, ABT-D2E7, BAT-1406, Xpro-1595, Atsttrin, SSS-07, golimumab biosimilar, TA-101, adalimumab follow-on biologic, BLX-1002, ABX-0401, TAQ-588, golimumab biosimilar, TeHL-1, placulumab, PMI-001, tgAAV-TNFR:Fc, K-832, CYT-007-TNFQb, SSR-150106, PassTNF, Verigen, DOM-0200, DOM-0215, AME-527, anti-TNF-alpha mAb, GENZ-38167, BLX-1028, CYT-020-TNFQb, CC-1080, CC-1069; TNF alpha ligand modulators, such as MM-A01-01, CDP-571, camobucol; TNF antagonists, such as etanercept, certolizumab pegol, etanercept follow-on biologic, etanercept biosimilar, DNX-114, TNF antagonist with IL-12 antagonist (rheumatoid arthritis), University of Oxford, BN-006, SCB-131, pegsunercept, GBL-5b, ACE-772, onercept, DE-096, PN-0615, lenercept, ITF-1779, MDL-201112, BAX-2200, SCB-808, DA-3853, HD-203; TNF gene inhibitors, such as GIBH-R-001-2; TNF receptor modulators, such as recombinant TNF receptor 2-Fc fusion protein mutant, T-0001, tgAAV-TNFR:Fc; TNFSF11 gene inhibitors, such as denosumab; Transcription factor p65 inhibitors, such as REM-1086; Transcription factor RelB inhibitors, such as REM-1086; Transferrin modulators, such as methotrexate, MBP-Y003; Tumor necrosis factor 13C receptor antagonists, such as VAY-736; Tumor necrosis factor 15 ligand inhibitors, such as anti-TL1A antibodies (rheumatoid arthritis/inflammatory bowel disease), NIAMS; Tumor necrosis factor ligand 13 inhibitors, such as atacicept; Tumor necrosis factor ligand inhibitors, such as ABBV-257, etanercept biosimilar, ABT-122; Type I IL-1 receptor antagonists, such as anakinra, anakinra biosimilar, anakinra follow-on biologic, AXXO; Type I TNF receptor antagonists, such as NM-9405; Type II TNF receptor modulators, such as etanercept, SCB-131, etanercept biosimilar, etanercept follow-on biologic, BAX-2200, SCB-808, LBEC-0101, DMB-3853, DWP-422, BT-D001, DA-3853; Unspecified GPCR agonists, such as NCP-70X; VEGF receptor antagonists, such as 2-methoxyestradiol and NSC-650853, SL-1026; VEGF-2 receptor antagonists, such as CG-026806; VEGF-2 receptor modulators, such as VEGFR2 neutralizing antibody (rheumatoid arthritis), University of Rochester; VEGF-B ligand inhibitors, such as CSL-346; X-linked inhibitor of apoptosis protein inhibitors, such as IAP inhibitors (oral), Pharmascience; and Zap70 tyrosine kinase inhibitors, such as MK-8457, CT-5332.

### Combinations for Metabolic Diseases or Conditions

Examples of metabolic disorders include, without limitation, diabetes, including type I and type II diabetes, metabolic syndrome, dyslipidemia, obesity, glucose intolerance, hypertension, elevated serum cholesterol, and elevated triglycerides. Examples of therapeutic agents used to treat metabolic disorders include antihypertensive agents and lipid lowering agents. Additional therapeutic agents used to treat metabolic disorders include insulin, sulfonylureas peroxisome proliferator activated receptor gamma (PPAR-γ) agonists, such as thiazolidinediones such as Pioglitazones, biguanides, alpha-glucosidase inhibitors, Vitamin E and incretin mimetics. Thus, one aspect of the disclosure is a method of treating a metabolic disease comprising administering a chemical entity of the disclosure in combination with one or more compounds useful for the treatment of metabolic diseases to a subject, particularly a human subject, in need thereof.

### EXAMPLES

The following examples are included to demonstrate specific embodiments of the disclosure. It should be appreciated by those of skill in the art that the techniques disclosed in the examples which follow represent techniques to function well in the practice of the disclosure, and thus can be considered to constitute specific modes for its practice. However, those of skill in the art should, in light of the present disclosure, appreciate that many changes can be made in the specific embodiments which are disclosed and still obtain a like or similar result without departing from the spirit and scope of the disclosure.

### Instruments and Methods

### X-Ray Powder Diffraction (XRPD)

X-ray powder diffraction (XRPD) analysis was conducted on a diffractometer (PANalytical XPERT-PRO, PANalytical B. V., Almelo, Netherlands) using copper radiation (Cu Kα, λ=1.541874). Samples were spread evenly on a zero background sample plate. The generator was operated at a voltage of 45 kV and amperage of 40 mA. Slits were Soller 0.02 rad, antiscatter 1.0°, and divergence. Scans were performed from 2 to 40°2θ with a 0.0167 step size. Data analysis was performed using X′Pert Viewer V 1.2d (PANalytical B.V., Almelo, Netherlands). X-ray powder diffraction analysis was also conducted on a diffractometer (Rigaku MiniFlex, Rigaku Corporation, Tokyo, Japan) using copper radiation (Cu Kα, λ=1.541874). Samples were spread evenly on a zero background sample plate. The generator was operated at a voltage of 40 kV and amperage of 15 mA. Scans were performed from 2 to 40°2°θ with a 0.050 degree step size and a speed of 2-8 degrees/minute. Data analysis was also performed using X′Pert Data Viewer V 1.2d (PANalytical B.V., Almelo, Netherlands)

### Differential Scanning Calorimetry (DSC)

Differential Scanning Calorimetry (DSC) was run on a Q2000 (TA Instruments, New Castle, DE) by loading 1-5 mg of material into a crimped or open Tzero standard aluminum pan and heating the sample at 10° C./min from 20 to 300° C. or above. The sample and reference pans were under a 50 mL/min nitrogen purge. Data analysis was completed using Universal Analysis 2000 Version 4.5A (TA Instruments, New Castle, DE).

### Thermogravimetric Analysis (TGA)

Thermogravimetric analysis (TGA) was used to evaluate sample weight loss as a function of temperature on either a Q5000 or Q500 (TA Instruments, New Castle, DE), by loading 1-10 mg of material onto a weigh pan and heating the sample to 350° C. or above at a rate of 10° C./min. The sample and reference pans were under a 60 mL/min and 40 mL/min nitrogen purge, respectively. Data analysis was completed using Universal Analysis 2000 Version 4.5A (TA Instruments, New Castle, DE).

Thermogravimetric-Mass Spectrometer analysis (TG-MS) was used to identify volatile off-gassing and to evaluate sample weight loss as a function of temperature on a Discovery TGA (TA Instruments, New Castle, DE) by loading 1-10 mg of material onto a weigh pan and heating the sample to fully desolvate the material at a rate of 20° C./min. The sample and reference pans were under a 60 mL/min and 40 mL/min nitrogen purge, respectively. Data analysis was completed using TRIOS (TA Instruments, New Castle, DE). The mass spectrometer is a Discovery MS (TA Instruments, New Castle, DE) benchtop quadrupole instrument.

### Dynamic Vapor Sorption (DVS)

Hygroscopicity was studied using dynamic vapor sorption (DVS, TA Q5000 SA, TA Instruments, New Castle, DE or DVS, DVS Intrinsic, Surface Measurement Systems, London, UK). A sample (2-20 mg) was placed in an aluminum DVS pan and loaded on the sample side of the twin pan balance. The water sorption and desorption were studied as a function of relative humidity (RH) at 25° C. In 10% RH increments, the relative humidity was increased from 5% RH to 95% RH and then decreased back to 5%. Each relative humidity increment had an equilibration time of 180 minutes, unless weight change % was less than 0.002% in 30 minutes. Data analysis was performed using Universal Analysis 2000 Version 4.7A (TA Instruments, New Castle, DE) for TA DVS runs and Microsoft Excel for SMS DVS runs.

### Proton Nuclear Magnetic Resonance (¹H NMR)

Proton Nuclear Magnetic Resonance (¹H NMR) spectra were collected on a Varian 400-MR 400 MHz instrument with 7620AS sample changer. The default proton parameters are as follows: spectral width: 14 to −2 ppm (6397.4 Hz); relaxation delay: 1 sec; pulse: 45 degrees; acquisition time: 2.049 sec; number of scans or repetitions: 8; temperature: 25° C. Samples were prepared in dimethyl sulfoxide-d₆, unless otherwise stated. Off-line analysis was carried out using Mnova software.

### List of Abbreviations and Acronyms

Abbreviation Meaning
° C. degrees Celsius
ACN acetonitrile
ESA ethylsulfonic acid
EtOH ethanol
HCl hydrochloric acid
IPA isopropanol
K₃PO₄ tripotassium phosphate
MTBE methyl tert-butyl ether
MIBK methyl isobutyl ketone
2-MeTHF 2-methyl tetrahydrofuran
RH relative humidity
THF tetrahydrofuran
X-Phos dicyclohexyl[2′,4′,6′-tris(propan-2-yl)[1,1′-biphenyl]-2-yl]phosphane

### Example 1. Compound I Form I

### Coupling Reaction of 4-(((R)-1-amino-1-oxopropan-2-yl)amino)-6-chloro-N—((R)-2-fluoro-3-hydroxy-3-methylbutyl)nicotinamide with 7-(4,4,5,5-tetramethyl-1,3,2-dioxaborolan-2-yl)pyrrolo[1,2-b]pyridazine-3-carbonitrile to 4-(((R)-1-amino-1-oxopropan-2-yl)amino)-6-(3-cyanopyrrolo[1,2-b]pyridazin-7-yl)-N—((R)-2-fluoro-3-hydroxy-3-methylbutyl)nicotinamide

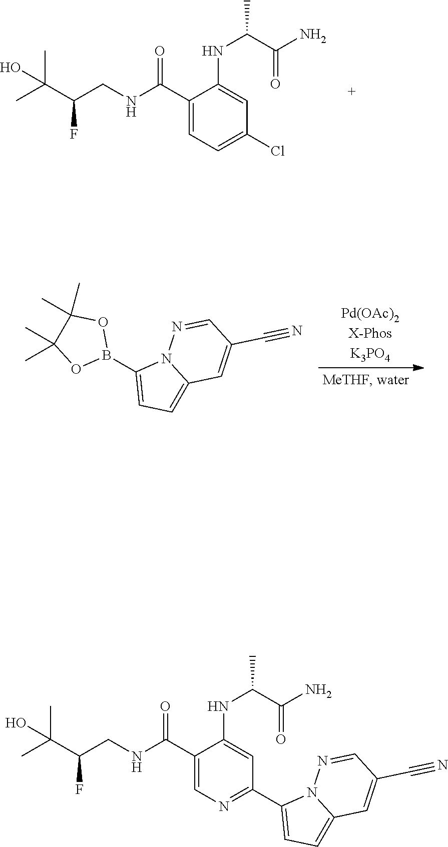 [SMILES: C[C@@H](Nc1cc(Cl)ccc1C(=O)NC[C@@H](F)C(C)(C)O)C(N)=O ; CC1(C)OB(c2ccc3cc(C#N)cnn23)OC1(C)C ; C[C@@H](Nc1cc(-c2ccc3cc(C#N)cnn23)ncc1C(=O)NC[C@@H](F)C(C)(C)O)C(N)=O]

A reactor was charged with 4-(((R)-1-amino-1-oxopropan-2-yl)amino)-6-chloro-N—((R)-2-fluoro-3-hydroxy-3-methylbutyl)nicotinamide (1.0 equiv, scaling factor), 7-(4,4,5,5-tetramethyl-1,3,2-dioxaborolan-2-yl)pyrrolo[1,2-b]pyridazine-3-carbonitrile (1.6 equiv), XPhos (0.05 equiv), palladium acetate (0.025 equiv) and 2-methyltetrahydrofuran (17.0 volumes). The resulting mixture was agitated and a solution of potassium phosphate tribasic (2.0 equiv) in water (2.5 volumes) was added to the mixture. The reaction mixture was agitated and heated to about 75° C. for about 4 hours, then cooled to about 40° C. The resulting slurry was filtered. The cake was washed with toluene (9.0 volumes), acetonitrile (5.0 volumes) and aqueous citric acid solution (10% w/w), and dried at about 40° C. The solids were charged to a reactor and slurried in N-methyl-2-pyrrolidinone (5.0 volumes) at about 40° C. The slurry was filtered and washed with N-methyl-2-pyrrolidinone (2.0 volumes) and the resulting organic stream was concentrated to about 5 volumes. The mixture was charged to a reactor, followed by diatomaceous earth (200% w/w), trithiocyanuric acid trisodium hydrate (0.3 equiv), and Darco G-60 (50% w/w) and mixed at about 40° C. for about 2 h. The mixture was filtered and the filtrate concentrated to about 1 volume. Ethanol (18.0 volumes) was added at about 40° C. and aged at about 50° C. for about 2 h. Water (18.0 volumes) was added at about 35° C. and aged for about 2 h and cooled to about 20° C. over about 3 h. The slurry was filtered and washed with water (5.0 volumes). The reactor contents were filtered and the cake dried for approximately 21 hours at approximately 40° C. to give 4-(((R)-1-amino-1-oxopropan-2-yl)amino)-6-(3-cyanopyrrolo[1,2-b]pyridazin-7-yl)-N—((R)-2-fluoro-3-hydroxy-3-methylbutyl)nicotinamide.

1H NMR (400 MHz, Methanol-d₄) δ 8.76 (d, J=2.2 Hz, 1H), 8.69-8.63 (m, 2H), 8.05 (d, J=4.8 Hz, 1H), 7.88 (s, 1H), 7.22 (d, J=5.1 Hz, 1H), 4.91 (t, J=7.6 Hz, 2H), 4.73 (dd, J=8.2, 4.4 Hz, 2H), 4.52-4.28 (m, 3H), 3.80-3.40 (m, 5H), 2.76 (dd, J=13.9, 7.0 Hz, 2H), 2.24 (s, 2H), 1.29 (d, J=1.7 Hz, 6H).

### Amide Dehydration Reaction of 4-(((R)-1-amino-1-oxopropan-2-yl)amino)-6-(3-cyanopyrrolo[1,2-b]pyridazin-7-yl)-N—((R)-2-fluoro-3-hydroxy-3-methylbutyl)nicotinamide to 4-(((R)-1-cyanoethyl)amino)-6-(3-cyanopyrrolo[1,2-b]pyridazin-7-yl)-N—((R)-2-fluoro-3-hydroxy-3-methylbutyl)nicotinamide (Compound I)

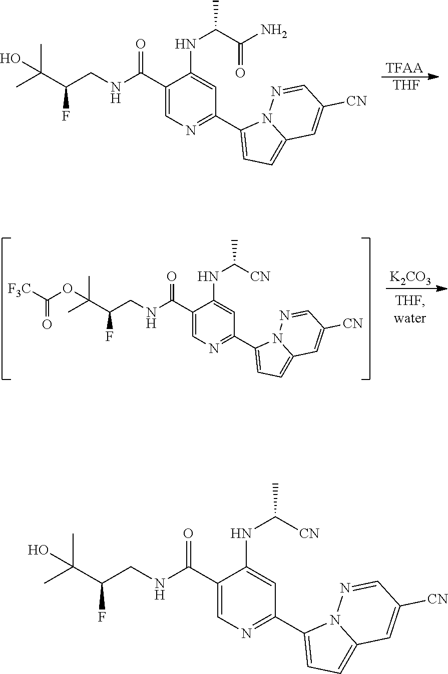

A reactor was charged with 4-(((R)-1-amino-1-oxopropan-2-yl)amino)-6-(3-cyanopyrrolo[1,2-b]pyridazin-7-yl)-N—((R)-2-fluoro-3-hydroxy-3-methylbutyl)nicotinamide and THF (17.0 volumes). Pyridine (5.0 equiv) was charged and the mixture was cooled to about 0° C. Trifluoroacetic anhydride (2.9 equiv) was charged and resulting mixture was agitated at about 0° C. for about 1 hour. A sodium carbonate solution (1M, 15 volumes) was charged over about 1 h and the mixture warmed to about 20° C. over about 1 h. Water (20.0 volumes) and ethyl acetate (20.0 volumes) were charged and the mixture filtered over celite and rinsed with ethyl acetate (20.0 volumes). The layers were separated and the aqueous stream extracted with 2-methyltetrahydrofuran (20.0 volumes). The organic streams were combined and washed with a saturated aqueous ammonium chloride solution (20.0 volumes). The resulting organic stream was solvent swapped into ethanol (15.0 volumes) and n-heptane (50.0 volumes) was added over about 1 h at about 20° C. After aging the slurry for about 3 hours, the slurry was filtered, and the cake was washed with n-heptane (2.0 volumes). The resulting cake was dried at about 50° C. to provide 4-(((R)-1-cyanoethyl)amino)-6-(3-cyanopyrrolo[1,2-b]pyridazin-7-yl)-N—((R)-2-fluoro-3-hydroxy-3-methylbutyl)nicotinamide (Compound I).

¹H NMR (400 MHz, DMSO-d₆): δ 8.89 (t, J=5.8 Hz, 1H), 8.81 (s, 1H), 8.78 (d, J=7.2 Hz, 1H), 8.61 (d, J=2.4 Hz, 1H), 8.22 (s, 1H), 7.85 (d, J=4.4 Hz, 1H), 7.10 (d, J=4.8 Hz, 1H), 4.86 (p, J=6.9 Hz, 1H), 4.40 (ddd, J=49.2, 9.2, 2.0 Hz, 1H), 3.75 (dddd, J=37.2, 14.4, 5.2, 2.0 Hz, 1H), 3.43 (m, 1H), 1.72 (d, J=7.2 Hz, 3H), 1.19 (d, J=1.6 Hz, 3H), 1.18 (d, J=1.2 Hz, 3H) ppm.

¹³C NMR (100 MHz, DMSO-d₆): δ 167.5, 152.0, 149.7, 149.2, 141.7, 133.8, 130.6, 127.3, 119.7, 118.5, 117.2, 110.1, 106.9, 104.0, 96.9 (d, J=177.8 Hz), 93.5, 69.5 (d, J=19.9 Hz), 39.4, (m), 26.1 (d, J=3.8 Hz), 24.7 (d, J=3.8 Hz), 18.5 ppm.

Compound I Form I is a crystalline unsolvated form of Compound I free base. Form I was prepared by crystallizing Compound I free base as prepared above from pure ethyl acetate upon concentrating to dryness.

The XRPD pattern for Form I is shown in FIG. 1. The DSC curve of Form I is shown in FIG. 2. The DSC data shows an endothermic event with an onset temperature of about 155° C., followed by another endothermic event with an onset temperature of about 175° C. The TGA of Form I is shown in FIG. 3. The minimal weight loss (about 1 wt %) observed in the TGA data from ambient temperature to about 155° C. indicates that Form I is unsolvated.

### Example 2. Compound I Form II

Compound I Form II is a crystalline unsolvated form of Compound I free base. Form II was prepared by charging Compound I Form I to ˜1 mL of pure ethyl acetate until a slurry is formed and stirred at room temperature for ˜7 days. The solids were isolated using a NANOSEP centrifugal filter.

The XRPD pattern for Form II is shown in FIG. 4. The DSC curve of Form II is shown in FIG. 5. The DSC data shows an endothermic event with an onset temperature of about 191° C. attributed to the melt of Form II. The TGA of Form II is shown in FIG. 6. The minimal weight loss (about 1 wt %) observed in TGA data from ambient temperature to about 200° C. indicates that Form II is unsolvated.

### Example 3. Compound I Form III

Compound I Form III is a crystalline unsolvated form of Compound I free base. Form III was prepared by heating Compound I Form V to about 110° C. in a variable temperature XRPD.

The XRPD pattern for Form III is shown in FIG. 7.

### Example 4. Compound I Form IV

Compound I Form IV is a crystalline unsolvated form of Compound I free base. Form IV was prepared by charging 6.69 g of Compound I into ˜75 mL of pure ethanol at ˜40° C. to 50° C. and seeding with Compound I Form II. The slurry was held overnight and then cooled to ˜10° C. before the solids were isolated using a disposable fritted funnel.

The XRPD pattern for Form IV is shown in FIG. 8. The DSC curve of Form IV is shown in FIG. 9. The DSC data shows an endothermic event with an onset temperature of about 192° C. attributed to the melt of Form IV. The TGA of Form IV is shown in FIG. 10. The minimal weight loss (<1 wt %) observed in TGA data indicates that the sample is unsolvated. The DVS of Form IV is shown in FIG. 11. DVS analysis shows that Form IV is non-hygroscopic, absorbing less than 0.1 wt % water at 25° C. and 95% RH. XRPD analysis of the sample post DVS analysis showed no form change.

### Example 5. Compound I Form V

Compound I Form V is a crystalline monohydrate form of Compound I free base. Form V was prepared by charging ˜2 g of Compound I Form VI to ˜10 mL of pure ethyl acetate and stirred at room temperature overnight. The solids were isolated using a Buchner funnel and filter paper.

The XRPD pattern for Form V is shown in FIG. 12. The DSC curve of Form V is shown in FIG. 13. The endothermic event with an onset temperature of about 71° C. is attributed to the dehydration of Form V and conversion to Form III. The endothermic event with an onset temperature of about 152° C. is attributed to the melt of Form III. The exothermic event with an onset temperature of about 171° C. and the endothermic event with an onset temperature of about 189° C. are attributed to the crystallization and melt of Form II or IV, respectively. The TGA of Form V is shown in FIG. 14. Based on the TGA weight loss up to about 100° C., the amount of water correlates to about 1 mole equivalent (4 wt % theoretical) of water. The DVS of Form V is shown in FIG. 15.

### Example 6. Compound I Form VI

Compound I Form VI is a semi-crystalline hydrated form of Compound I free base. Form VI was prepared by charging 0.3 g of Compound I free base to 45 mL of water and stirring at room temperature for ˜6 days. The solids were isolated using a Buchner funnel and filter paper.

The XRPD pattern for Form VI is shown in FIG. 16. The DSC curve of Form VI is shown in FIG. 17. The endothermic event with an onset temperature of about 47° C. is attributed to the dehydration of Form VI. The endothermic event with an onset temperature of about 111° C. and about 123° C. were not investigated. The TGA of Form VI is shown in FIG. 18. Based on the TGA weight loss from ambient to about 140° C., the amount of water correlates to about 1.25 mole equivalents (about 5.2 wt % theoretical) of water. The DVS of Form VI is shown in FIG. 19.

### Example 7. Compound I Form VII

Compound I Form VII is a crystalline water:THF solvated form of Compound I free base. Form VII was prepared by dissolving approximately 2 g of Compound I free base in about 40 mL of THF at room temperature then rotovapped to dryness.

The XRPD pattern for Form VII is shown in FIG. 20. The DSC curve of Form VII is shown in FIG. 21. The endothermic event with onset temperatures of about 17° C. and about 54° C. are attributed to the desolvation of Form VII. The endothermic events with onset temperature of about 138° C. and about 187° C. were not investigated. The TGA of Form VII is shown in FIG. 22. The TGA data shows a weight loss of about 7.9 wt. % from ambient to about 160° C., indicating that Form VII is solvated, which was later confirmed by TGA-MS analysis to be the loss of water and THF, respectively. The DVS of Form VII is shown in FIG. 23.

### Example 8. Compound I Form VIII

Compound I Form VIII is a crystalline water:ACN solvated form of Compound I free base. Form VIII was prepared by slurrying Compound I Form VII in ACN at room temperature. The solids were isolated using a Buchner funnel and filter paper.

The XRPD pattern for Form VIII is shown in FIG. 24. The DSC curve of Form VIII is shown in FIG. 25. The endothermic events with onset temperatures of about 17° C. and about 99° C. are attributed to the desolvation of Form VIII. The TGA of Form VIII is shown in FIG. 26. The TGA data shows a weight loss of about 1.4 wt. % from ambient to about 52° C. and another weight loss of about 6.6 wt % from about 52° C. to about 160° C., indicating that Form VIII is solvated, which was later confirmed by TGA-MS analysis to be the loss of water and THF, respectively. The DVS of Form VIII is shown in FIG. 27.

### Example 9. Compound I Form IX

Compound I Form IX is a crystalline water:2-MeTHF solvated form of Compound I free base. Form IX was prepared by slurrying Compound I Form VII in 2-MeTHF at room temperature. The solids were isolated using a Buchner funnel and filter paper.

The XRPD pattern for Form IX is shown in FIG. 28. The DSC curve of Form IX is shown in FIG. 29. The endothermic events with onset temperatures of about 20° C. and about 79° C. are attributed to the desolvation of Form IX. The TGA of Form IX is shown in FIG. 30. The TGA data shows a weight loss of about 2.7 wt. % from ambient to about 50° C. and another weight loss of about 15.5 wt % from about 50° C. to about 114° C., indicating that Form IX is solvated, which was later confirmed by TGA-MS analysis to be the loss of water and 2-MeTHF, respectively. The DVS of Form IX is shown in FIG. 31.

### Example 10. Compound I Form X

Compound I Form X is likely a crystalline isostructural solvated form of Compound I free base. Form X was prepared by charging Compound I free base to −1 mL of IPA until a slurry is formed and stirred it at room temperature for about 7 days. Form X was also prepared in a similar way using acetone or MTBE. The solids were isolated using a NANOSEP centrifugal filter.

The XRPD pattern for Form X is shown in FIG. 32. The DSC curve of Form X is shown in FIG. 33. The endothermic event with onset temperature of about 43° C. is attributed to the desolvation of Form X. The TGA of Form X is shown in FIG. 34. The TGA data shows a weight loss of about 5.1 wt % from ambient to about 150° C. indicating that Form X is solvated. A sample of Form X was heated in the TGA to about 150° C. and was subsequently analyzed by XRPD. The results showed that upon desolvation, Form X converts to Form XIII.

### Example 11. Compound I Form XI

Compound I Form XI is likely a crystalline isostructural solvated form of Compound I free base. Form XI was prepared by charging Compound I free base to ˜1 mL of MIBK until a slurry is formed and stirred at room temperature for about 7 days. Form XI was also prepared in a similar way using THF. The solids were isolated using a NANOSEP centrifugal filter.

The XRPD pattern for Form XI is shown in FIG. 35.

### Example 12. Compound I Form XII

Compound I Form XII is a crystalline form of Compound I free base. Form XII was prepared as a mixture with Compound I Form V by charging Compound I free base to ˜1 mL of toluene until a slurry is formed and stirred at room temperature for about 7 days. Form XII was also prepared in a similar way using heptane. The solids were isolated using a NANOSEP centrifugal filter.

The XRPD pattern for Compound I Form XII is shown in FIG. 36.

### Example 13. Compound I Form XIII

Compound I Form XIII is a crystalline unsolvated form of Compound I free base. Form I was prepared by desolvating Compound I Form X in a TGA at 150° C.

The XRPD pattern for Compound I Form XIII is shown in FIG. 37.

### Example 14. Compound I Amorphous Form

Compound I amorphous Form is a non-crystalline unsolvated form of Compound I free base. The amorphous Form was prepared by slurrying approximately 0.3 g of semi-crystalline Compound I free base in 10 mL of 2-MeTHF at room temperature overnight. Once the solids converted to Compound I Form IX, the solids were filtered and dried in a vacuum oven at 100° C. overnight.

The XRPD pattern for the amorphous Form is shown in FIG. 38.

### Example 15. Compound I Mono-Citrate Form I

Compound I mono-citrate Form I is a crystalline unsolvated mono-citrate salt of Compound I. Mono-citrate Form I was prepared by charging 1 mL of EtOH containing 1.5 molar equivalents of citric acid to a slurry of approximately 0.2 g of Compound I free base in 3 mL of EtOH at room temperature and stirring overnight.

The XRPD pattern for mono-citrate Form I is shown in FIG. 39. The DSC curve of mono-citrate Form I is shown in FIG. 40. The DSC data shows 2 endothermic events with onset temperatures of about 145° C. and about 187° C. attributed to the conversion to Compound I mono-citrate Form II and the loss of citric acid, respectively. The TGA of mono-citrate Form I is shown in FIG. 41. The minimal weight loss (about 0.6 wt %) observed in the TGA data from ambient temperature to about 150° C. indicates that mono-citrate Form I is unsolvated. The DVS of mono-citrate Form I is shown in FIG. 42. The ¹H NMR of mono-citrate Form I is shown in FIG. 43. NMR analysis shows that Compound I mono-citrate Form I contains about 1 mole equivalent of citric acid.

### Example 16. Compound I Mono-Citrate Form II

Compound I mono-citrate Form II is a crystalline unsolvated mono-citrate salt of Compound I. Mono-citrate Form II was prepared by heating Compound I mono-citrate Form I in a variable temperature XRPD at 170° C.

The XRPD pattern for mono-citrate Form II is shown in FIG. 44.

### Example 17. Compound I Mono-Citrate Form III

Compound I mono-citrate Form III is a crystalline monohydrate form of mono-citrate salt of Compound I. Mono-citrate Form III was prepared by slurrying approximately 1 g of Compound I Form II with 1.5 molar equivalents of citric acid in 20 mL of EtOH at 20° C. overnight.

The XRPD pattern for mono-citrate Form III is shown in FIG. 45. The DSC curve of mono-citrate Form III is shown in FIG. 46. The endothermic events with onset temperatures of about 25° C. and about 159° C. are attributed to the loss of water and decomposition of citric acid, respectively. The TGA of mono-citrate Form III is shown in FIG. 47. Based on the TGA weight loss, the amount of water correlates to about 1.5 mole equivalents (4.1 wt. % theoretical) of water. The DVS of mono-citrate Form III is shown in FIG. 48.

### Example 18. Compound I Mono-Citrate Form IV

Compound I mono-citrate Form IV is likely a crystalline mono-hexafluoro-2-propanol solvated form of mono-citrate salt of Compound I. Mono-citrate Form VI was prepared by dissolving Compound I mono-citrate in 20 mL of hexafluoro-2-propanol at room temperature and then using a rotavapor to remove the hexafluoro-2-propanol. The resulting solids were then isolated and analyzed by XRPD.

The XRPD pattern for mono-citrate Form IV is shown in FIG. 49. The DSC curve of mono-citrate Form IV is shown in FIG. 50. The endothermic events with onset temperatures of about 75° C. and about 183° C. are attributed to the loss of hexafluoro-2-propanol and decomposition of citric acid, respectively. The endothermic event with an onset temperature of about 139° C. was not investigated. The TGA of mono-citrate Form IV is shown in FIG. 51. The weight loss (about 22.5 wt %) observed in the TGA data from ambient temperature to about 140° C. indicates that Form mono-citrate IV is solvated, and was later confirmed by TGA-MS analysis to be hexafluoro-2-propanol. The DVS of mono-citrate Form IV is shown in FIG. 52.

### Example 19. Compound I Hemi-Citrate Form I

Compound I hemi-citrate Form I is a crystalline monohydrate hemi-citrate salt of Compound I. Hemi-citrate Form I was prepared by charging Compound I mono-citrate Form I to ˜1 mL of EtOH/water (73.7:26.3 v/v) until a slurry is formed and stirred at room temperature for about ˜6 days. The solids were isolated using a NANOSEP centrifugal filter.

The XRPD pattern for hemi-citrate Form I is shown in FIG. 53. The DSC curve of hemi-citrate Form I is shown in FIG. 54. The endothermic events with onset temperatures of about 70° C. and about 153° C. are attributed to the loss of water and decomposition of citric acid, respectively. The endothermic event with an onset temperature of about 133° C. was not investigated. The TGA of hemi-citrate Form I is shown in FIG. 55. The weight loss (about 2.8 wt %) observed in the TGA data from ambient temperature to about 100° C. indicates that hemi-citrate Form I is solvated, and was later confirmed by TGA-MS analysis to be water. Based on the TGA weight loss, the amount of water correlates to about 1 mole equivalent (3.3 wt % theoretical) of water. The weight loss (about 15 wt %) observed in the TGA data from about 140° C. to about 200° C. was confirmed by TGA-MS analysis to be the decomposition of citric acid, which produces water and carbon dioxide. The DVS of hemi-citrate Form I is shown in FIG. 56. The ¹H NMR of hemi-citrate Form I is shown in FIG. 57.

### Example 20. Compound I Mono-HCl Form I

Compound I mono-HCl Form I is a crystalline unsolvated mono-hydrochloride salt of Compound I. Mono-HCl Form I was prepared by slurrying approximately 0.2 g of Compound I free base in 3 mL of EtOH and charging 1 mL of EtOH containing approximately 1.5 molar equivalents of concentrated hydrochloric acid at room temperature. The solids were isolated using a NANOSEP centrifugal filter.

The XRPD pattern for mono-HCl Form I is shown in FIG. 58. The DSC curve of mono-HCl Form I is shown in FIG. 59. The TGA of mono-HCl Form I is shown in FIG. 60. The minimal weight loss (about 0.2 wt %) observed in the TGA data from ambient temperature to about 100° C. indicates that mono-HCl Form I is unsolvated. The DVS of mono-HCl Form I is shown in FIG. 61.

### Example 21. Compound I Mono-Maleate Form I

Compound I mono-maleate Form I is a crystalline unsolvated mono-maleate salt of Compound I. Mono-maleate Form I was prepared by slurrying approximately 0.2 g of Compound I free base in 3 mL of EtOH and charging 1 mL of EtOH containing approximately 1.5 molar equivalents of maleic acid at room temperature. The solids were isolated using a NANOSEP centrifugal filter.

The XRPD pattern for mono-maleate Form I is shown in FIG. 62. The DSC curve of mono-maleate Form I is shown in FIG. 63. The DSC data shows an endothermic event with an onset temperature of about 188° C. attributed to the melt/decomposition of mono-maleate Form I. The TGA of mono-maleate Form I is shown in FIG. 64. The minimal weight loss (less than 0.1 wt. %) observed in the TGA data from ambient temperature to about 100° C. indicates that mono-maleate Form I is unsolvated. The DVS of mono-maleate Form I is shown in FIG. 65. The ¹H NMR of mono-citrate Form I is shown in FIG. 66. NMR analysis shows that mono-maleate Form I contains about 1 mole equivalent of maleic acid.

### Example 22. Compound I Hemi-Fumarate Form I

Compound I hemi-fumarate Form I is a crystalline unsolvated hemi-fumarate salt of Compound I. Hemi-fumarate Form I was prepared by slurrying approximately 0.2 g of Compound I free base in 3 mL of EtOH and charging 1 mL of EtOH containing approximately 1.5 molar equivalents of fumaric acid at room temperature. The solids were isolated using a NANOSEP centrifugal filter.

The XRPD pattern for hemi-fumarate Form I is shown in FIG. 67. The DSC curve of hemi-fumarate Form I is shown in FIG. 68. The first thermal event is a baseline shift from about 83° C. to about 91° C. attributed to a glass transition. The second thermal event with an onset temperature of about 151° C. was not investigated. The TGA of hemi-fumarate Form I is shown in FIG. 69. The minimal weight loss (about 0.8 wt. %) observed in the TGA data from ambient temperature to about 50° C. indicates that hemi-fumarate Form I is unsolvated. The DVS of hemi-fumarate Form I is shown in FIG. 70. The ¹H NMR of hemi-fumarate Form I is shown in FIG. 71. NMR analysis shows that hemi-fumarate Form I contains about 0.5 mole equivalent of fumaric acid.

### Example 23. Compound I Mono-Fumarate Form I

Compound I mono-fumarate Form I is a crystalline unsolvated mono-fumarate salt of Compound I. Mono-fumarate Form I was prepared by storing Compound I hemi-fumarate Form I in a sealed vessel, which was made as described above, for 6 months at room temperature. The solids were isolated using a NANOSEP centrifugal filter.

The XRPD pattern for mono-citrate Form I is shown in FIG. 72. The DSC curve of mono-citrate Form I is shown in FIG. 73. The TGA of mono-citrate Form I is shown in FIG. 74. The minimal weight loss (about 0.4 wt. %) observed in the TGA data from ambient temperature to about 100° C. indicates that mono-fumarate Form I is unsolvated. The DVS of mono-citrate Form I is shown in FIG. 75. The ¹H NMR of mono-citrate Form I is shown in FIG. 76. NMR analysis shows that Compound I mono-fumarate Form I contains about 1 mole equivalent of fumaric acid.

### Example 24. Compound I Hemi-L-Tartrate Form I

Compound I hemi-L-tartrate Form I is a crystalline hydrated form of a hemi-L-tartrate salt of Compound I. Hemi-L-tartrate Form I was prepared by charging 1 mL of EtOH containing 1.5 molar equivalent of L-tartaric acid to a slurry of approximately 0.1 g of Compound I free base in about 3 mL of EtOH at room temperature and stirring overnight. The solids were isolated using a NANOSEP centrifugal filter.

The XRPD pattern for hemi-L-tartrate Form I is shown in FIG. 77. The DSC curve of hemi-L-tartrate Form I is shown in FIG. 78. The endothermic event with an onset temperature of about 65° C. is attributed to the loss of water. The endothermic event with an onset temperature of about 127° C. was not investigated. The TGA of hemi-L-tartrate Form I is shown in FIG. 79. The TGA data shows a weight loss of about 4.8 wt. % from ambient temperature to about 65° C. indicating that Form I is solvated, and was later confirmed by TGA-MS analysis to be water. The DVS of hemi-L-tartrate Form I is shown in FIG. 80. The ¹H NMR of hemi-L-tartrate Form I is shown in FIG. 81. NMR analysis shows that Compound I hemi-L-tartrate Form I contains about 0.5 mole equivalent of L-tartaric acid.

### Example 25. Compound I Mono-ESA Form I

Compound I mono-ESA Form I is a crystalline hydrated mono-ethanesulfonic acid salt of Compound I. Mono-ESA Form I was prepared by charging 1 mL of EtOH containing 1.5 molar equivalent of ethylsulfonic acid to a slurry of approximately 0.1 g of Compound I free base in 3 mL of EtOH at room temperature and stirring overnight. The solids were isolated using a NANOSEP centrifugal filter.

The XRPD pattern for mono-ESA Form I is shown in FIG. 82. The DSC curve of mono-ESA Form I is shown in FIG. 83. The endothermic event with an onset temperature of about 86° C. is attributed to the loss of water. The endothermic event with an onset temperature of about 123° C. was not investigated. The TGA of mono-ESA Form I is shown in FIG. 84. The TGA data shows a weight loss of about 3.5 wt. % from ambient temperature to about 86° C. indicating that mono-ESA Form I is solvated, and was later confirmed by TGA-MS analysis to be water. The DVS of mono-ESA Form I is shown in FIG. 85. The ¹H NMR of mono-ESA Form I is shown in FIG. 86. NMR analysis shows that mono-ESA Form I contains about 1 mole equivalent of ESA.

### Example 26. Compound I Hemi-Glycolate Form I

Compound I hemi-glycolate Form I is a crystalline unsolvated hemi-glycolate salt of Compound I. Hemi-glycolate Form I was prepared by charging 1 mL of EtOH containing 1.5 molar equivalent of glycolic acid to a slurry of approximately 0.1 g of Compound I free base in 3 mL of EtOH at room temperature and stirring overnight. The solids were isolated using a NANOSEP centrifugal filter.

The XRPD pattern for hemi-glycolate Form I is shown in FIG. 87. The DSC curve of hemi-glycolate Form I is shown in FIG. 88. The DSC data shows 1 endothermic event with an onset temperature of about 107° C. This endothermic event is most likely the melting of hemi-glycolate Form I. The TGA of hemi-glycolate Form I is shown in FIG. 89. The minimal weight loss (about 0.4 wt. %) observed in the TGA data from ambient temperature to about 100° C. indicates that hemi-glycolate Form I is unsolvated. The DVS of hemi-glycolate Form I is shown in FIG. 90. The ¹H NMR of hemi-glycolate Form I is shown in FIG. 91. NMR analysis shows that hemi-glycolate Form I contains about 0.5 mole equivalent of glycolic acid.

### Example 27. Compound I Sulfate Form I

Compound I sulfate Form I is a crystalline unsolvated sulfate salt of Compound I. Sulfate Form I was prepared by slurrying approximately 0.2 g of Compound I free base in 3 mL of EtOH and charging 1 mL of EtOH containing 1.5 molar equivalents of sulfuric acid at room temperature and stirring overnight. The solids were isolated using a NANOSEP centrifugal filter.

The XRPD pattern for sulfate Form I is shown in FIG. 92. The DSC curve of sulfate Form I is shown in FIG. 93. The endothermic event with an onset temperature of about 44° C. is attributed to the desolvation of sulfate Form I. The endothermic events with onset temperatures of about 126° C. and about 164° C. were not investigated. The TGA of sulfate Form I is shown in FIG. 94. The weight loss (about 2.1 wt. %) observed in the TGA data from ambient temperature to about 100° C. indicates that sulfate Form I is possibly solvated with water or ethanol.

### Example 28. Compound I Phosphate Form I

Compound I phosphate Form I is a crystalline unsolvated form of a phosphate salt of Compound I. Phosphate Form I was prepared by slurrying approximately 0.2 g of Compound I free base in 3 mL of EtOH and charging 1 mL of EtOH containing 1.5 molar equivalents of phosphoric acid at room temperature and stirring overnight. The solids were isolated using a NANOSEP centrifugal filter.

The XRPD pattern for phosphate Form I is shown in FIG. 95. The DSC curve of phosphate Form I is shown in FIG. 96. The endothermic event with an onset temperature of about 46° C. is attributed to the desolvation of phosphate Form I. The endothermic events with onset temperatures of about 146° C. and about 179° C. were not investigated. The TGA of phosphate Form I is shown in FIG. 97. The weight loss (about 4.2 wt %) observed in the TGA data from ambient temperature to about 120° C. indicates that phosphate Form I is solvated with either water or ethanol. A sample of phosphate Form I was heated in the TGA to about 100° C. and was subsequently analyzed by XRPD. The results showed that upon desolvation, phosphate Form I loses its crystallinity.

### Example 29. Compound I Hemi-HCl Form I

Compound I hemi-HCl Form I is a crystalline, hydrated hemi-hydrochloride salt of Compound I. Hemi-HCl Form I was prepared by charging 1 mL of EtOH and 0.5 molar equivalent of concentrated hydrochloric acid to approximately 0.1 g of Compound I free base at room temperature and stirring overnight. The solids were isolated using a NANOSEP centrifugal filter.

The XRPD pattern for hemi-HCl Form I is shown in FIG. 98. The DSC curve of hemi-HCl Form I is shown in FIG. 99. The endothermic event with an onset temperature of about 80° C. is attributed to desolvation. The TGA of hemi-HCl Form I is shown in FIG. 100. The weight loss (about 5.5 wt %) observed in the TGA data from ambient temperature to about 130° C. indicates that hemi-HCl Form I is solvated, and was later confirmed by TGA-MS analysis to be water. The DVS of hemi-HCl Form I is shown in FIG. 101.

### Example 30. Compound I Co-Crystal Form I

Compound I co-crystal Form I is a crystalline 2-MeTHF:water-solvated form of a co-crystal of Compound I and 4-(((S)-1-cyanoethyl)amino)-6-(3-cyanopyrrolo[1,2-b]pyridazin-7-yl)-N—((R)-2-fluoro-3-hydroxy-3-methylbutyl)nicotinamide (“Compound A”). The alaninamide stereocenter in 6-chloro-4-(((R)-1-cyanoethyl)amino)-N—((R)-2-fluoro-3-hydroxy-3-methylbutyl)nicotinamide was epimerized by heating to 70° C. in IPA. The resulting mixture of 6-chloro-4-(((R)-1-cyanoethyl)amino)-N—((R)-2-fluoro-3-hydroxy-3-methylbutyl)nicotinamide and its diastereomer 6-chloro-4-(((R)-1-cyanoethyl)amino)-N—((S)-2-fluoro-3-hydroxy-3-methylbutyl)nicotinamide was isolated from IPA and heptane at 0° C. The mixture was treated with 7-(4,4,5,5-tetramethyl-1,3,2-dioxaborolan-2-yl)pyrrolo[1,2-b]pyridazine-3-carbonitrile, palladium acetate, X-Phos and aqueous K₃PO₄ in 2-MeTHF at 80° C. to afford a mixture of Compound I and Compound A. The end of reaction mixture was treated with trithiocyanuric acid trisodium salt hydrate, activated carbon and diatomaceous earth at 40° C., then filtered over diatomaceous earth to afford a 2-MeTHF solution of Compound I and Compound A. After washing the Compound I/Compound A 2-MeTHF solution with water, concentration via distillation led to crystallization of a 1:1 crystalline cocrystal of Compound I and Compound A.

The XRPD pattern for Compound I co-crystal Form I is shown in FIG. 102. The DSC curve of Compound I co-crystal Form I is shown in FIG. 103. The DSC data shows 2 endothermic events with onset temperatures of about 43° C. and about 239° C. that are attributed to the desolvation of water and 2-MeTHF, respectively. The TGA of Compound I co-crystal Form I is shown in FIG. 104. The weight loss (about 1.6 wt %) observed in the TGA data from ambient temperature to about 65° C. and the weight loss (about 6.6 wt %) from about 65° C. to about 200° C. indicates that Compound I co-crystal Form I is solvated, and was later confirmed by TGA-MS analysis to be water and 2-MeTHF, respectively.

1. A crystalline form of Compound I: 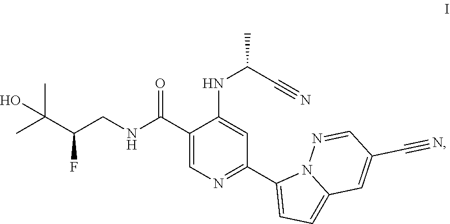 [SMILES: C[C@H](C#N)Nc1cc(-c2ccc3cc(C#N)cnn23)ncc1C(=O)NC[C@@H](F)C(C)(C)O]
(Compound I Form I), wherein Compound I Form I is characterized by an X-ray powder diffractogram comprising peaks (±0.2°) at 7.5, 12.3, and 7.2°2θ as determined on a diffractometer using Cu-Kα radiation.

2. The crystalline form of claim 1, further characterized by:
i) one or more peaks at 19.1°2θ±0.2°, 22.7±0.2°, or 15.1±0.2°;
ii) a diffractogram substantially as shown in FIG. 1;
iii) a differential scanning calorimetry (DSC) curve comprising an endotherm at about 155.3° C. (onset temperature) and an endotherm at about 174.7° C. (onset temperature);
iv) a differential scanning calorimetry (DSC) curve substantially as shown in FIG. 2;
v) thermogravimetric analysis (TGA) showing a weight loss of about 1.0 wt % up to about 150° C.; or
vi) thermogravimetric analysis (TGA) comprising a thermogram substantially as shown in FIG. 3.

3. A crystalline form of Compound I: 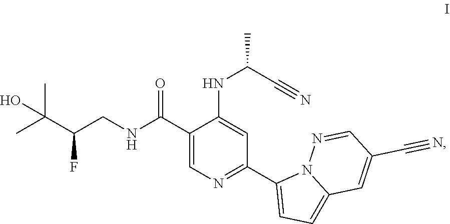 [SMILES: C[C@H](C#N)Nc1cc(-c2ccc3cc(C#N)cnn23)ncc1C(=O)NC[C@@H](F)C(C)(C)O]
(Compound I Form II), wherein Compound I Form II is characterized by an X-ray powder diffractogram comprising peaks (±0.2°) at 7.5, 14.6, and 17.8°2θ as determined on a diffractometer using Cu-Kα radiation.

4. The crystalline form of claim 3, further characterized by:
i) one or more peaks at 22.8°2θ±0.2°, 26.7±0.2°, or 22.0±0.2°;
ii) a diffractogram substantially as shown in FIG. 4;
iii) a differential scanning calorimetry (DSC) curve comprising an endotherm at about 190.7° C. (onset temperature);
iv) a differential scanning calorimetry (DSC) curve substantially as shown in FIG. 5;
v) thermogravimetric analysis (TGA) showing a weight loss of about 1.0 wt % up to about 200° C.; or
vi) thermogravimetric analysis (TGA) comprising a thermogram substantially as shown in FIG. 6.

5. A crystalline form of Compound I: 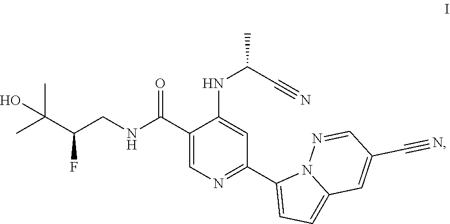 [SMILES: C[C@H](C#N)Nc1cc(-c2ccc3cc(C#N)cnn23)ncc1C(=O)NC[C@@H](F)C(C)(C)O]
(Compound I Form IV), wherein Compound I Form IV is characterized by an X-ray powder diffractogram comprising peaks (±0.2°) at 10.1, 10.7, and 17.9°2θ as determined on a diffractometer using Cu-Kα radiation.

6. The crystalline form of claim 5, further characterized by:
i) one or more peaks at 17.3°2θ±0.2°, 11.7±0.2°, or 21.7±0.2°;
ii) a diffractogram substantially as shown in FIG. 8;
iii) a differential scanning calorimetry (DSC) curve comprising an endotherm at about 192.0° C. (onset temperature);
iv) a differential scanning calorimetry (DSC) curve substantially as shown in FIG. 9;
v) thermogravimetric analysis (TGA) showing a weight loss of about 0.8 wt % up to about 200° C.;
vi) thermogravimetric analysis (TGA) comprising a thermogram substantially as shown in FIG. 10; or
vii) a dynamic vapor sorption (DVS) curve substantially as shown in FIG. 11.

7. A crystalline form of Compound I: 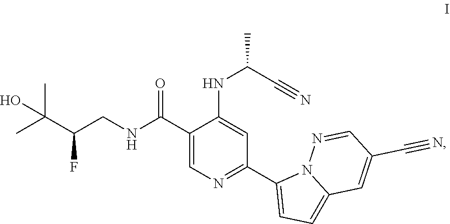 [SMILES: C[C@H](C#N)Nc1cc(-c2ccc3cc(C#N)cnn23)ncc1C(=O)NC[C@@H](F)C(C)(C)O]
(Compound I Form V), wherein Compound I Form V is a monohydrate and characterized by an X-ray powder diffractogram comprising peaks (±0.2°) at 11.8, 25.9, and 20.7°2θ as determined on a diffractometer using Cu-Kα radiation.

8. The crystalline form of claim 7, further characterized by:
i) one or more peaks at 16.8°2θ±0.2°, 23.1±0.2°, or 18.5±0.2°;
ii) a diffractogram substantially as shown in FIG. 12;
iii) a differential scanning calorimetry (DSC) curve comprising an endotherm at about 70.5° C. (onset temperature), an endotherm at about 151.6° C. (onset temperature), and an endotherm at about 189.1° C. (onset temperature);
iv) a differential scanning calorimetry (DSC) curve substantially as shown in FIG. 13;
v) thermogravimetric analysis (TGA) showing a weight loss of about 3.6 wt % up to about 100° C.;
vi) thermogravimetric analysis (TGA) comprising a thermogram substantially as shown in FIG. 14; or
vii) a dynamic vapor sorption (DVS) curve substantially as shown in FIG. 15.

9. The crystalline form of claim 7, comprising about 1 mole equivalent of water.

10. A crystalline form of Compound I: 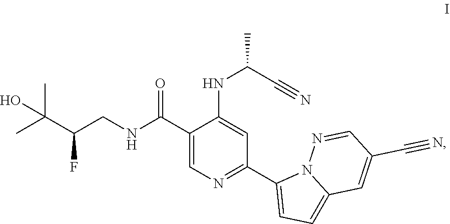 [SMILES: C[C@H](C#N)Nc1cc(-c2ccc3cc(C#N)cnn23)ncc1C(=O)NC[C@@H](F)C(C)(C)O]
(Compound I Form VI), wherein Compound I Form VI is a monohydrate and characterized by an X-ray powder diffractogram comprising peaks (±0.2°) at 4.9, 5.6, and 7.4°2θ as determined on a diffractometer using Cu-Kα radiation.

11. The crystalline form of claim 10, further characterized by:
i) one or more peaks at 8.4°2θ±0.2°, 12.3±0.2°, or 27.2±0.2°;
ii) a diffractogram substantially as shown in FIG. 16;
iii) a differential scanning calorimetry (DSC) curve comprising an endotherm at about 47.0° C. (onset temperature), an endotherm at about 111.1° C. (onset temperature), and an endotherm at about 122.9° C. (onset temperature);
iii) a differential scanning calorimetry (DSC) curve substantially as shown in FIG. 17;
v) thermogravimetric analysis (TGA) showing a weight loss of about 5.2 wt % up to about 140° C.;
vi) thermogravimetric analysis (TGA) comprising a thermogram substantially as shown in FIG. 18; or
vii) a dynamic vapor sorption (DVS) curve substantially as shown in FIG. 19.

12. The crystalline form of claim 10, comprising about 1.25 mole equivalents of water.

13. A crystalline form of a mono-citrate salt of Compound I: 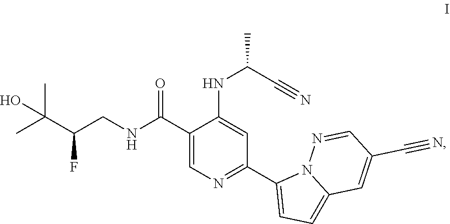 [SMILES: C[C@H](C#N)Nc1cc(-c2ccc3cc(C#N)cnn23)ncc1C(=O)NC[C@@H](F)C(C)(C)O]
(Compound I mono-citrate Form I), wherein Compound I mono-citrate Form I is characterized by an X-ray powder diffractogram comprising peaks (±0.2°) at 5.7, 7.0, and 22.7°2θ as determined on a diffractometer using Cu-Kα radiation.

14. The crystalline form of claim 13, further characterized by:
i) one or more peaks at 19.6°2θ±0.2°, 15.6±0.2°, or 8.1±0.2°;
ii) a diffractogram substantially as shown in FIG. 39;
iii) a differential scanning calorimetry (DSC) curve comprising an endotherm at about 145.2° C. (onset temperature) and an endotherm at about 186.6° C. (onset temperature);
iv) a differential scanning calorimetry (DSC) curve substantially as shown in FIG. 40;
v) thermogravimetric analysis (TGA) showing a weight loss of about 0.6 wt % up to about 150° C.;
vi) thermogravimetric analysis (TGA) comprising a thermogram substantially as shown in FIG. 41;
vii) a dynamic vapor sorption (DVS) curve substantially as shown in FIG. 42.

15. The crystalline form of claim 13, comprising about 1 mole equivalent of citric acid.

16. (canceled)

17. A pharmaceutical composition comprising a crystalline form of claim 1 and a pharmaceutically acceptable carrier.

18.-21. (canceled)

22. A pharmaceutical composition comprising a crystalline form of claim 3 and a pharmaceutically acceptable carrier.

23. A pharmaceutical composition comprising a crystalline form of claim 5 and a pharmaceutically acceptable carrier.

24. A pharmaceutical composition comprising a crystalline form of claim 7 and a pharmaceutically acceptable carrier.

25. A pharmaceutical composition comprising a crystalline form of claim 10 and a pharmaceutically acceptable carrier.

26. A pharmaceutical composition comprising a crystalline form of claim 13 and a pharmaceutically acceptable carrier.
